(19) United States
(12) Patent Application Publication
(10) Pub. No.: US 2015/0351655 A1
(43) Pub. Date: Dec. 10, 2015

(54) ADAPTIVE BRAIN TRAINING COMPUTER SYSTEM AND METHOD

(71) Applicant: INTERAXON INC., TORONTO (CA)

(72) Inventor: TREVOR COLEMAN, TORONTO (CA)

(21) Appl. No.: 14/368,333

(22) PCT Filed: Jan. 6, 2014
(86) PCT No.: PCT/CA2014/000004

§ 371 Date: Jun. 24, 2014

Related U.S. Application Data
(60) Provisional application No. 61/750,177, filed on Jan. 8, 2013.

(51) Int. Cl. A61B 5/0482; G09B 19/00; A61B 5/04; A61B 5/16; A61B 5/00; G06F 17/14; A61B 5/0205

(52) U.S. Cl. CPC A61B 5/0482; G06F 17/142; G09B 19/00; A61B 5/0205; A61B 5/165; A61B 5/742; A61B 5/04012; A61B 5/021

(57) ABSTRACT

A computer system for guiding one or more users through a brain state guidance exercise or routine, such as a meditation exercise, is provided. The computer system includes at least one computing device which may be a smart phone. A computer program which may be a mobile application runs one or more brain state guidance routines that guide at least one user through at least one brain state guidance exercise. The computing device is connected to at least one bio-signal sensor that provides biofeedback information to the computing device, and where the computer program when executed further measures performance of the at least one user relative to one or more brain state guidance related objectives by analyzing the biofeedback information based on stability of state of mind for the user. The computer program may recognize, score and reward states of meditation.

### CROSS REFERENCE TO RELATED APPLICATION(S)

This application claims all benefit, including priority, of U.S. Provisional Patent Application Ser. No. 61/750,177, filed Jan. 8, 2013, entitled ADAPTIVE MEDITATION COMPUTER SYSTEM, COMPUTER APPLICATION AND METHOD, the entire contents of which is incorporated herein by this reference.

### FIELD OF THE INVENTION

The present invention relates to bio-signal collection methods, and systems that utilize bio-signal data. This invention relates more particularly to brain signal collection and processing.

### BACKGROUND OF THE INVENTION

Various systems, devices, and computer programs for promoting a meditative state or for guiding users through meditative exercises are known (“meditation technologies”). Some prior art meditation technologies use bio-signal monitoring. Some computer programs exist that include meditation exercises. But these do not incorporate brain activity features that provide an objective measure of performance relative to meditation goals.

Bio-signals are signals that are generated by biological beings that can be measured and monitored. Electroencephalographs, galvanometers, and electrocardiographs are examples of devices that are used to measure and monitor bio-signals generated by humans.

A human brain generates bio-signals such as electrical patterns, which may be measured or monitored using an electroencephalogram (EEG). These electrical patterns, or brainwaves, are measurable by devices such as and EEG. Typically, an EEG will measure brainwaves in an analog form. Then, these brainwaves may be analyzed either in their original analog form or in a digital form after an analog to digital conversion.

Measuring and analyzing bio-signals such as brainwave patterns can have a variety of practical applications.

There is a need to improve the effectiveness of meditation technologies.

### SUMMARY OF THE INVENTION

In accordance with an aspect of the present invention there is provided a system comprising: at least one computing device comprising at least one processor and at least one non-transitory computer readable medium storing computer processing instructions; at least one bio-signal sensor in communication with the at least one computing device; wherein, upon execution of the computer processing instructions by the at least one processor, the at least one computing device is configured to: execute at least one brain state guidance routine comprising at least one brain state guidance objective; present at least one brain state guidance indication at the at least one computing device for presentation to at least one user, in accordance with the executed at least one brain state guidance routine; receive bio-signal data of the at least one user from the at least one bio-signal sensor, at least one of the at least one bio-signal sensor comprising at least one brainwave sensor, and the received bio-signal data comprising at least brainwave data of the at least one user; measure performance of the at least one user relative to at least one brain state guidance objective corresponding to the at least one brain state guidance routine at least partly by analyzing the received bio-signal data; and update the presented at least one brain state guidance indication based at least partly on the measured performance.

A method performed by at least one computing device in communication with at least one bio-signal sensor, the at least one computing device comprising at least one processor and at least one non-transitory computer readable medium storing computer processing instructions, that when executed by the at least one computer processor, cause the at least one computing device to perform the method, the method comprising: executing at least one brain state guidance routine comprising at least one brain state guidance objective; presenting at least one brain state guidance indication at the at least one computing device for presentation to at least one user, in accordance with the executed at least one brain state guidance routine; receiving bio-signal data of the at least one user from the at least one bio-signal sensor, at least one of the at least one bio-signal sensor comprising at least one brainwave sensor, and the received bio-signal data comprising at least brainwave data of the at least one user; measuring performance of the at least one user relative to at least one brain state guidance objective corresponding to the at least one brain state guidance routine at least partly by analyzing the received bio-signal data; and updating the presented at least one brain state guidance indication based at least partly on the measured performance.

A computer system or method may be provided for measuring biofeedback information of at least one user against at least one goal of a brain state exercise while the at least one user is guided through the brain state exercise. In accordance with an aspect of the present invention, the computer system may comprise: (A) one or more computer devices, (B) a computer program that when executed runs one or more meditation routines that guide at least one user through at least one meditation exercise, and (C) a bio-signal processing system that provides biofeedback information to the computer, and wherein the computer program when executed further measures performance of the at least one user relative to one or more meditation related objectives by analyzing the biofeedback information.

The one or more computer devices may include a smart phone and the computer program may be a mobile application operating on the smart phone.

The computer system may be configured to determine a user's state of meditation based on stability of state of mind. The computer system may recognize, score and reward states of meditation.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

### BRIEF DESCRIPTION OF THE DRAWINGS

Embodiments will now be described, by way of example only, with reference to the attached figures, wherein:

FIG. 1 illustrates a flow for brainstate change notification in accordance with an aspect of the present invention;

FIG. 2 illustrates an example of applying brainstate change notification in accordance with an aspect of the present invention;

FIG. 3 illustrates an example flow of adaptive brainstate change notification (ABCN) which may be implemented by an embodiment of the present invention;

FIG. 4 illustrates an example flow of an implementation of notification rules for playing feedback sounds in accordance with an aspect of the present invention;

FIG. 5 illustrates an exemplary system architecture of an embodiment of the present invention;

FIG. 6 illustrates an example of the filtering employed by an embodiment of the system of the present invention;

FIG. 7 illustrates a filter structure employed by an embodiment of the present invention;

FIG. 8 illustrates a high-level user flow of an embodiment of the present invention;

FIG. 9 illustrates steps performed to perform a signal quality check and calibration in accordance with an aspect of the present invention;

FIGS. 10A-10B illustrates calibration interface screens of an embodiment of the present invention;

FIG. 11 illustrates an example flow of a ABCN session with a brain state guidance exercise in accordance with an aspect of the present invention;

FIG. 12 illustrates various states of a status indicator of an embodiment of the present invention;

FIG. 13 illustrates an example flow of providing headband status in an embodiment of the present invention;

FIGS. 14A-14D illustrate various states of the headband status screen of an embodiment of the present invention;

FIGS. 15A-15E illustrate various states of the signal quality check screen before calibration of an embodiment of the present invention;

FIG. 16 illustrates an example of a signal quality alert during calibration or during an ABCN session of an embodiment of the present invention;

FIG. 17 illustrates an exemplary metaphor ontology of an embodiment of the present invention;

FIG. 18 illustrates an exemplary screen view of an ambient visual experience of an embodiment of the present invention;

FIG. 19 illustrates an exemplary session options screen of an embodiment of the present invention;

FIGS. 20A-20M illustrates screens that may be used to solicit and receive a user's self-report of an embodiment of the present invention;

FIG. 21-49 illustrate different possible embodiments of the computer program of the present invention by illustrating possible screenshots for the computer program, as well as possible workflow represented by transitions between the screenshots depicted;

FIG. 50 illustrates a user flow of feedback given to a user about their ABCN session(s) in an embodiment of the present invention;

FIG. 51 illustrates a data visualization user flow of screens that can be selected to view the user's results and progress of an embodiment of the present invention;

FIGS. 52A-52K illustrate examples of gamification screens indicating rewards earned or other gamified feedback;

FIG. 53 illustrates a flow for more challenging exercises to be unlocked in an embodiment of the present invention;

FIGS. 54-59 illustrate various feedback screens that may be generated by the system of the present invention to help motivate the user to train;

FIG. 60 illustrates a flow for unsupported brainstate training;

FIG. 61 illustrates a flow for supported brainstate training;

FIG. 62 illustrates a focused attention method of training without ABCN;

FIG. 63 illustrates a focused attention method of training with ABCN;

FIG. 64 illustrates overviews of the focused attention method with and without ABCN;

FIG. 65 illustrates an open monitoring method of training without ABCN;

FIG. 66 illustrates an open monitoring method of training with ABCN;

FIG. 67 illustrates an example flow for emotional appraisal;

FIG. 68 illustrates a mindfulness-based method of training without ABCN;

FIG. 69 illustrates a mindfulness-based method of training with ABCN;

FIG. 70 illustrates a compassion-based method of training without ABCN;

FIG. 71 illustrates a compassion-based method of training with ABCN;

FIGS. 72A-72F illustrate exemplary “sharing” interfaces of an embodiment of the system of the present invention;

FIGS. 73A-73C illustrate various modes of brain state guidance exercise which may be available in an embodiment of the present invention; and

FIG. 74 illustrates a generic computer used to implement aspects of the present invention.

In the drawings, embodiments of the invention are illustrated by way of example. It is to be expressly understood that the description and drawings are only for the purpose of illustration and as an aid to understanding, and are not intended as a definition of the limits of the invention.

### DETAILED DESCRIPTION

In accordance with an aspect of the present invention, there is provided a system and method for brain state guidance comprising presenting a video or audio user interface (UI) element or scene to a user which is associated by the system to encourage satisfying a brain state guidance objective for the exercise, such as achieving and/or maintaining a particular target brain state in the user. The UI element may be varied by the system based on brainwave data received from sensors worn by the user, such that the UI element is indicative of the user either achieving or not achieving the associated target brain state, thereby providing realtime feedback on changes in the user's brain state. This concept may be called Adaptive Brainstate Change Notification (or “ABCN”).

The target brain state may be chosen by the user for training by sustaining that brain state for a duration of time. The system and method of the present invention may support the user's goal state through feedback unique to the user's particular brainwave characteristics, as determined and stored in a user profile. By employing the methodology of the system of the present invention, the user may be made to be consciously aware of the goal the user is trying to achieve. Feedback may be provided by the system to help the user detect changes in brain state. Usually the change is of a discrete event. The user cultivates a meta-awareness of the state they are in. The feedback that this system provides to the user based on a classification of the brain state. The ABCN of the present invention may be self-administered by the user as explained herein.

Goal states may include: mindfulness, focused attention, open presence (open monitoring), positive emotions, and visualization, among others. Each of these goal states may be referred to as a meditative brain state, but the present invention is not limited only to meditative brain states. The present invention may guide the user to achieve or maintain other brain states as well.

The UI element or scene presented to the user may be representative of a desired brain state. For example, the desired brain state may be a calm or presence of mind state. A landscape commonly associated with calm or relaxation such as a view of a beach toward the sea may be selected. The scene may also be selected such that sound or visual elements may disturb the calm or relaxation when the user's brain state is not in the desired brain state, such as for example wind, noisy people entering the scenes, marine vehicles and the such when the user brain state is not relaxed. The user interface may provide a focal point to the user, and also permit the variation of the scene based on brain state variation during a brain state exercise. In other words, the scene may be both a focal point and, by varying visual or auditory elements of the scene, a real-time feedback mechanism to the user, for example, to provide positive or negative reinforcement based on the user's performance relative to brain training objectives.

In accordance with an implementation of the present invention, the system may determine the stability of the user's brain waves against a brain state stability threshold for the target brain state. This may be accomplished first by calibrating for the user's brain waves, by analyzing small fixed time segments of the brain wave signal, which may be called microstates, and by analyzing longer terms of the brain wave signal, which may be called macrostates, and respective statistical distributions may be determined. During a brain state guidance exercise, the system may compare the user's current brain wave data against these statistical distributions to determine a busy-mind score, such as a score on a continuum from quiet-mind 0 to busy-mind 1, which may be an estimate of macrostate. The busy-mind score may be recalculated at an interval, such as every 1/10 of a second. The continuum may be quantized into a number of segments and the system may vary the UI element or scene for the brain state guidance exercise based on the quantization segment, thereby providing a real-time brain state guidance indication to the user. As the brain state guidance exercise continues, the system may repeatedly update the brain state guidance indication in this way.

Accordingly, the user may have selected a target brain state and is therefore consciously aware of the goal that the user is trying to achieve. Through the updated brain state guidance indications, the user may also achieve a meta-awareness of the state that the user is in. The feedback that the system provides to the user can be modified by notification rules, and the system's estimates of the user's brain state may be actively adapted. This differs with conventional methods that use fixed thresholds, and are not adaptive, instead using simple rules to provide feedback.

In one aspect of the present invention, a system is provided comprising: at least one computing device; at least one bio-signal sensor in communication with the at least one computing device; the at least one computing device configured to: execute at least one brain state guidance routine comprising at least one brain state guidance objective; present at least one brain state guidance indication at the at least one computing device for presentation to at least one user, in accordance with the executed at least one brain state guidance routine; receive bio-signal data of the at least one user from the at least one bio-signal sensor, at least one of the at least one bio-signal sensor comprising a brainwave sensor, and the received bio-signal data comprising at least brainwave data of the at least one user; measure performance of the at least one user relative to at least one brain state guidance objective corresponding to the at least one brain state guidance routine at least partly by analyzing the received bio-signal data; and update the presented at least one brain state guidance indication based at least partly on the measured performance.

The at least one computing device may include a mobile phone, tablet, personal computer, or any other type of computing device. The bio-signal sensor may be part of a user-wearable headset configured with the at least one sensor to sense brainwave signals from the user. The bio-signal sensor may process and transmit brainwave data to the computing device(s). Bio-signal processing may occur at either or both of the bio-signal sensor(s) and the computing device(s).

Optionally, the at least one brain state guidance objective comprises at least one brain state stability threshold, and the measuring performance comprises determining a stability of at least the brainwave data in comparison to the at least one brain state stability threshold.

Brain-state information may be used in this invention to derive information regarding an emotional or meditative state or mood of the user. While the present disclosure discusses brain-state information in particular to derive this information, other data from the user such as from other types of sensors monitoring the user, or from other sources such as other data stored on or available to the user's computing device (e.g. the user's calendar, social media timeline, or other data sources), may be used together with the brain-state information.

The brain state guidance exercise may comprises a meditation exercise, and the updating may comprise presenting an indication of the stability of the brainwave data of the at least one user. The at least one computing device may be configured to calibrate the at least one brain state stability threshold at least partly by: analyzing fixed time segments of the brainwave data in real-time; calculating an alpha power value for each time segment; calculating a statistical distribution of the alpha power values over a predetermined calibration time segment, the predetermined calibration time segment comprising a longer duration than each of the fixed time segments; and determining a statistical distribution of alpha variability based at least partly on instantaneous alpha variability of the statistical distribution of the alpha power values. The received bio-signal data may be time-coded, and the stability determining may comprise: determining alpha power and alpha variability of the brainwave data for respective time codes; comparing the determined alpha power to the statistical distribution of the alpha power values; comparing the determined alpha variability to the statistical distribution of the alpha variability; and based at least on the alpha power comparison and the alpha variability comparison, estimating a busy-mind score on a continuum from quiet-mind to busy-mind, wherein the at least one brain state guidance indication updating is based at least partly on the estimated busy-mind score.

The continuum may be quantized into a predetermined number of quantization segments, the at least one brain state guidance indication may comprise a respective brain state guidance indication state corresponding to each quantization segment, and the at least one brain state guidance indication updating may be based at least partly on the brain state guidance indication state corresponding to a determined quantization segment corresponding to the estimated busy-mind score.

The at least one brain state guidance indication may comprise a representation of blowing wind, and the at least one brain state guidance indication updating may comprise updating the intensity of the representation of the blowing wind based at least partly on the estimated busy-mind score. The representation of the blowing wind may be increased based at least partly on the busy-mind score estimated to be in a busy-mind state on the continuum.

The at least one brain state guidance indication updating may comprise presenting a representation of a bird song based at least partly on the estimated busy-mind score remaining in a quiet-mind state on the continuum for a predetermined time period.

The stability determining may be periodically re-determined at a time interval. The time interval may be 1/10 of a second.

The at least one computing device may be configured to monitor for a statistically significant change in the determined alpha power and alpha variability of the brainwave data, and in accordance with determining the statistically significant change has occurred, repeat the at least one brain state stability threshold calibrating.

The at least one brain state guidance objective may comprise the estimated busy-mind score indicative of a quiet-mind on the continuum.

The at least one computing device may be configured to present a representation of awarding a milestone reward to the user based at least partly on the user achieving the at least one brain state guidance objective.

The at least one brain state guidance indication may direct the user to enter a quiet-mind state.

The at least one brain state guidance indication may direct the user to enter a quiet-mind state, then enter a busy-mind state, then enter a quiet-mind state again.

The at least one computing device may comprise a display, and the at least one brain state guidance indication updating may comprise displaying a visual prompt on the display for directing the user.

The at least one computing device may comprise an audio output device, and the at least one brain state guidance indication updating may comprise playing an audio prompt on the audio output device for directing the user.

The system may be configured to generate a feedback report for at least one session of execution of the brain state guidance routine providing an indication of the measured performance.

The execution of the brain state guidance routine may be time-coded, the received bio-signal data may be time-coded, and the at least one computing device may be configured to update a bio-signal interaction profile associated with the at least one user with the time-coded bio-signal data and the measured performance.

The performance measuring may be further based on data stored in the bio-signal interaction profile of the at least one user.

The at least one brain state guidance objective may be based at least partly on data stored in the bio-signal interaction profile of the at least one user.

The at least one computing device may be configured to transmit the measured performance to at least one remote computing device over a communications network.

The updating the presented at least one brain state guidance indication may be based at least partly on guidance information received from the remote computing device.

The at least one brain state guidance routine may be controlled by a remote computing device in communication with the at least one computing device over a communications network.

The at least one computing device may be configured to notify a remote computing device upon execution of the at least one brain state guidance routine.

The bio-signal data analyzing may be based at least partly on receiving brain state metadata information from the user, the at least one computing device configured to tag the bio-signal data with the received brain state metadata information, and store the tagged bio-signal data in a bio-signal interaction profile associated with the at least one user.

The at least one brain state guidance objective may be based at least partly on the received brain state metadata information.

The at least one computing device may be configured to receive brain state guidance proficiency information indicating the proficiency of the respective user in achieving brain state guidance objectives, and to select the brain state guidance routine for execution based at least partly on the received proficiency information.

The at least one computing device may be configured to generate the brain state guidance proficiency information based at least partly on the measured performance.

In one aspect, a computer system is provided that includes: (A) one or more computer devices, (B) a computer program that when executed runs one or more meditation routines that guide at least one user through at least one meditation exercise, and (C) a bio-signal processing system that provides biofeedback information to the computer, and where the computer program when executed further measures performance of the at least one user relative to one or more meditation related objectives by analyzing the biofeedback information.

The present invention may be adapted for uses other than meditation.

In accordance with an aspect of the invention, there may be provided a mechanism for determining a user's state of meditation, as further explained below. In particular, the system of the present invention may recognize, score, and reward states of meditation of a user.

In one aspect of the invention the system provides feedback to the at least one user based on the performance of the user relative to the meditation related objectives. In one aspect, the computer includes or is linked to a display, and the system provides feedback to the user on the display.

In another aspect of the invention, the meditation exercises are designed to train the user.

In one aspect, the system includes or links to a profile manager component, where the profile manager component establishes, stores on a database, and iteratively updates a profile for each user, where the profile includes information regarding recent performance of the user related to the meditation related objectives. The system may include functionality that provides positive and negative reinforcement to the user, during or after the meditation or other brain state exercise. The positive/negative reinforcement and the user's subsequent biofeedback response thereto may allow for the system of the present invention to interpret and analyze the user's brain state and thus the user's meditative state.

Optionally, the system tracks performance of the user historically. Also, the system may include functionality to track actions and performance of the user within an incident of use of the system or system session. Particularly where the system does not have historical information, extensive historical information, or a profile for the user, the computer system may determine a user's state of meditation, which was not previously possible.

In one aspect, the computer system updates the user's profile in real time or near real time, in order to track progress of the user in a single system session.

The computer program may include one or more training routines that are designed to improve the user's performance relative to meditation related objectives.

In one implementation, (A) a user accesses the computer system for example by signing in to the computer program, (B) the computer program accesses one or more routines for acquiring information from the user regarding their proficiency in meeting meditation related objectives and/or their preferences, (C) based on the proficiency information for the user and/or their preferences, the computer program selects a meditation exercise from a library of meditation exercises that is appropriate for the user based on their proficiency information. Alternatively, the user may provide to the computer program demographic information or this may be acquired from a linked Internet resource such as a social media profile, in order to enable the suggestion of an appropriate one or more meditation routines based on this information.

In one aspect, the system is adaptive to the user, based on their profile, and also based on their performance relative to meditation related objectives within a session.

Performance relative to meditation related objectives may be measured based on analysis of the biofeedback to derive brain state information. As explained below information relevant to mediation related objectives can be extracted from brain state information. In one aspect of the invention, the computer program includes an analyzer that accesses information regarding the meditation routines embodied in a current meditation exercise and relates the brain state information relevant to particular events or stages that are part of the applicable meditation routine. For example, a meditation exercise may require that a user achieve “mindfulness of breath” or “mindfulness of sound”. The brain state information and optionally other bio-feedback or non-bio-feedback information may be used to assess whether the user is achieving “mindfulness of breath” or “mindfulness of sound” objectives. These objectives may be associated with one or more thresholds to determine not only whether a user is for example attempting to meet these objectives, but possibly also the particular region achieved by a user performing a mental exercise of the present invention, where a region is defined by a range of brain state values, or values based on other bio-signal or non-bio-signal data.

In one aspect of the invention, the computer program when executed provides positive or negative reinforcement depending on the results achieved by the user.

In one aspect of the invention, the computer system is designed so that a user, within a relatively short session, and also without the need for an extensive profile built for the user, can have a meaningful meditation experience, and also within this experience can understand what is required to improve relative to meditation related objectives. In other words, the computer system is designed so that meditation training can be achieved during a discrete meditation exercise.

Various types of brain state exercises, including various meditation exercises, are possible. In one example, the computer program may guide the user through: (A) a discovery stage where one or more preferences of the user are collected from the user that are relevant to the user's meditation experience. For example, the user may be asked what colours relax them; what imagery relaxes them or meets another meditation related objective; or a description of different meditation exercises may be provided to the user, the user being given the choice of a meditation exercises that they feel best suits them. (B) The computer system may optionally access a profile for the user, and present a meditation exercise based on the results of the discovery stage, as well as the user's stated preferences. (C) The meditation exercise may include a series of visual prompts and audio instructions that guide the user through an experience that helps promote meditation. For example, the user may be prompted to concentrate on a body sensation; or move graphical user interface (“GUI”) element in a particular way. In one aspect of the invention the visual prompts and/or audio content may be adapted based on the preferences of the user and/or the profile for the user.

In one aspect, the computer program is configured to that the user is provided one or more instances of feedback.

In another aspect the profile manager component is configured to capture and store recent history relevant to the user, which is used to adapt the user experience through the computer system. For example, the history may include brain wave power levels. In another aspect, the analyzer is configured to build one or more statistical distributions of the brain state information. These statistical distributions permit the computer system to provide a desirable experience even during a short demo for example, without the need for a significant profile built over time. Statistical distribution may be created relative to various user group traits.

In one possible implementation, at least one meditation exercises is focused on “stability of mind”. There are certain brainwaves associated with a “quiet mind”, and others with an “active mind”. States of mind determined by brain waves such as a “quiet mind” or “active mind” can be difficult to sense in part because the indicative brain waves may depend from user to user and therefore accurate sensing may require a relatively extensive profile or more user feedback than is desirable. In one innovative aspect of the invention, the analyzer incorporates one or more machine learning techniques or algorithms that enable the discovery of the user's brain state in an efficient manner.

In one aspect, at least one meditation exercise is designed such that if performed successfully it will create in the user a stable state of mind. The computer system then is configured to measure how successfully the user is able to maintain the stable brain state, and based on their success a score is calculated. The idea of using brain state stability for this purpose is an important contribution of the inventors. A stable brain state within particular brain wave power level regions may indicate different meditative states such as for example “mindfulness of breath” or “mindfulness of sound” or “concentration”. The system may provide a way to capture information related to these states of meditation.

In one aspect of the invention, the computer program embodies logic that derives meditation state information.

In one particular implementation, the inventors have discovered that that the entrained process of breathing has an impact on oscillation of brain cycles. Also, through the computer program, the computer system has information regarding what the user is experiencing through the computer program, such as particular music or visual content (through the computer program's user interface), and this content (including optionally based on the profile) may be likely to produce a certain response in the user. The oscillation of brain cycles; the likely response based on content; the cycle defined by the meditation exercises, together enable the computer system to derive information regarding the probably meditation state of the user. For example, while it may be difficult to confirm whether a user is concentrating or not, it is possible to determine whether the breath cycle of the user is in conformity with the intended breath cycle based on the meditation exercises. A sufficient gap may indicated distraction, and therefore lack of concentration.

The inventors have identified that stability of state of mind is in part the absence of distraction. Therefore the system of the present invention, in one aspect, may include logic and processes for detecting the likelihood of distraction when the user is required to be, in accordance with the meditation exercise, in a state of stability. Accordingly, lack of stability means that the user is not in the desired brain state.

In one aspect of the present invention, the user may be directed to touch a screen linked to the computer device in a particular way if the user is distracted, or the user may be asked if the user is distracted and invited to come back to the desired meditation state based on the meditation exercise.

In one aspect, the computer program captures information regarding distraction and stability mind, and associated brain bio-signal data in order to enable modeling of brain state indicators relevant for the particular user to their states of meditation. This modeling enables the computer system to learn when the user is becoming distracted perhaps before they know this themselves. The commencement of distraction may trigger in the computer system more relaxing music or other prompts or content meant to help the user in following the meditation exercise. The content or other prompts being used by the computer system themselves may be the source of the distraction, in which case the computer system may adapt the content or prompts automatically based on the knowledge extracted by the analyzer by modeling meditation states using brain state indicators and relating this to computer program “events”.

In accordance with an aspect of the present invention, accurate sensing may be achieved by designing the computer system to focus not on the presence of a “quiet mind” or “active mind” but rather on brain stability. More specifically, the computer program when executed detects brain stability, and these are mapped to instructions built into the meditation exercise to provide an engaging meditation experience in the context of a finite session. In one aspect, the computer program is configured so that it is assumed that a user is attempting to comply with the instructions.

In other words, prior art solutions attempt to map brain state to activity very closely, which is challenging. Rather, the computer system of the present invention may be configured such that stability information is captured, and this is mapped to instructions or other parameters in the brain state guidance (e.g. meditation) exercise so as to measure performance of a user in connection with a particular exercise.

In one aspect of the invention, feedback provided by the computer system may include a meditation score. Further rewards or incentives may be linked to scores. A score may be based on stability for example. Scoring may be linked to gamification to motivate users and also to make the computer system more engaging. Sound and visual stimuli may be used to enhance the overall experience.

The biofeedback processing system may also be used for example to analyze the user's reaction to the feedback from the system. For example, the analysis can determine whether system feedback intended to be “positive” actually elicits a positive response.

In another aspect of the invention, the computer system may rely on data stored regarding mediations of other users, and also the computer system may be linked via a computer network to other computer systems so as to access in real time or near real time information that enables the derivation of patterns so as to identify for example current trends in users interacting with particular meditation routines and also so address external factors such as distractions in a space with multiple users. The computer system may be adapted to interpret events by relating this to the particular instance that the user is experiencing in their cycle established by the computer program.

In one possible implementation, the computer device may be any manner of mobile device such as a smart phone or a tablet computer. The mobile device may connect wirelessly to a headset that operates as a bio-signal processing system. The mobile device may itself include one or more sensors.

The mobile device may be part of a trusted network of local client devices 113 and for example an associated software as a service (SAAS) platform. The trusted network of local devices may comprise, without limitation, one or more of the following, in various combinations: a mobile phone, wearable computer, laptop, local computer, or trusted server.

Trusted local client devices may communicate via a network connection, for example, a wired or wireless internet connection. Furthermore, the devices in the trusted network may be configured to communicate with each other. Systems and methods for local networking between devices are known and non-limiting examples of such methods are wired, wireless, local peer-to-peer, and BLUETOOTH™ networking.

Examples of sensors include internal sensors, external sensors, wearable sensors, and user effectors operatively connected to the trusted network of local devices or the mobile device. Sensors for collecting bio-signal data include, for example, electroencephalogram sensors, galvanometer sensors, or electrocardiograph sensors. For example, a wearable sensor for collecting biological data, such as a commercially available consumer grade EEG headset with one or more electrodes for collecting brainwaves from the user.

According to an embodiment, systems and methods for a meditation biofeedback application are provided. The embodiment comprises a mobile meditation solution including an EEG headset bundled with an application that measures a user's brainwaves while s/he meditates and tracks their progress over time. The product may be configured to be functional out of the box and deliver clear and compelling benefits (reduced stress, improved mood, increased effectiveness in the workplace, etc.) The application may be implemented to a range of different platforms such as iOS™ and Android™, and may: help people learn to meditate; allow them to observe their progress and provides motivational support for their meditation practice; allow users to build and participate in communities of like-minded meditators, for example by enabling real time coaching from a remotely located meditation coach using the system of the present invention; using the latest research in the EEG/meditation field to give the user the most accurate measures possible to facilitate their learning.

In one implementation, based on permission by a first user, a second user may be notified when the first user is using the meditation application, and may enable the second user to access a dashboard that enables in real time or near real time to track the progress of the first user. The system may enable the second user to provide encouraging guidance or messaging to the first user for example by providing a voice link or to enable the integration of messages from the second user into an interface presented by the client application of the first user.

In one implementation, a user wears a brainwave headset that is connected to their mobile phone while they meditate. An application on the phone processes the brainwaves and gives them feedback about their brain state using neurofeedback to help them achieve deeply meditative states and to speed their learning of meditation states. On the mobile device, the user's data is recorded and will be used to generate a personal meditation history that shows their progress over time.

The user's data can be processed and analyzed on the client device, or the data can be uploaded to a cloud database for processing and analysis.

In this example, the user's data is uploaded to a cloud database where machine learning algorithms can process the data and thereby customize the brainwave processing algorithms to better fit the user's brainwaves. The adaptations are then offered back to the user through their online profile, to enhance their experience.

The application of the present invention may provide a number of features or capabilities. One feature of the present invention may include live group guided meditation, where the instructor receives real-time information about the brain states of the subjects. Subjects may also be able to receive updates from each other including real-time brain state and other relevant measurements such as breath phase.

Another feature of the present invention may include integration of pre- and post-meditation collection of subjective experience factors (such as their mood and level of alertness to support more comprehensive results to the user and provide features to be used in the server side algorithm improvement engine.) This allows the user to track qualitative aspects of their practice and see trends that can improve motivation and speed progress. Survey answers also allow the meditation experience to be tailored to suit user preferences. The present system includes a social media layer that allows users to “friend” one another based on shared attributes such as similar interests, similar profiles (including for example similar pace or challenges in achieving meditative states for example or differences in profiles that suggest that a first user may be able to assist a second user in achieving defined objectives). The social media layer may enable a variety of social interactions between users, including for example support provided by one user to one or more other users in achieving goals of applications. Support may be expresses for example by sending supportive messages, encouraging digital media objects such as badges and so on. Various implementations are possible.

Another feature of the present invention may include cloud-based extensible user profiles for personal information; settings and algorithm parameters that are continuously tuned (through the use of the application) to be improved or optimized for the specific user; hardware setups and application specific parameters; ad relevant training data. This will allow application usage to be device independent, and allow for back compatibility as the application is improved.

Other features of the present invention may include: program tools to download brainwave & associated data from cloud and convert into MATLAB™ and PYTHON™ data formats may be provided; integrated audio feedback to allow the user to know when headset signal quality is poor, or when other program functionality is impaired; a meditation timer with bell stimulus that can be time locked to EEG analysis to support stimulus entrainment ERP analysis; iPod™ player integration (or integration with other wireless devices), to synchronize music with EEG collection to support time locked stimulation and analysis relating to music (this may be related to measuring meditation performance as well as music recommendation system); incorporating an attentional blink cognitive test for meditation progress scoring; use of acoustic entrainment based meditation performance measures; including multi-stable perception analysis, using visual illusions such as the Necker cube, Schroeder staircase and Rubin's vase, to measure EEG dynamics related to cognitive re-framing; choose your own adventure style decision tree for meditation education and presentation sequence, constructive feedback and encouragement; environmentizer for voice in guided meditation; Mr. Potato head body scans; Chest and head breath movement analysis; Hands fidget analysis, hands breath counting; phase locked loop rhythmic entrainment including rocking of body, rhythmic breath, rhythmic alpha and or theta, and Paired Synchronization meditation; standing balance meditation and yogic poses before and during meditation; augmented reality meditation environment: visual world changes when in different phases of meditation.

In one implementation, brain wave data is uploaded to the cloud service, where machine learning algorithms adapt the algorithms and send that information back to the mobile device. In one aspect, machine learning algorithms are used to detect stability of mental states

Traditionally methods work by having the individual maintain meta-attention on their attention and self-monitor when they drift. The present invention provides a novel teaching method for supporting focussed attention training (mindfulness of breath). The key is to develop intention, attention, and a specific attitude towards all of this. The present invention may provide a support mechanism (wind feedback) when the user attention drifts. This is different from a conventional therapy where one is compelled to generate a certain state as opposed to being aware of the practice they are engaging in.

### Microstate Classification

Microstate classification, as implemented by the present invention, describes how small atoms of thought may be used to estimate brain state. Microstates are transient, patterned, and quasi-stable states of an electroencephalography (EEG). These brief states, or microstates, tend to last anywhere from milliseconds to seconds. These transient periods are thought to be the most basic states of human neurological tasks and are nicknames “atoms of thought”. An example of a microstate is known as an alpha burst. These are brief segments from a few hundred milliseconds to several seconds that are high amplitude EEG waves that have a frequency of approximately 10 Hz. Alpha bursts are associated with inhibitory processes in the brain that suppress sensory input from reaching higher levels of consciousness. This is very important to focussed thinking so that humans can suppress irrelevant sensory input such as distraction conversations or noise or discomfort in body sensations and focus at a task at hand. The most prominent occurrence of alpha bursts occur when a person closes their eyes and the number of alpha bursts increases dramatically compared to eyes open.

There are other microstates that have been identified. One taxonomy is the 4-class microstate topographies: Class A (Auditory): right-front higher amplitude than left-back, Class B (Visual): left-front higher amplitude than right back, Class C (self-referential): front and side-to-side stronger amplitude than rear, Class D (Attention): only front higher amplitude than rear and sides. The duration of microstates are often of interest in identifying disorders such as schizophrenia. And the amount of time spent in a specific microstate is of interest. In this classification microstates are typically 100 ms long.

Combining microstates into other categories may be referred to as macrostates. These macrostates are determined by analyzing patterns of microstates, such as specific order of sequences, rate of microstates per second, or other information. A macrostate may be as short as a few microstates or many hundreds of microstates that are related to higher level cognitive processes.

Conscious-brainstate may be defined as a set of one or more macrostates that a human is aware of or can learn to be aware of.

Accordingly, a microstate may be thought of as a an atomic brainstate of a duration of approximately 10 ms to 3000 ms in length, that are the fundamental building blocks from which features are extracted. Examples of signal fragments are evoked response potentials, alpha bursts, etc. A macrostate may by thought of as a pattern of signal fragments that indicate a change in brainstate that is meaningful for the goal the user is trying to achieve. Brainstate Change Notification refers to providing information to the user to help them become aware of their brainstate change. In addition to audio, visual or tactile notifications, additional forms of stimulus can be applied for therapeutic reasons. Change detection without labels refers to an approach to build local time statistics for a period of time (e.g. 5 secs, 10 sec to a minute) until local stability of the distribution is achieved. A statistical distribution of relevant features may be determined while the user does a prescribed brain exercise. A brainstate estimate may have two primary components: a determination of change significance, and a determination of the direction of the change. The system may attempt to determine whether the distribution has changed, optionally using statistical tests to do this, such as t-test. The system may control for false positives by how quickly the user responds plus prior domain knowledge. The system can derive feedback by frequency of changes plus direction of change.

### User Annotation

User annotation refers to ways in which the user can annotate their brainstate to improve the models that are built by the system of the present invention for estimating brainstate. User annotation may be used to label time segments of the data to supervise machine learning to develop models and rules for brainstate estimation. User responses can be made to be fun and engaging. The following describes different strategies for user annotation. The User Annotation also makes the system adaptive as it gets personalized to each user.

User annotation may be carried out by gamification. The brain training system may have a model for the user but its accuracy needs to be improved. The user enters a mode to improve accuracy of the model. Using its existing model the system calculates the probability of being in specific brainstates. When a threshold for probability has been surpassed a tone indicative of this state is played to the user. The user assesses their current state compared to the tone and enters whether the system's characterization was correct or not. An enhancement is to reward the user every time they catch this tone with points.

A video game can be thought as annotation plus stimulation. The system will classify multiple states and the user will control a game with their mind. The goal will be easier to reach when the goal state is exhibited. So the user may exhibit brainstate A, B, and C etc. Let's say that A is the goal state. When the system model estimates that A has occurred based on features, A the game responds with an action, i.e. stimulus, and responds with a signal that the user is in Brainstate A. If the system detects that the user evoked an ERN, i.e. the system is wrong. The confidence of this model is called into question. Let's say that the system has learned a model (m1 for model 1) using a sequence of microstates x, i.e. m1(EEG microstates x)=brainstate A.

For example, the user may annotate desired states or undesired states. The user may be interested in understanding and improving their creativity. They tag when they are being creative. The user may enter a state of creative flow between noon and 2. He tags that he is in this desired state. Their user profile labels the recorded EEG data during these periods as creative flow. The EEG data is analyzed for its pattern of microstates and macrostates and compared to known patterns. Machine learning is used to discover new patterns. The system will be trained to provide feedback whenever the user is in the desired. The user is being coached and notified to sustain that state.

There are different ways that the user can annotate, including: System can prompt the user; User can tag (e.g. Trigger is a button on the APP); Could be a sound to prompt user to annotate or simply the sound as a stimulus that the EEG is analyzed to determine the user's evoked response; Could be an electronic calendar that shows the user is engaged in a specific activity; The user may use a gesture to annotate; and Tagging can be linked to another bio-signal such as statistics from heart rate variability.

Gamification may also be adapted with User Annotation, including: structure rewards around annotation; reward user on the accuracy of their annotation; punish for false annotation; gamify annotation—reward annotation; user control of annotation are game controls; and A video game can be defined as user annotation plus stimulation.

### Notification Rules

Notification Rules refers to how the brain state estimates are turned into meaningful feedback to the user that helps them achieve a goal.

Change is a discrete event that the user is aware. The system may be configured to attempt to train meta awareness like brainstate change detection. The meta awareness is of moment to moment events that the user is consciously aware. Events that are too long or too short means that the user will be unable to relate to these events.

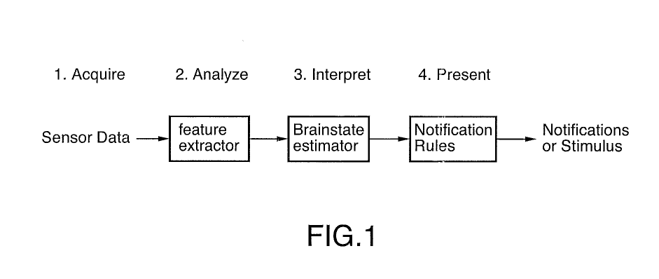

FIG. 1 shows a flow for brainstate change notification in accordance with the present invention. Step 1 is the acquisition of the signal into sensor data. Step 2 is analysis by feature extractor. The feature extractor analyzes fragments of the sensor data and extracts features of that fragment. The size of the fragment could be fixed time window or could vary based on the characteristics of the sensor data itself. Examples of signal fragments of variable length that are discovered in the brain signal are microstates and alpha bursts. Step 3 is Interpretation. The Brainstate estimator classifies features into a stream of brainstate estimates. The brainstate estimator could use unsupervised learning like deep learning or modelling the signal. However, the clusters that the unsupervised learning discovers are labelled as brainstates that the user needs feedback. Step 4 is notification to the user. The notifications sent to the user need to be relatable to their human perception. Some changes in Brainstate may not meaningful or useful for the user to receive notification, or the changes in brainstate need to be processed through rules that can send notifications that are effective in helping a user achieve their goal. The Notification Rules also examine the rate of changes in Brainstate and apply rules or filters (linear or non-linear) that have temporal relevance to the user. For instance, the brainstate estimator is detecting changes of state that are too fast for any human to perceive. The Notification Rules can use, as an example an integrator that counts the number of events that are indicative of the target state until a threshold is reached and then a notification is sent to the user. Another way of doing this is to apply a deadband after a notification that prevents any further notifications being sent to the user so they are not overwhelmed with notifications. The Notification Rules may apply discrete or continuous notification to the user.

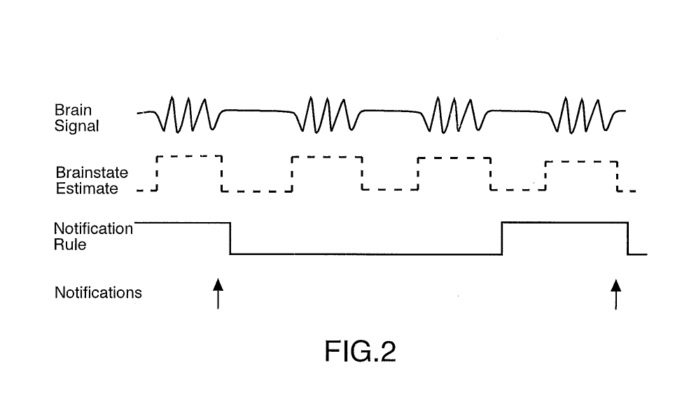

FIG. 2 shows an example of applying brainstate change notification. The first signal shows the an EEG signal of alpha bursts that are 10 Hz in frequency. An alpha burst is used by the brain to inhibit specific sensory pathways so they do not interfere with conscious awareness. The user has selected a goal that they are trying to improve their ability not to be distracted by irrelevant stimulus so they can focus on a task at hand. Notifications will be sent to the user to let them know they are successfully suppressing external stimulus. The second line (i.e. dashed line) shows that the Brainstate estimator has determined the start and stop of each alpha burst. A high value of the dashed line indicates an alpha burst in progress. The third line is a rule in the Notification Rules that prevents further notifications to be sent to the user until the end of a wait period shown by the high value of the Notification Mask. The Fourth line shows up arrows that indicate when a notification was actually sent to the user.

Notification Rules may decide what events or brainstates are interesting to the user. The rules consider the type of video game or exercise the user is engaged and the goal that user is trying to achieve. Notifications may be sent to the user so as to not be too frequent, jarring or volatile.

Notification Rules Types may include: Continuous Feedback (proportional), where feedback received is usually linear and proportional to a measure of a signal (e.g. speed of a race car is linearly proportional to alpha power) Continuous Feedback (integral), where feedback is proportional to how long user maintains a measure usually above a threshold (e.g. Speed of race increases as user maintains alpha power above a threshold); and Discrete Feedback, where Reward or penalty is a discrete event like a bell ding.

System Adaptation while in Session

The system may adapt while in a brain state guidance exercise session to the changing characteristics of the user and their environment.

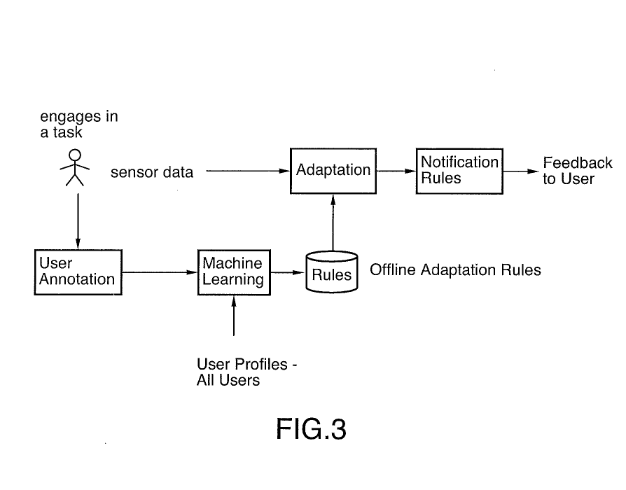

FIG. 3 shows an example flow of ABCN which may be implemented by an embodiment of the present invention. Conventional systems provide fixed thresholds that are set prior to the start of a session based on the user's previous sessions or a database of normative data. The present system may adapt a session as shown.

Covariate shift adaptation may be an effective method to adapt to sessions without the need for building a new model for the data. Covariate shift is defined as the situation where the training input points and test input points follow different distributions while the conditional distribution of output values given input points is unchanged. An example of covariate shift in EEG-based brain-computer interfaces occurs when, given different experimental sessions of the same imaginary tasks, event-related synchronization/desynchronization cortical distributions remain unchanged, but the means and variances shift in the feature distribution for each task.

Some reasons why it may be desirable to adapt a session, include where a user becomes tired, the difficulty level of the brain state guidance exercise may need to be adapted to allow for the user having less control over the user's brain state in a tired state. It may also be advantageous to adapt a session where subtle variations in the environment such as ambient temperature, noisy surroundings, or internal distracting sensations (e.g. leg hurts, hungry) are occurring.

The present invention differs from conventional systems at least in how the present system labels the data that adapts to the user that uses prior statistical knowledge of the user, and the use of stimulation and a person's reaction to it. This may result in more accurate and timely feedback. The microstate state machine of the present system may provide better estimates in noisy EEG because of prior knowledge. The system may provide for re-labelling of past states with knowledge gained in the future states. For example, the system may look at parts of the data where the mind was busy and quiet in the context of state transitions. This may inform that the microstate transitions of Quiet, Busy, Quiet, Busy, is not likely.

In accordance with a non-limiting implementation, firstly, a calibration process may be performed. A large region of the brain may be used to measure EEG signals (e.g. measured from front to back and or left to right). Optionally, brain signals from both left and right hemispheres are measured (e.g. left mastoid, under ear, to forehead and right mastoid to forehead). Small fixed time segments of the EEG signals may be analyzed in real time. The alpha power for the time segment is calculated. A longer term (e.g. 15 seconds) may be used to calculate the statistical distribution of alpha power during this term. The instantaneous alpha variability is determined as a range of values of the distribution of the alpha power. This is used to build a statistical distribution of alpha variability.

The Calibration may produce two statistical distributions: Alpha Power Distribution, Alpha Variability Distribution. The EEG signal of the same brain region measured during calibration is acquired and analyzed to determine its moment by moment Alpha Power (AP) and Alpha Variability (AV). The real-time measures of AP and AV are used to look up their value in the statistical distribution. A mathematical function uses the probability as determined from the AP distribution and AV distribution to calculate a Busymind score. The 0 to 1 score produced is on a continuum from quiet-mind 0 to busy-mind 1. The 0 to 1 is an estimate of macrostate.

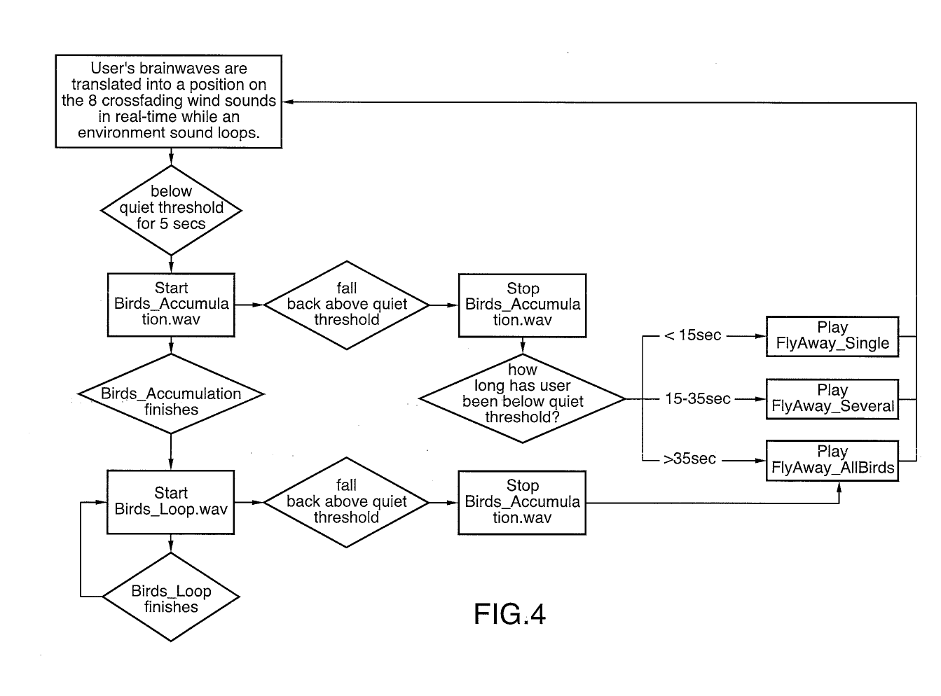

In a non-limiting implementation of the present system, the system may implement notification rules for playing feedback sounds, as shown in FIG. 4. The Busymind score varies from 0 to 1 and is updated every 1/10 second. The 0 to 1 score is on a continuum from quiet-mind 0 to busy-mind 1. The score 0 to 1 is quantized into 8 segments. Each of the 8 segments corresponds to a looping wind sound sample. The system cross-fades wind sound smoothly as user fluctuates from one segment to another. The Busymind score is also quantized into 3 equal sized segments. The highest segment is labelled active mind. The lowest segment is labelled calm mind. The midsegment is neutral. If the user stays in the calm segment for a prolonged period (e.g. 10 seconds) bonus sounds play such as bird song. Bonus sounds are carefully selected to emphasize a calm state and minimize distraction and provide a subtle reward. When the user rises back to the neutral segment after earning the bonus a sound is played to indicate a loss of stable state (e.g. birds all fly away and their song can no longer be heard). There are three different fly away sounds used depending on the number of bonus birds that are singing to stay consistent with the soundscape the user experiences. On the rare occasion the user stays at 0 for a prolonged period (5 seconds) a subtle musical rewarding sound is played.

While in session a distributions of Alpha Power (AP) and Alpha Variability (AV) are being built. When there is a statistically significant change in the AP and or AV distribution then the new distributions may be used to calculate the Busymind score, thus adapting the session to these changes.

### System Architecture

An implementation of a system architecture of the system of the present invention will now be described with reference to FIG. 5. The EEG headsets of the present invention that provide the brainwave sensors are Bluetooth-enabled devices and include a Headset Connector. The headset relies on a standard Bluetooth Socket Service. The socket service connects to a Bluetooth device using the device's Bluetooth address. It listens for Bluetooth messages transmitted from external devices. These messages are passed to the Decoder Thread. The Bluetooth pairing protocol discovers bluetooth devices and connects with them as described prior art. The BSS goes into discovery mode, it scans the local area to find Bluetooth-enabled devices nearby. The headset is set to be discoverable for the scanning procedure meaning it listens for scan requests originating from an inquiring device (in this case, the BSS). Upon receipt of a scanning request, the headset will respond by sending the inquiring BSS information about itself so that the pairing procedure can be initiated by the BSS. The BSS must have a matching Bluetooth profile required for exchanging data with a discovered device that it wants to pair with. Past pairings are remembered on the headset and the client device, including the corresponding Bluetooth device name, address, etc., in order to support auto-reconnect.

The Transmit (Tx) Command Thread sends commands to the Muse EEG Headset. Presets are commands that configures options in the Headset. The following can be turned off and on the fly: Analog Digital Conversion (ADC) settling time; Sampling rate; Bits per sample; Type of compression or no compression; Keep Alive signal, a periodic signal sent by the headset to indicate that the headset is alive and functioning which may contribute to the reliability of the system by alerting other components that communication with the headset is on—if keep alive messages are not received then the software can execute recovery procedures and alert APP that the headset is no longer connected or functioning properly; Test and debug signals; and Send accelerometer data, as the headset may include a 3-axis accelerometer, and a preset command can be sent to the headset to request accelerometer data to be transmitted/not transmitted from the headset to the computing device. The Tx command thread can also poll the Headset to determine its version, and preset settings, battery level etc.

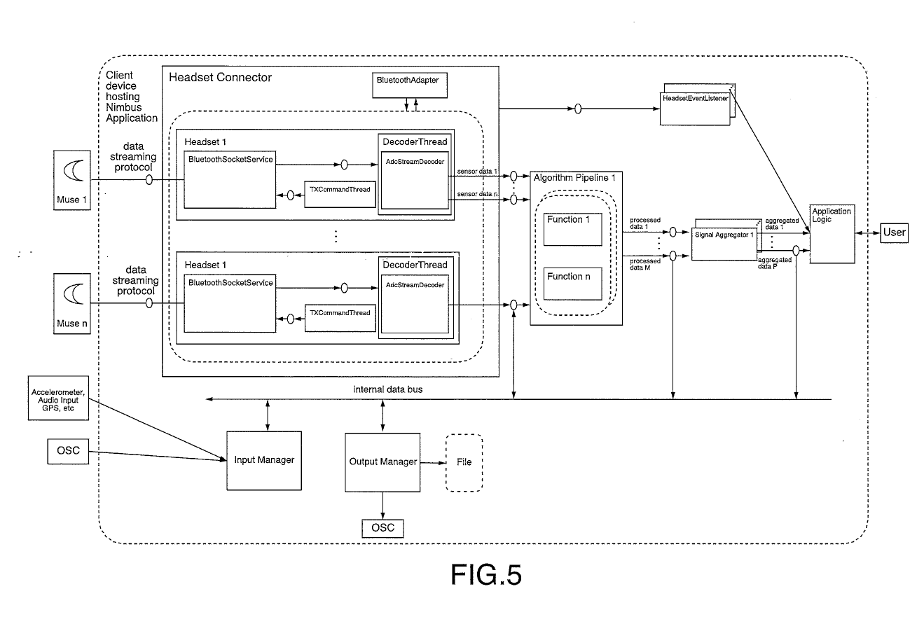

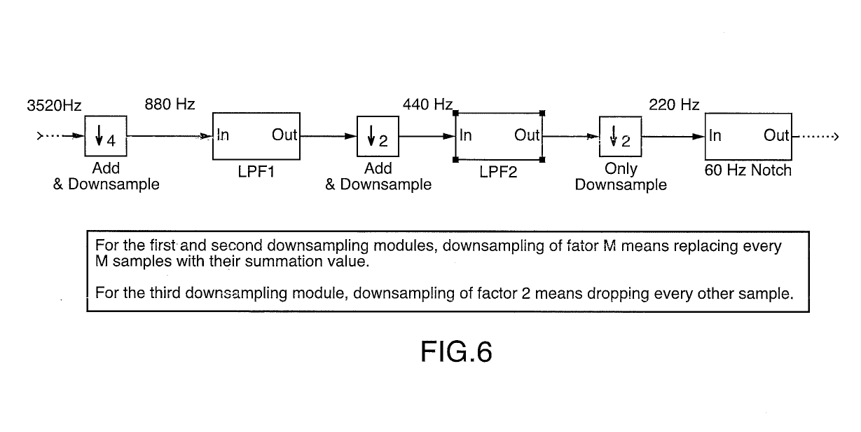

The system may employ a variety of filters, including: Different notch filters depending on geographic region (50 Hz Europe, 60 Hz NA); and Other EEG filter settings such as low pass, high pass filter settings. An example of the filtering employed by the system is shown in FIG. 6. The input signal is the 10 bit output of ADC. The sampling frequency of 3520 Hz was the largest sampling frequency that would guarantee enough processing time for the micro controller, and at the same time, after the factor of 16 of total downsampling, reduces to the 220 Hz final sampling rate of filtered data samples. The other advantage of the high sampling frequency is to minimize sample aliasing. Aliasing adds distortion to the samples if there are frequency components present in the original detected EEG signal that is greater than half the sampling rate. The 220 Hz can be transmitted on iPhone BlueTooth channel, and also is enough for the frequency range of interest of almost 0-90 Hz. All the operations are in fixed point precision. The cascade design provides reduced computational complexity at each stage, while avoiding any aliasing through enough low-pass filtering. Summation of every 4 samples results in a rough low-pass filtering that, together with the preceding analogue Low Pass Filter (LPF) in the headset, provides a safeguard against aliasing.

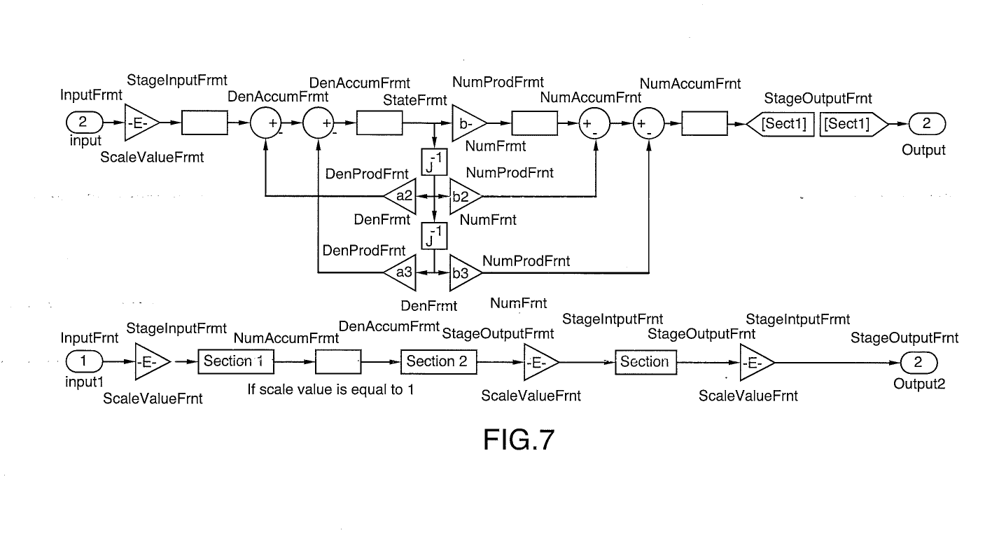

The first 2nd order LPF at 880 Hz guarantees around −40 dB attenuation of aliasing components for the frequency range of interest (0-90 Hz) during downsampling. The Fixed-Point precision design is based on the model for the adopted SOS-Direct Form II filter structure shown in FIG. 7.

Whenever two samples are added together, an extra integer bit is needed for the representation to avoid overflow. The ‘qreport’ logging is first used to test for any overflows for a test signals. After they are resolved, the fvtool can be used to compare the quantized filter response with the actual double precision filter. The operation of LPF1 and LPF2 (both sections of LPF2) are not very sensitive to slight changes in the sampling rate, but the notch filter needs to be redesigned if there are any changes in the sampling rate. The Tx command thread can also updates the firmware of the headset. Both the Headset Connector and Application logic will have the ability to update the firmware.

The system may be firmware-updateable. The firmware may be updated from a cloud server periodically, or triggered by usage of the system.

### Data Streaming Protocol

The following describes the data structure of signal data sent from the headset to the client device APP, in accordance with a non-limiting exemplary implementation of the present invention. The data is sent in a continuous data stream from the headset when CMD_TX_START has been sent, either through a command or a preset. It can be stopped by sending CMD_TX_HALT. All packets have a leading byte as header that contains a nibble for packet type and a nibble for flags. Packets are either static sized or dynamic. In the static case the length is deducted from the current settings and data type, in the dynamic case there is a length field. Also headers can be dynamic sized, the size can be deducted from the flags in the header byte.

A synchronization packet sends the absolute value of voltages detected from the headset. Subsequent packets carry the change (delta) in voltage per sensor from the previous packet. The analog voltage is digitized into a certain range and then filtered and compressed/quantized into a certain number of bits to increase its dynamic range. A synchronization packet is sent at fixed intervals every X seconds. Each packet transmitted from the headset is numbered consecutively. The Headset Connector checks the packet counter to ensure that there are consecutively numbered to determine a dropped packet. If a dropped packet is detected then the Headset Connector sends a request to the headset to send a new synchronization packet to restart the sequence with correct voltage values. In other words, synchronization resets the voltage values if a packet is dropped.

The first nibble in the first byte of the header declares the type, the second nibble is a bit array, which will be used in special circumstances. In most cases this second nibble will be zero, indicating everything is nice and in order. The additional header data is attached in the order of magnitude of the flags, highest flag comes first, lowest last.

A type nibble contains identifiers, so all 16 values can be used and will translate into a single data type (e.g. 0xf reserved; 0xe uncompressed eeg/adc sample packet; 0xc compressed eeg/adc sample packet; 0xb battery level sample packet; Oxa accelerometer sample packet; and 0x0 reserved.

A flag nibble may be ORed together, so we only have 4 flags available. If it's zero there is no more information in the header and payload starts right away (e.g. bit 4 (0x8)—samples dropped; two byte number (short) added to header; specifies numbers of samples dropped since last successful package of same type.

Payloads carry the raw signal data detected at each sensor. Payloads are determined by the type nibble, each type has its own packing and unpacking methods for the types.

A sample uncompressed EEG/ADC packet may include one payload containing a set of all sampled ADC channels, except battery (considered secondary). In regular configuration this means 4 channels. The payload is bitstuffed, so for 10 bit length samples the standard 4 channel configuration package is 5 bytes long. There are 3 different sample lengths to choose from, 10, 16 or 24.

For a compressed EEG/ADC sample packet, as we only have a reduced data rate with iOS the main consumer user case can also be reflected, that is 4 channels and 10 bit wide samples.

An accelerometer sample packet may include Bitstuffed 3 by 10 bit wide samples.

The Decoder Thread may include an ADC Stream Decoder which may parse and decompress packets (as described in Data Streaming Protocol section above) received from the Headset. The output of the ADC Stream Decoder may be a sequence of timestamped digital signal voltage values from each EEG electrode. Each decoded stream is sent to the Algorithm Pipeline.

### Algorithm Pipeline

The Algorithm pipeline was previously described in the Applicant's PCT Patent Application No. PCT/CA2013/000785, the entirety of which is incorporated herein by reference. The input to the Algorithm Pipeline are the set of N sensor data streams from the ADC Stream Decoder. One sensor stream is associated with a single sensor. For example, a single headset can have 4 electrodes. Each sample in a sensor data stream is also timestamped. An algorithm pipeline can be customized per sensor data stream.

An application developer designs an algorithm pipeline by putting together building blocks of functions that include signal processing, feature extraction, classifiers etc. The building blocks can be connected so that a data stream or information transferred within the algorithm pipeline can be split to create branches and recombined. Combining or aggregating signals can happen further down the chain. Any architecture of building blocks (splitting, branching etc.) can be assembled depending upon the algorithm that is needed.

A different pipeline can be applied to each sensor data stream. Each pipeline can be customized to different sensors based on sampling rate or characteristics of the biological signal being analyzed. Therefore separate sets of building blocks, i.e. algorithm pipelines, can be built for the same sensor types but located at different locations on the human body and or different sensor types acquiring different biological information (e.g. 4 EEG electrodes at different points on the scalp and 2 ECG electrodes acquiring heart rate data).

The signals processed by the algorithm pipeline can be sent to multiple outputs. Any number of listeners in the client device can tap into this output. The signal processor can also include estimates of the brain state like a scoring function related to a desired brain state. The output of the Algorithm pipelines are M processed data outputs. M may be greater than N, the same or less than N where N is the number of sensor data streams.

### Supporting Functions of System

The system may include a Signal Aggregator implemented on the computer device, combining processed signals coming from Algorithm Pipelines. It can aggregate across all processed signals and its output can be a mathematical function across all of the processed signal inputs: e.g. time syncing, normalization of power to another channel. As an example we can reject data in another processed data stream if an unwanted artifact is found in another processed data stream. The Signal Aggregator can output up to P aggregated data outputs that are sent to the Application Logic.

The system may include a Headset Event Listener implemented on the computer device which listens to headset and sends events to the Application logic. It may respond to non streaming events from headset such as: low battery, poor signal, and headset off.

The system may include an Input Manager which handles connections with other third party sensors like ECG, accelerometers, audio etc. It can also connect to streaming data from a LAN or internet. Open Sound Control (OSC) is a communication protocol among computers that can carry live streaming data. Other live streaming protocols such as User Datagram Protocol (UDP) may also be used.

The system may include an Output manager which takes data from various points in the application and manages where they are stored and structures their format. This data is continuous digital timestamped data. The Output Manager may stream packets to the network (e.g. internet, Cloud platform or LAN), stream packets to a peripheral or peer of the client device, and/or store streaming data to an internal file in the client device.

### Histogram Based Artifact Detection

Artifacts are unwanted signals in an EEG signal that are not generated from brain signals. Artifacts can be generated from eye movements, eye blinks, heartbeat, jaw clenches, chewing, and contraction of facial muscles. Signal power based approach for artifact detection is not able to detect subtle artifacts. The signal power for subtle artifacts such as electromyographic signals from small muscle contractions does not have large power. For artifacts in general and subtle artifacts in particular, the full Power Spectral Density (PSD) function will be more discriminating. The histogram of subtle artifact PSDs across a number of users is compared to a histogram of “clean EEG data” to calculate the probability that a PSD is from artifacts or from clean EEG data. The reason the method works for subtle artifacts is that those subtle artifacts are rare compared to clean EEG and so would have a low probability in the “good EEG” histogram.

### Calculating the PSDs

A signal is divided into sliding windows that overlap. For instance, a window 1 second long is used to calculate a Fast Fourier Transform that calculates the voltage magnitude and phase at each window. The window slides over 1/10 and a new Fast Fourier Transform is computed. Note that the next window covered samples that overlapped 90% with the previous window. The Fast Fourier Transform calculates what the Power Spectral Density of a window called V where each entry corresponds to a frequency with complex value of voltage magnitude and phase. The PSD is converted into decibel power to a matrix called P so that each entry is P_{f}=10*log₁₀(abs(V_{f})·̂2).

A histogram can be represented as a two dimensional matrix H_{fp}. The rows of the matrix are frequency bins and the column decibel-power bins. A frequency bin is a range of frequencies and a decibel-power bin is a range of decibel power values. The power at each time in decibel-power matrix P is added to its corresponding bin in the H matrix. A matrix H is built for each recording and will have the number of counts of decibel power per bin per frequency bin (i.e. a two dimensional histogram).

Using a database of EEG recordings from multiple users and across different study types (such as calibration and mindfulness), the spectrogram (i.e. a time series of PSDs) and a histogram H is computed for each recording. The histograms are built offline and are used to estimate the probability that a segment (i.e. window) of a signal is clean EEG.

Optionally, to select “good EEG data” in a first pass, the system may calculate signal variance to establish thresholds. High signal power is an indication of bad data. This first pass is used to eliminate obviously bad data. The sensor data is divided into overlapping window segments where for example each window is 1 second long and the windows slides in 1/10 second increments. For each signal (i.e. sensor data) window, the variance across the voltage values is calculated. The variance is calculated for all the windows in a sensor data stream. The threshold is calculated by taking the minimum variance and multiplying it by 5. If the voltage units of the sensor data are known then this threshold can be expressed as an absolute value of power in units of microvoltŝ2. After this threshold has been established then each window is marked as “bad” if it exceeds that threshold and “clean” if it is below that threshold.

Windows that are deemed to have clean data are selected and their PSDs, i.e. matrix P, are calculated. Each PSD is normalized before building a histogram in order to make the method independent of signal amplitude. The PSD per window is normalized by summing the total power across all frequency bins in that window in P and then dividing each entry in the matrix P at that window by the total power. These normalized PSDs are used to build a histogram of the power in each frequency bin of the PSD, i.e. 2-D histogram called H_{fp}.

A histogram may be built by accumulating data across all of the recordings in the database. Separate histograms per user may be built customized per user.

Each entry of the histogram H is the number of counts of a clean window per frequency and power bin. In other words, say there are 1000 windows of clean data. Each window has a corresponding PSD. The frequency and power ranges of the PSD are divided into ranges called bins. Each frequency bin has a corresponding power, for example frequency range 5 to 10 Hz for window #1 has power between 20 to 25 db of power. A count of one is added to the Histogram for frequency bin 5-10 Hz and power bin 20-25 dB.

A probability density function (pdf) is created from the matrix H. Pseudo count of 1 may be added to bins with 0 counts. A probability density function (PDF) from the histogram of counts is created and the log of the counts of each entry may be determined. The system may calculate the sum of these logs across the power per each frequency bin. The log of the power is taken to smooth the pdf and make its distribution closer to a Gaussian distribution. Each count per frequency bin is divided by the sum of the log counts. Therefore the sum across the pdf per frequency adds to one.

In order to classify a window in real-time, the system may compute a fft on the window of real time incoming sensor data the same way that it was computed when building the histogram (1 second window with hamming window). The PSD may be obtained by performing 10*log 10(abs(FFT)·̂2), smooth in the frequency domain, and moving average of 2 frequency bins. The probabilities of the real-time PSD per frequency bin are summed together to calculate the probability of being clean EEG. A probability threshold is applied to classify a window as clean EEG or bad EEG. Bad EEG windowed segments can be rejected from being processed as they are not representative of brain signal.

### User Flow

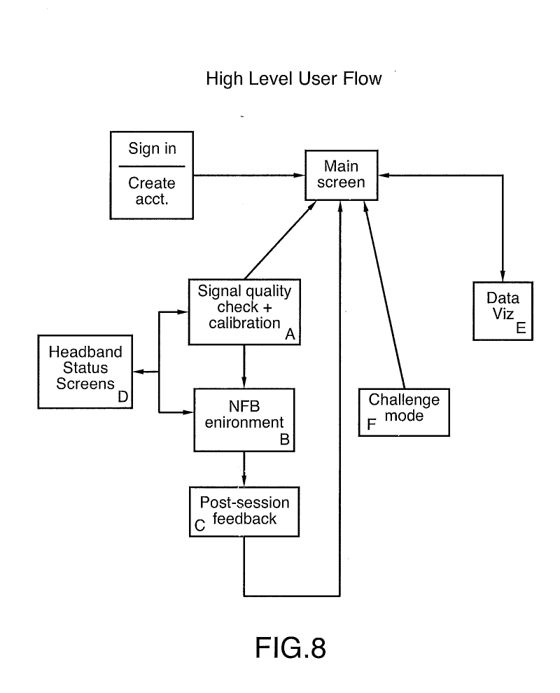

In an exemplary non-limiting implementation, a high-level user flow of the present invention is shown FIG. 8. The reference letters shown in these figures correspond to the descriptions found below. The User Flows also have connector boxes with a Figure label that show which Figure it connects with.

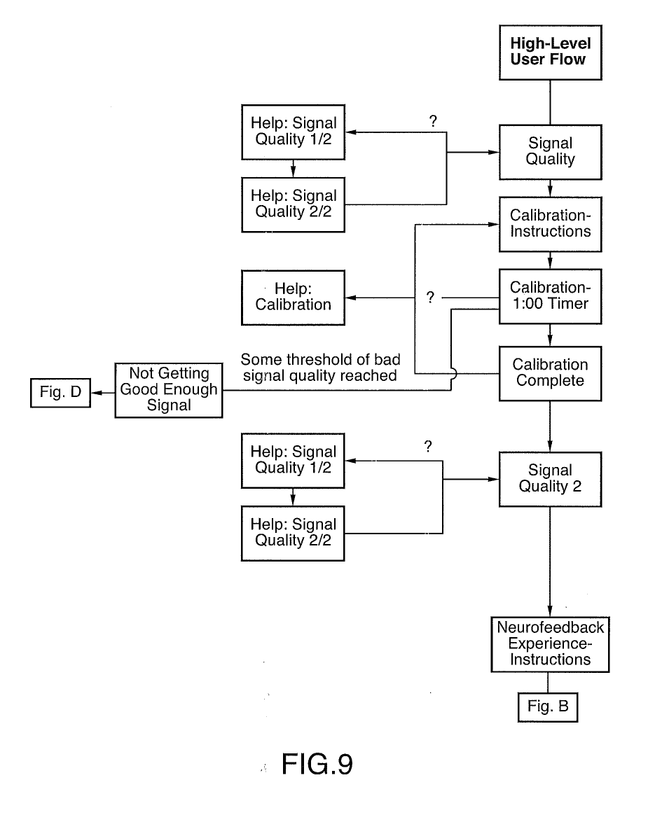

Before executing a brain state guidance exercise, the system may perform a signal quality check and calibration. FIG. 9 shows typical steps performed to perform a signal quality check and calibration. A solid electrical connection with low impedance between the electrode and surface of the skin is needed. It also needs to be free from a significant amount of artifact such as muscle contraction or eye movement that interferes with picking up a strong brain signal. Without a professional to perform a calibration, any self-administered brain signal implementation must make a strong effort to teach and encourage users to ensure good signal quality when training.

Whenever engaging with an Adaptive Brainstate Change Notification session, users will need to calibrate the system to ensure that feedback is provided to support an appropriate subset of all possible values. Calibration produces a range which matches the user's current context, a range which can be affected by a number of factors, including current brain state, skin conductivity, temperature, humidity, etc. After calibration, the system should have built a histogram on which two “goalposts” can be defined as the extremes of relevant neurofeedback (e.g. the busy-mind continuum described herein).

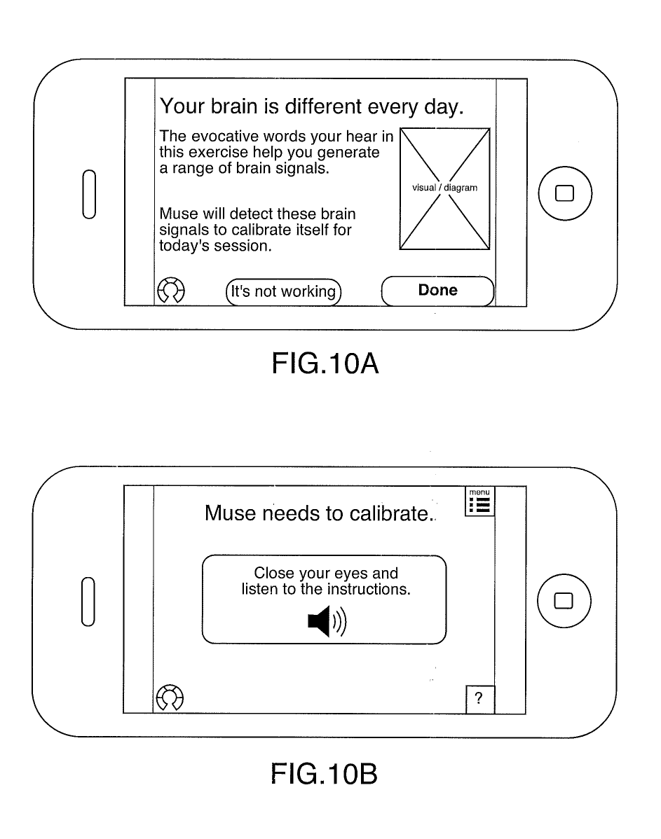

Even after distilling a complex process like calibration into something easily understood, users may still glaze over on-screen written instructions. Using verbal audio guidance may be more successful. The system may simply present a screen reminding a user to listen to the instructions provided. An audio icon may also include, in case users don't have the volume up. If users access the help screen, they may be presented with a short written summary of their instructions. Examples of some calibration screens are shown in FIGS. 10A-B.

Users may better to respond to calibration where the technical need for calibration is clearly communicated while soliciting user cooperation. The term “brain signals” may be used by the system as a clear expression of the complex idea of neurophysiological voltage coming from the brain and analyzed for different frequencies (as opposed to “brainwaves” or any other attempt at simplification). Users already know what a brain is, and they understand the concept of a signal, so the term is universal and self-explanatory. It also ties into our other screens where we discuss signal quality, noisy signals, good signal, etc.

The need for calibration may be explained to the user by the expression “your brain is different every day”. Using a metaphor of sensation may help to explain the need for users to be still and calibrate: “To calibrate itself, the system will take a snapshot of your brain in a resting state. This snapshot will be used as a reference to help the system understand your brain signals. For this calibration, the system will need to listen to your brain signals for 60 seconds.” Referring to the calibration exercise that users have to do as “a simple task” may help them understand its arbitrariness and kept them at ease during the process.

Users may assume awkward positions when calibrating and engaging in neurofeedback exercises. This problem may be solved by including suggestions for users' physical position: “Sit in a comfortable position. Allow your back to be straight and relax your shoulders.” This also contributes to good signal quality, as movement and tension causes noise and artifacts in the signal. To avoid the user clutching the computing device during calibration, the system may direct the user to put down the computing device. Viewing a display of the computing device may also not be required during calibration.

Just before the calibration begins, the system may add a final reminder about signal quality and noise to solidify users' understanding based on previous signal quality screens: “For a good calibration, try not to move too much. Noise makes it hard for the system to hear your brain signals.” This not only reminds them to be still, but also sets the user experience up for the case where bad signal quality forces an error.

One example of a brain state guidance calibration exercise includes directing the user to imagine a perfect day, then imagine the next 24 hours, then imagine the past 24 hours. Another example may include a categories game. Users may be engaged with the task, and its arbitrary nature may help the users' understanding that the calibration was not part of their self-administered training, and instead more of a technical need. This exercise may generate eye-movement artifacts, which aligns with psychological research correlating eye movements and memory retrieval. Another example may include an exercise to visualize a dot. This exercise may work well for some, but others may have difficulty visualizing the dot and staying on task. Even for short periods, users may become bored with this task. Another example may include emotional words. Users may be engaged by this exercise, but it may also change the users' interpretation of the ABCN training in general. The use of emotionally charged words may lead users to falsely believe that the training was about emotion. Another example may include Body Scan/Breath focus. Calibration exercises that are similar to the main brain guidance exercise may lead to a poor user experience. The similar exercises may bleed together and cloud the user's understanding of calibration in general.

While the traditional and easily understood method of calibration is to have users perform a calibration exercise while sitting still for 1 or 2 minutes, this approach may be problematic. The system has no guarantee that users will provide enough good quality data to calibrate, and so precautions need to be taken to maximize signal quality. A more consistent and effective approach is to use a “gastank” analogy. The user interface communicates that a certain amount of good signal is required to proceed. When users remain still with closed eyes and their data is free of artifacts, the user's “gastank” progressively fills (with both visual and audio indication). Whenever users open their eyes, move, tense up, or lose connection with the headband, the “gastank” stops filling (and audio/visual indication reflects this). This ensures consistency across users, guarantees an appropriate amount of calibration data, and motivates users in an effective and amusing way. Subtle audio feedback may be provided by the system to emphasize good data. Calibration exercises may involve the user sitting still with closed eyes and performing a relatively mundane exercise for over a minute. In order to maintain engagement and provide a subtle feedback, the system plays a gentle a pleasing sound when good data is being received. This sound fades out when artifacts or other signal quality issues are present.

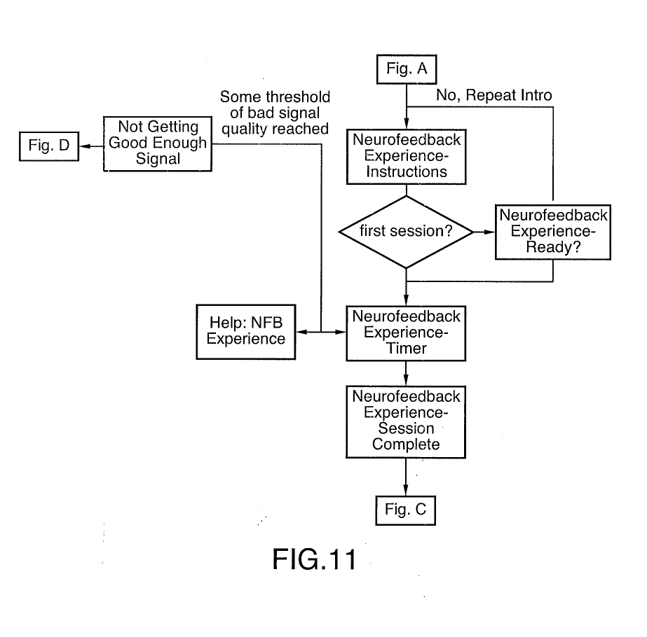

Once calibration is completed and the headband signal strength is good, an ABCN session with a brain state guidance exercise may commence, as shown in FIG. 11.

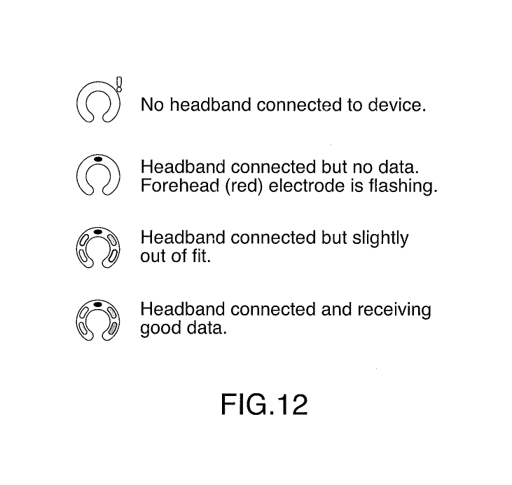

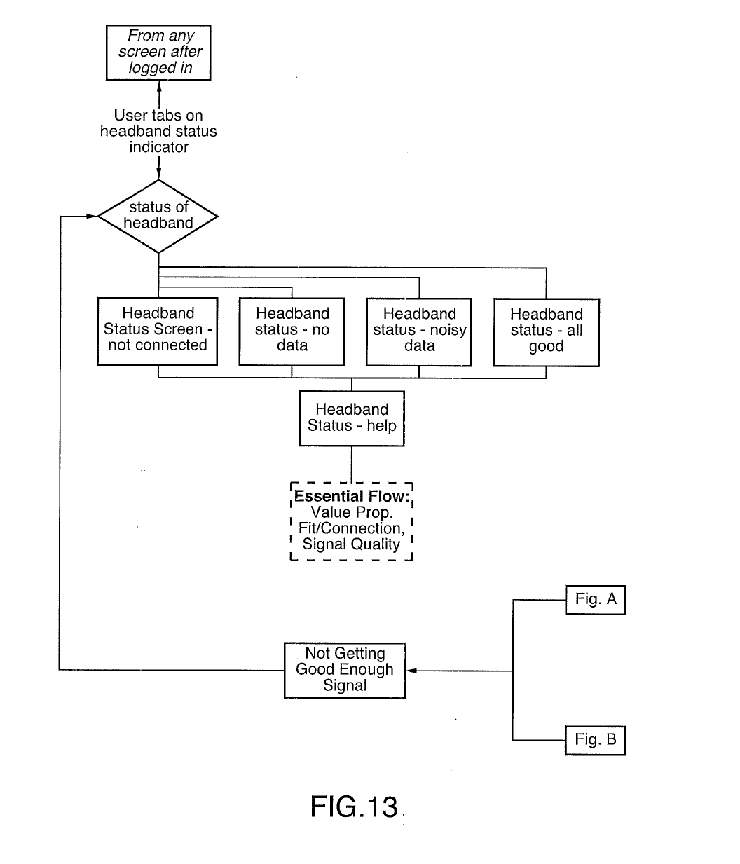

It is important that the user is notified of the status of the headband fit providing the brain sensors and the connection. The computing device may provide an indicator that communicates the status of the headband fit and connection to the user at all times when a user is signed in. The indicator is shaped as a simplification of the headband's shape, allowing users to spatially determine their headband fit and connection problems. Individual regions of the indicator correspond to electrodes on the headband. An exemplary indicator is shown in FIG. 12 showing various states of the status indicator. FIG. 13 shows typical steps performed to provide headband status to the user. If the headband is not present or connected to the device at all, the entire indicator is outlined and red to communicate an absent headband. If the headband is connected but not receiving signals from a user's brain the indicator is white showing the connection but lack of signal. The region of the indicator which spatially identifies the ground/reference electrodes is also flashing red to communicate that either the headband is not on at all, or the special ground/reference electrodes are not making contact (preventing signal from any electrode). Since the system can't differentiate these two states, this visual draws the appropriate response from users in either case. If the headband is connected and receiving signal, there are independent regions of the indicator which spatially tie to each of the headband's electrodes. Each electrode's visual region can be blank (no signal), outlined (noisy signal), or filled (good signal). The region of the indicator which spatially ties to the reference electrodes is black to reflect the solid connection. With a relatively short explanation of the headband status indicator, users are able to articulate it's meaning, and often respond fairly appropriately to signal quality problems.

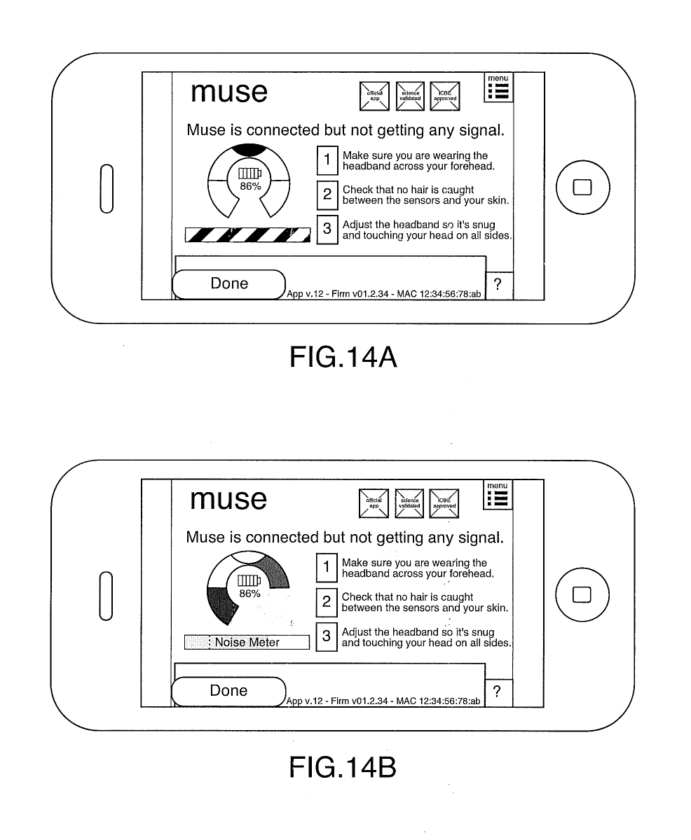

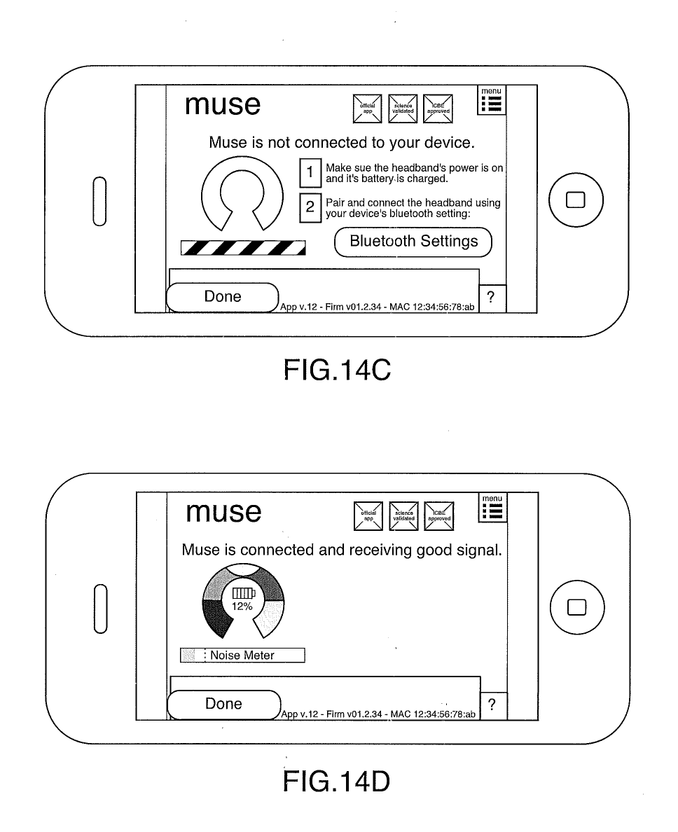

At all times when a user is signed in, the user can tap on the headband status indicator screen to get additional detail. The screen includes: Live brainwaves to reassure user that the system is responsive and connected; A larger version of headband status indicator to help learn and understand it; A clear status update which communicates the current status of the headband and updates in real-time; 3 tips to solve any problem with the headband which update and change in real-time based on the current status of the headband; An algorithm to control status/tip update to prevent it from being volatile (e.g. user has to demonstrate good signal consistently for 5 seconds before the status update and tips reflect that; or user has to demonstrate noisy signal consistently for 5 seconds before the status update and tips reflect that); A battery indicator positioned within the headband status indicator showing how much battery is remaining; and A help link to replay the same interactive presentation/video discussing headband fit/connection which all users saw when they created their account. FIGS. 14A-14D show various states of the headband status screen.

The system may be configured to always provide a signal quality check screen prior to calibration or the commencement of an ABCN brain state guidance exercise. Whenever users are about to begin an interactive session involving the headband, they are presented with a signal quality check screen which ensures that the headband is ready and that the user is relaxed and still enough to get started. The screen includes: a large headband status indicator to communicate the current status of the headband; a noise meter which fills up from “ok” to “noisy” when artifacts are detected, showing users their current signal overall in real-time; a 5 second countdown timer which counts down good signal and resets when bad signal is detected. The timer is not reset unless bad signal is detected for over 200 msec, in order to skip blinks; a written “tip” which appears when signal is too noisy, reminding users to check their fit and relax; a message that confirms that the headband is ready after 5 seconds; and a help screen which instructs users to try all kinds of muscle movements and see how it affects the noise meter in real-time—this screen defaults as visible the first time a new user uses the system.

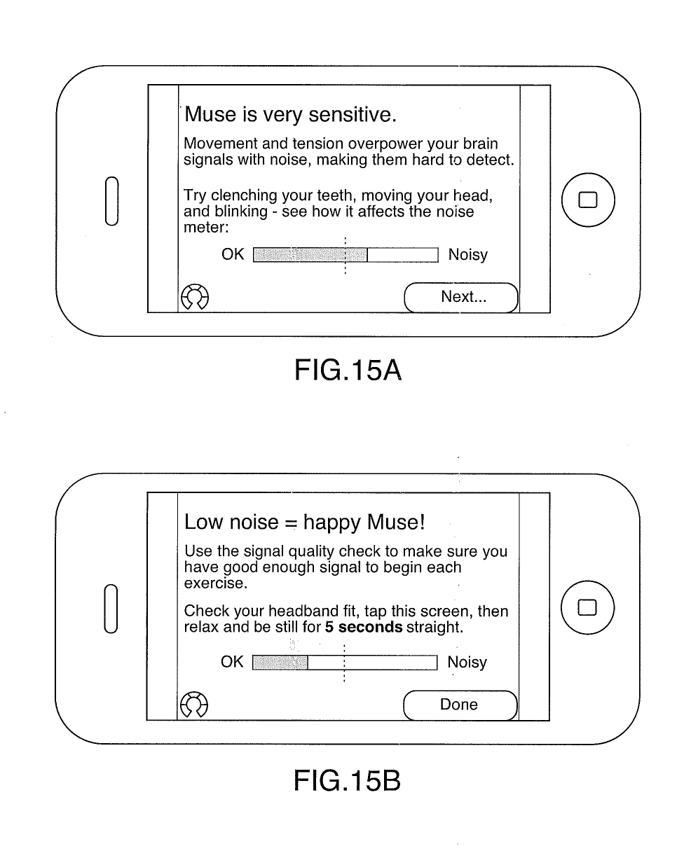

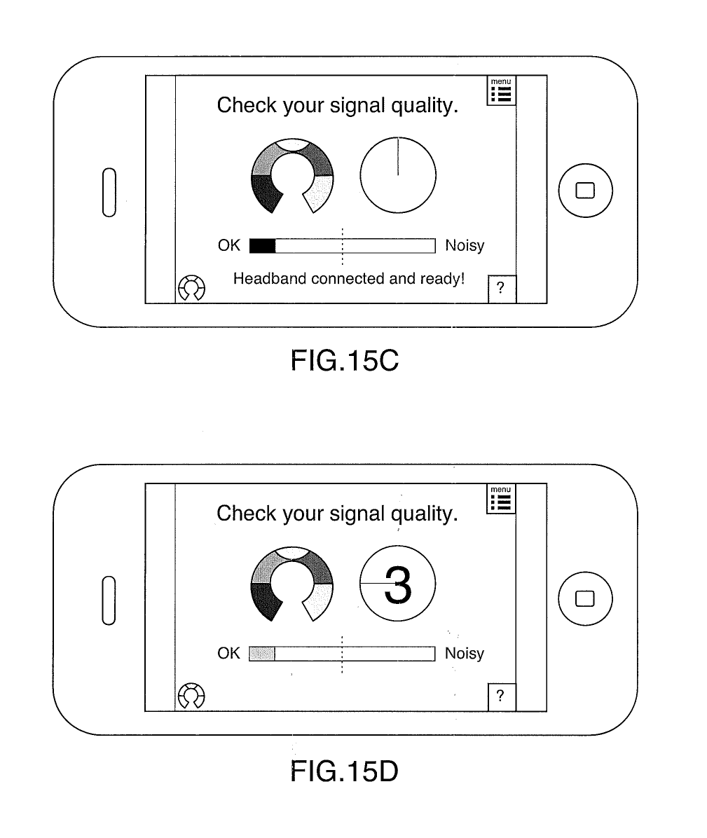

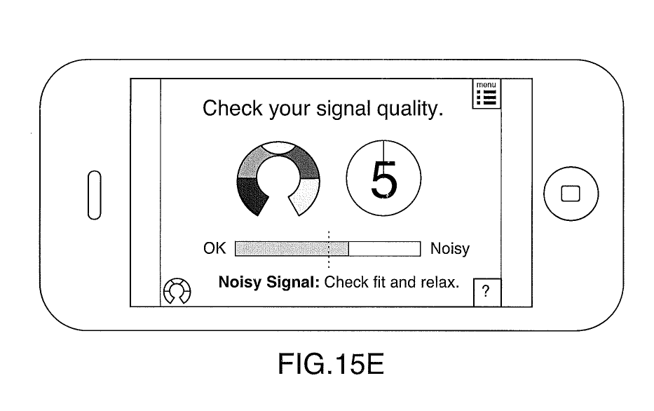

FIGS. 15A-15E show various states of the signal quality check screen before calibration. While a “noise meter” may provide a technical framing, as well as having the proper inversion of quantity (e.g. when users grit their teeth or move, they bar fills up showing the brain sensor being overloaded with signal), it may be unclear to some users. In order to effectively communicate signal quality, it may be effective to communicate to users about the state they are not in (e.g. when their signal is good, it's important to tell them that it's not noisy; when the signal is noisy, it's important to tell them that it's not okay), and therefore label each extreme in the meter shown in the Figures.

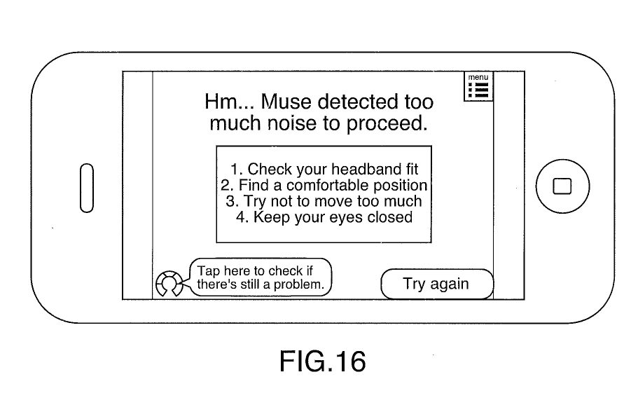

The user experience and efficacy of self-administered ABCN will be drastically affected if there are signal quality problems. For this reason the system may implement signal quality alerts which are triggered when bad signal quality overwhelms the system. During calibration, artifacts are especially problematic, as they can change the behaviour of the session and have disastrous effects on the user experience. So during calibration, if the system picks up a cumulative total of 10 seconds of bad data, the session is arrested and user is asked to check signal quality and restart. During the ABCN session, it can be a bad user experience to simply kick a user out without warning and force them to restart. A two-tiered alert system was devised to solve this problem: (1) cumulative total of 10 seconds of bad data: system plays chime along with a verbal recording of the narrator explaining the problem; and (2) cumulative total of 20 seconds of bad data: system plays the chime and arrests the sessions and asks user to check signal quality and restart. FIG. 16 shows an example of a signal quality alert during calibration or during an ABCN session.

### Brain State Guidance Indication Metaphors

Since ABCN is a complex process, traditional approaches tend to be handled by experts. By integrating the ABCN experience into a metaphor ontology which maps to the training, users can be engaged deep enough into the process to self-administer. A “metaphor” in ABCN as employed by the system of the present invention may be a particular mental task to focus on that the system has associated with producing or achieving a particular brain state response in the user. For example, the system of the present invention may aim to help a user manage stress and calm/settle the mind using a particular UI element or scene that the system has associated with the desired brain state. When the system determines that the user's brain state is closer to the desired brain state, the scene may appear richer, calmer, or more in focus. When the system determines that the user's brain state is further from the desired brain state, the scene may appear noisier, more chaotic, or more out of focus.

For example, weather or wind may be used as a metaphor for a calm mind in the present invention. The user may be presented with a calming scene, such as that of a beach or other vista with a representation of blowing wind, either visually, or aurally, or both. As the user's brain state is determined to be closer to the target brain state, the intensity or volume or any other indication of the blowing wind may be reduced. Users may therefore understand their task of calming the winds, and implicitly learn the depth of the metaphor that just as weather ambiently affects your day, so does your natural brain state. This way users don't have to learn about EEG frequencies or anything too scientific, but simply train their ability to “calm the winds” which translate to the real-world benefit of calming and settling the mind.

In another example, for achieving a focused mind brain state, a metaphor employed by the system may be that of a target. The target may come into focus when the system determines the user's brain state to be closer to the target brain state of a focused mind, and the target may go out of focus when the system determines the user's brain state to be further from the focused mind state.

The term “metaphor” as used herein means a representation used as a language for inducing, instructing, interacting with and communicating about brainwave technology and experiences with users. Like the metaphor of “desktop” and “mouse” was created and standardized as a metaphor for interacting with content on a screen, in accordance with an aspect of the present invention, a metaphor for how a person may use and think about brainwave technology, and brain-computer interfaces is provided. For example, using a metaphor of weather may help a user to relate to the level of activity of the user's brain, without the user having to actively think about being calm or being agitated. Instead, representations of weather graphics or sounds may be presented to the user in response to the user's measured brain state, such as the system presenting a wind sound or graphic when the user's brain is agitated, and presenting a bird song graphic or sound, with no or reduced wind, when the user's brain is calm. This weather metaphor may help the user to relate to the level of activity of the user's brain.

The system may be configured to present a representation of blowing wind when the user's mind has wandered (lacking stability). The system may direct the user, through a brain state guidance indication to focus on counting the user's breaths throughout the exercise. The blowing winds may remind the user to come back to counting their breaths.

In accordance with aspects of the present invention, 5 major metaphors for the human mind and its relationship to a brain-computer interface have been determined. These metaphors include: Musical metaphors like [self=instrument], [mind=orchestra] and [meditation=tuning]; Water-based metaphors like [mind=water] and [distraction=waves]; Improvement as cultivation based on votes distributed across metaphors like [brain=garden], [mind=soil], and [neglect=decay]; A pursuit of self as destination with metaphors like [self=destination], [improvement=journey] and [travel=transformation]; and A meteorology metaphor where [mind=sky], [body=earth] and [distractions=gusts]. These are all different relatable ways for the user to think about the ABCN system which may simplify technical details of brain state training and guidance, allowing the user to engage deeply. While the wind metaphor may be described herein, other metaphors may also be implemented in implementations of the present invention.

Optionally, musical metaphors for mind may be inserted into each problem dimension identified for our system. The “tuning” metaphor may be an effective conceptual metaphor for the system of the present invention in some ways, especially in what it reveals about the mind. However, the musical metaphors may fail in more practical dimensions. In the visual design, headband setup, calibration, gamification, and curated data dimensions, musical metaphors may not be effective. Further, music may be open to differing interpretations from different people.

With respect to a mind=water metaphor, it was determined that water has a lot of shared entailments with meteorology. A shared conceptual space between water and wind (which may be called mind=fluid) may have overlap and shared entailment, as well as comprehensive coverage across the problem dimensions. Mind=fluid has a strong ability to facilitate the use of simple and universal language to describe the mind to anyone. It's universality around the planet offers a potential for being non-judgmental (waves are not inherently bad, neither is strong wind). It has very pleasant potential for visual design and an aspirational brand while revealing the complex, turbulent and everchanging element of mind. It does a good job at hiding technology/science/machine views of mind while still being functional and useful for brain training and calming oneself. It has a strong flexibility for multiple experiences and audiovisual richness.

While water has a lot of great entailments and subtleties to the experience, its potential for audio richness is lacking compared to gusts of wind. In this and many other ways, the two metaphors complement each other perfectly with shared entailments. Optionally, the central shared entailment may be built out as brain=atmosphere. This represents a clear and permanent physical metaphor for the physiological element of brain, while the transient notion of mind can equate to both water and wind as transient fluids in that physical space. While weather/wind as a metaphor for mind may be generally preferred, thunder and dark clouds may be viewed negatively. The brain=atmosphere approach does not include the “storm”, but instead occupies the space between gusts of wind and waves.

Accordingly, the wind metaphor may best provide a suitable method for communicating brain state guidance to the user. Other weather conditions, such as dark clouds or thunder may be viewed negatively by the user, and are less suitable than wind. A technical implementation of wind sounds or animated visuals over water sounds or animated visuals may be more relatable to the user. Accordingly, metaphor choice may play a major role in the explanatory mechanisms of the present system. Instead of explaining brain science to the user, the system instead attempts to explain how the user's relatable mental states compare to audio and visual feedback consistent with the chosen metaphor.

Considering the self=destination and improvement=cultivation directions, neither of them were able to get expansive coverage of the problem dimensions on their own due to their conceptual nature. While these metaphors reveal very interesting things about the mind and the process of using the present invention, aligning them with problem dimensions of self-administered neurofeedback may not be effective. Practical concerns like visual design, aspirational brand, headband setup, calibration, etc. are very hard to align with such conceptual ideas. There is no easy visual approach nor easy way to provide explicit guidance or curated data.

There may be a contradiction for self=destination and improvement=cultivation in the context of the brain=atmosphere direction. If brain=atmosphere, mind=fluid, and distractions=gusts/waves, then the focused, stable attention (which is the goal of the app) is not about going anywhere. In fact, the successful user is staying still in a peaceful atmosphere. This aligns much better with the cultivation model, where a calm mind is a calm atmosphere, and thus things are allowed to cultivate and arrive. With the destination model, progress would mean moving somewhere, which is in direct contradiction with the stillness of success in the brain=atmosphere entailment.

Optionally, music may be used as a separate metaphor with which to engage other users of the present invention in real-time (e.g. other mind=instrument). This is largely an aesthetic decision, as using music to represent interconnection with other users may provide for “Journey” and “Ocarina”-style interaction between users.

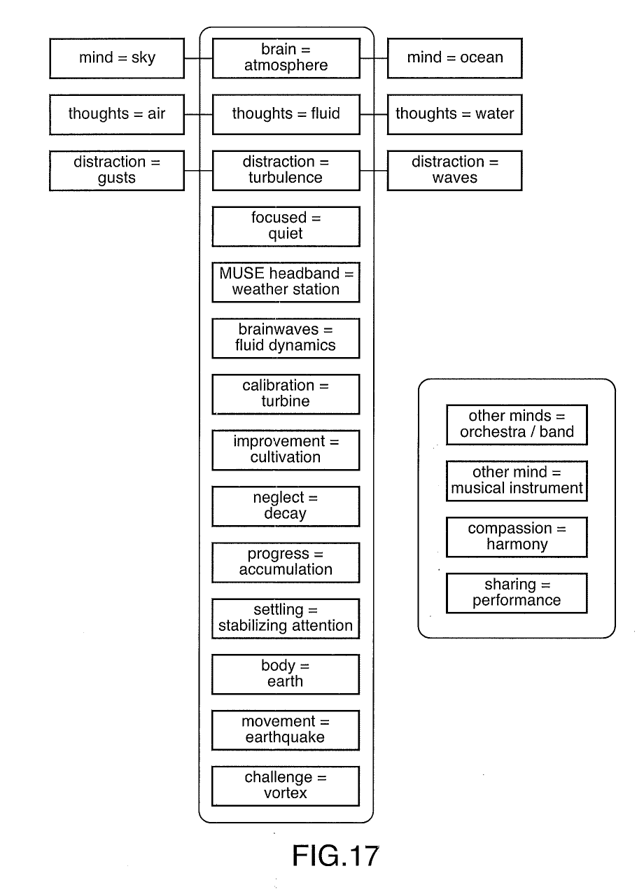

A non-limiting metaphor ontology is shown in FIG. 17. Implementations of the present invention may implement some or all of the shown metaphors in respective brain state guidance exercises.

### Brain State Guidance Audio Indications

As eye movements create artifacts, possibly due to unintentional minor eye movement, the solution for ABCN experiences is to use other modalities. Audio may be useful for ABCN as audio may offers an advantage of connecting with users in a way which can be peripheral to attention.

The use of audio samples which fit the metaphor of the system is important. As the main feedback paradigm, these sounds will be the key ingredient in teaching users about the metaphor.

Using an audio element which facilitates smooth transitions is important for the system to feel responsive—users need feedback not only for when they are in the target state, but at all times they should be made aware their distance from the target state on some understandable dimension. Smooth transitions allow the quality of feedback to implicitly communicate this information in real-time.

Users who perform well in the system of the present invention may tend to feel that the system is not responsive. The reason is that when training, high performers tend to hear a static soundscape, as their brain signals are not fluctuating a lot. In an aspect, the present invention may solve this problem with the idea of “emergent audio properties” which serve as rewards for high performers. For example, there may be provided additional audio feedback which rewards users who remain in the target state for consistent periods of time; and there may be provided a second level of extremely rewarding audio feedback which rewards users for rare moments when they move beyond the target state for consistent periods of time.

As ABCN training can offer subtle changes, it is important the audio feedback is mapped to emphasize changes. Users respond to clear changes more engagement than subtle variations. The “goalposts” of the calibration data should be optimized to maximize large peaks and valleys in users' data.

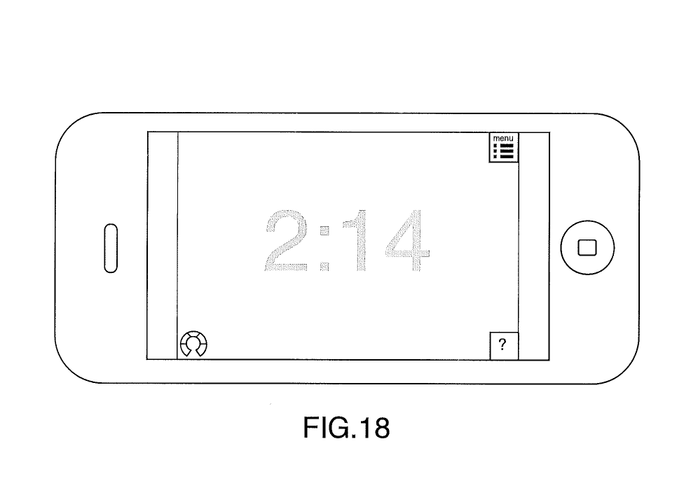

Audio feedback which reflects the negative state (the opposite end of the spectrum compared to the target state) should not be too judgmental about themselves. Users may respond negatively to a correlation of a negative state with very abrasive sound. That is not to say the sound should be pleasant, however. Users may engage readily with a subtly abrasive sound which communicates the negative state as undesired without creating too much self-judgment. The fact that these sounds reflect a user's brain state suggests that the quality of the sounds should be selected very carefully. FIG. 18 shows an exemplary screen view of an ambient visual experience.

Although users are encouraged to close their eyes and focus on audio feedback, the use of an ambient visual experience may help to frame the use of metaphor. An animation which is not too engaging, with no elements in the foreground, may evolve users' experience of a soundscape from being simply “interactive sound” to being more of a “world” which they inhabit during the session. When the animation is implemented, users may be more likely to connect with the “world”. An example of an ambient visual experience may include an ambient visual of a beach environment behind a session timer.

Calibration data can be shifted to alter different users' constantly changing needs for different difficulty levels. When users calibrate, their “goalposts” can be shifted by a certain percentage to change the difficulty of the session. If a user wants an easier mode, the goal posts can be shifted further away from the target state. If a user wants a bit more of a challenge, the goal posts can be shifted toward the target state.

As words only go so far to explaining feedback, but overly long explanations grow tedious, it may be preferable to implement levels of instruction in the app of the present invention. In one implementation of the system of the present invention, three levels of instruction are implemented in the app. Optionally, another guidance method may be provided that adapts to the amount of time a user chooses to train with. Prompts may be inserted into the duration of training to remind/motivate user to stay in-state. For example, in a 5 minute session, there may be 5 prompts (one per minute). In a 10 minute session, there may be 5 prompts (one every 2 minutes). In a 15 minute session, there may be 5 prompts (one every 3 minutes). Other quantities and distributions of prompts are possible.

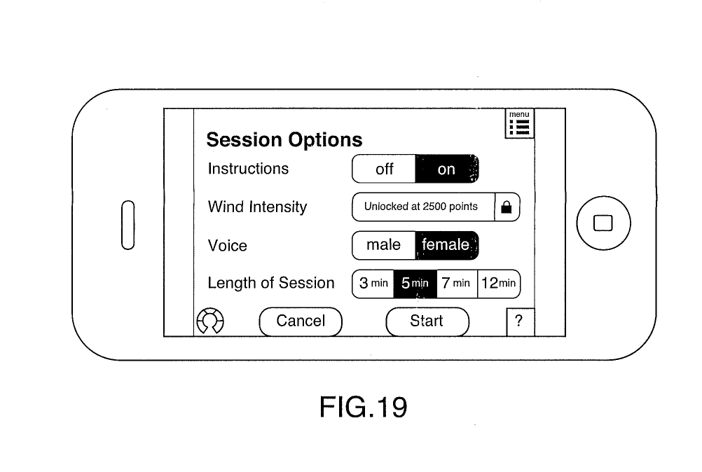

Special “first session” audio and visual may be provided to demonstrate extremes and explain the ABCN which introduces the full warm-up guidance flow. The user may be presented with an option to take the special “first session” tour. Optionally, the system of the present invention may be configured to automatically provide more detail on a new user's first session with the system. The application may be configured to set instructions “on”, thereby playing a full warm-up guidance flow to ease users into the calibration and ABCN experience. Instructions “off” condenses instructions into a concise single sentence which triggers more experienced users' memory of what to do without taking too much time. FIG. 19 shows an exemplary session options screen.

Even after distilling ABCN into something easily understood, users may still tend to glaze over on-screen written instructions. This problem may be solved by using verbal audio guidance instead.

Some physical positions of the user when calibrating and engaging in ABCN exercises may result in a better quality signal being received from the user's brainwaves. The system may provide suggestions for the users' physical position (e.g. “Sit in a comfortable position. Allow your back to be straight and relax your shoulders.”). The suggested physical position may contribute to good signal quality, as movement and tension may cause noise and artifacts in the signal.

During calibration, the user does not have to hold the computing device or look at the screen of the device. The system may be configured to provide a logical flow to users in order to guide the users' focus. First, since they are already paying attention to the device, the system may guide them to put it down or rest it gently (as mentioned above). Next, the system may instruct the user's body, advising them to find comfort and sit straight (as mentioned above). Next, the system introduces the exercise.

In a non-limiting exemplary embodiment, the app may be configured to direct the users to draw attention to their breath to encourage them to take hold of their focus. The app may also be configured to introduce the exercise of counting their breaths, a proxy for holding a focused attention on the present moment and avoiding distractions.

As people may be quite sensitive about their brain and mental performance, the app may be configured to be sensitive to this. For example, the app may be configured to remind the users, prior to a session, to not get wrapped up in negative judgmental thinking (e.g. “When you notice that your mind has wandered or you have lost count, don't worry. Just start back at 1 and resume the count again without judging how you're doing.”).

While journaling and self-report often plays a role in neurofeedback as provided by a professional, it may not engage users who are self-administering ABCN. Gamification of self-reporting may make it more engaging to users. The user may also be asked to label an entire session or time-specific parts of a session, or the user may categorize events in brainwave data as being either relevant or non-relevant to the exercise carried out during the session.

Sharing comments and progress with other people who are involved in the training paradigm through a social network might be appropriate and encourage more community engagement.

The system of the present invention may also be configured to allow the user to dedicate a key pivotal moment in a session, or the achievement of a milestone to an individual via email or other mode of communication.

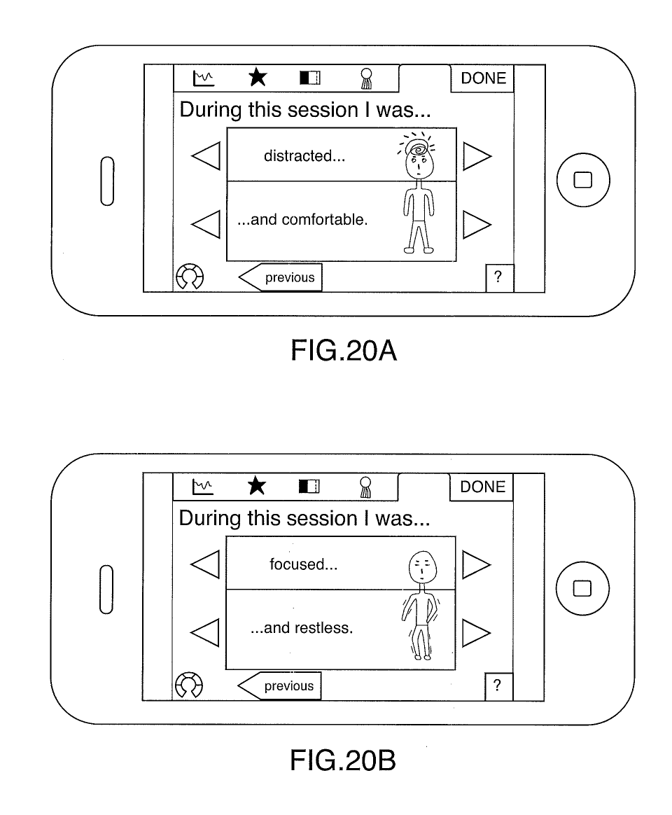

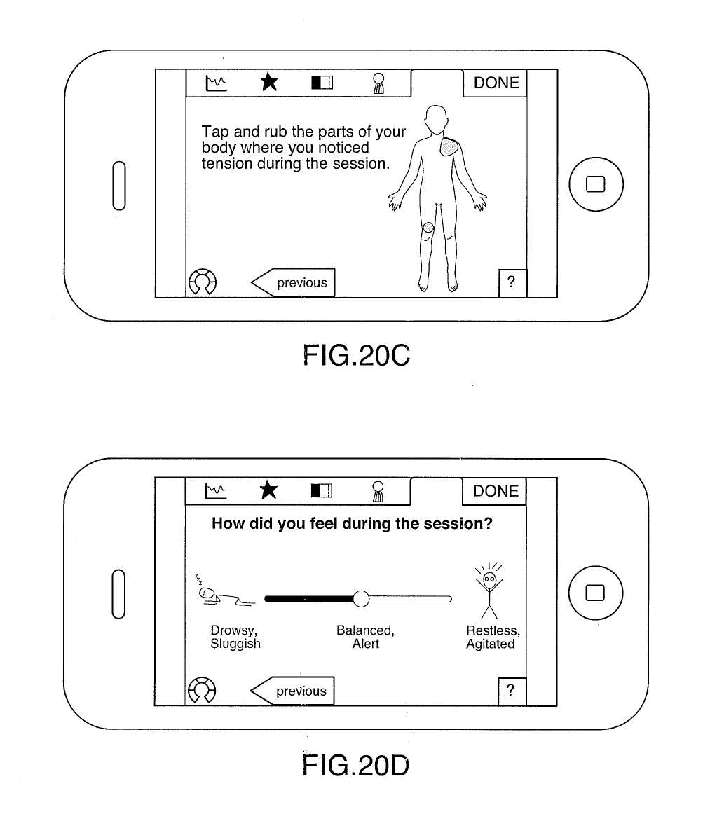

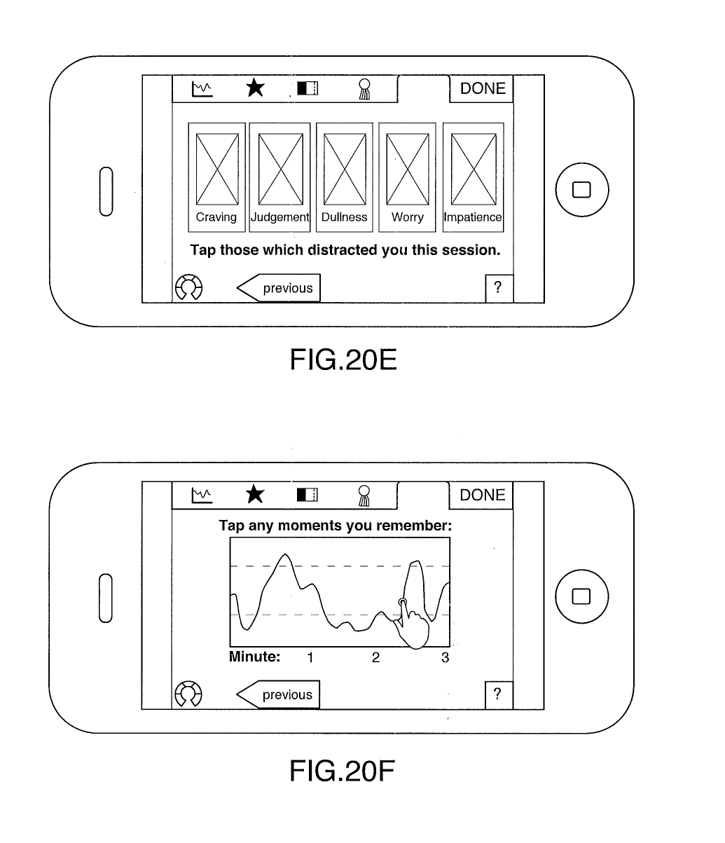

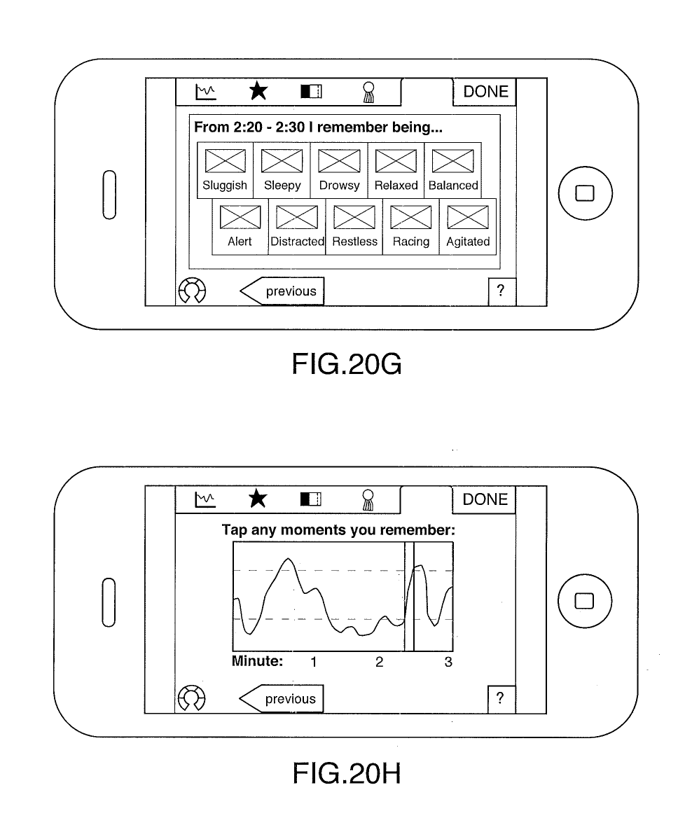

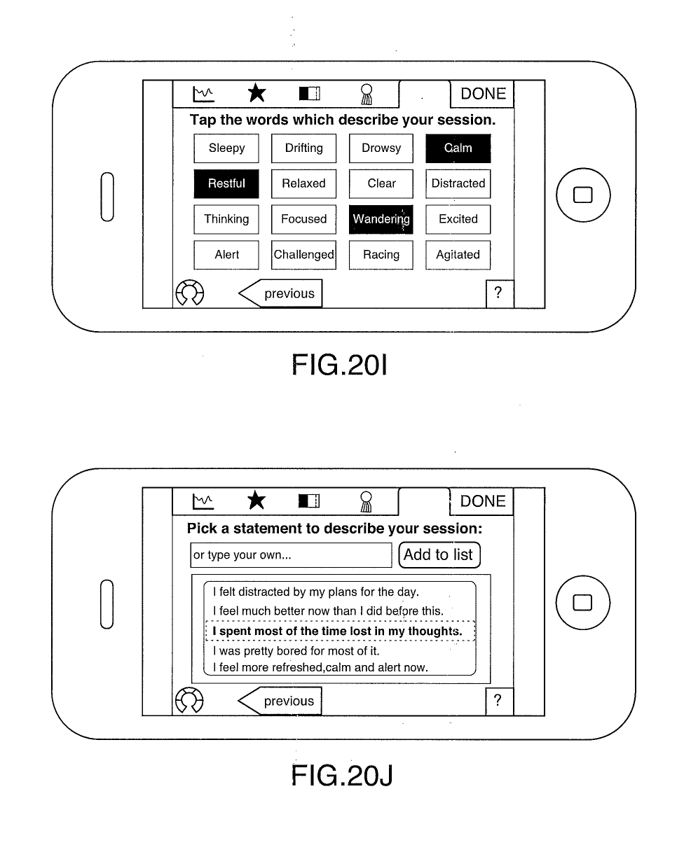

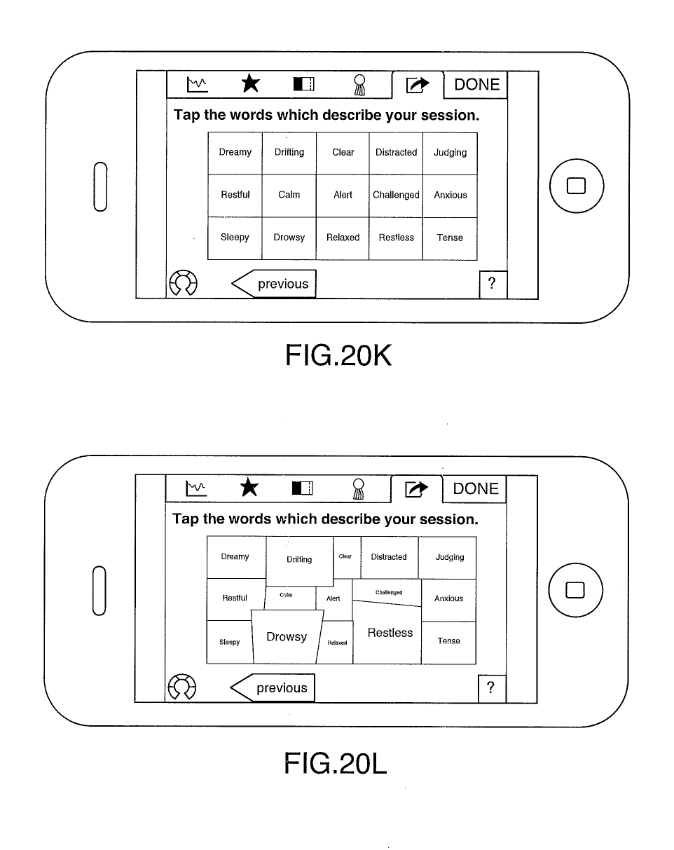

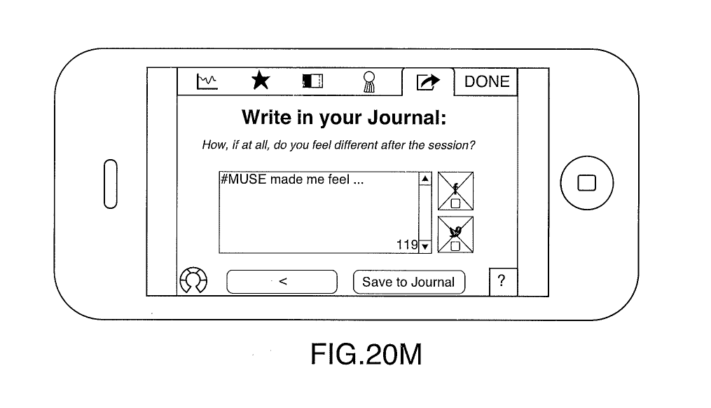

FIGS. 20A-20M show screens that may be used to solicit and receive a user's self-report. Self-reports, or user-response classification, may be used by the system to label the entire session and or time-specific parts of a session that can be used to provide labelled EEG data for machine learning applications.

### Brain State Guidance Exercise User Interface Embodiments

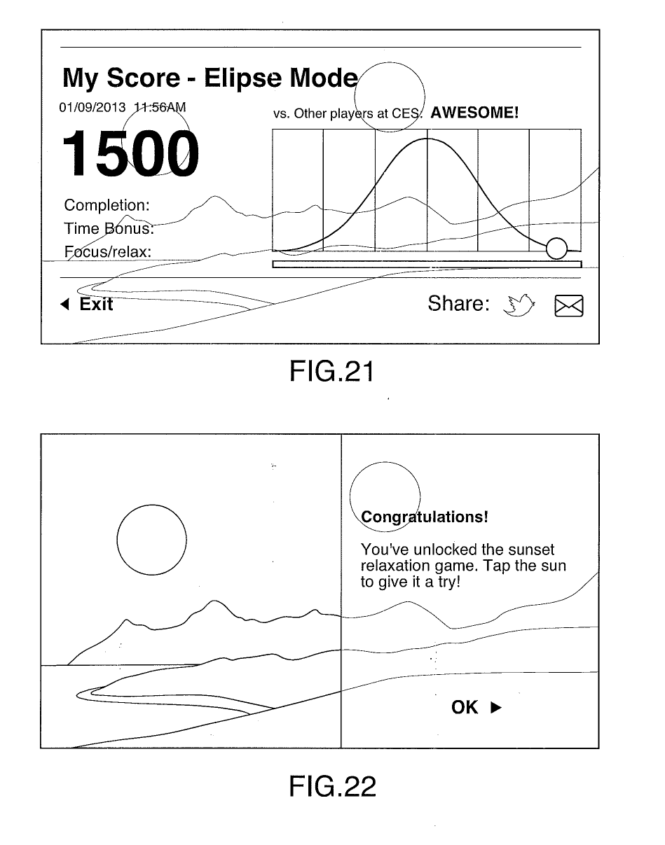

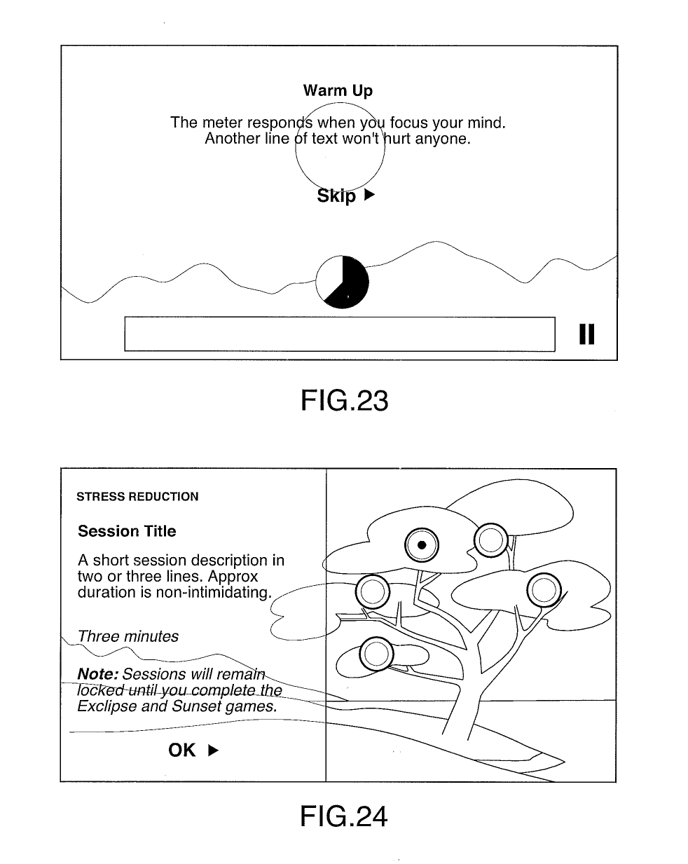

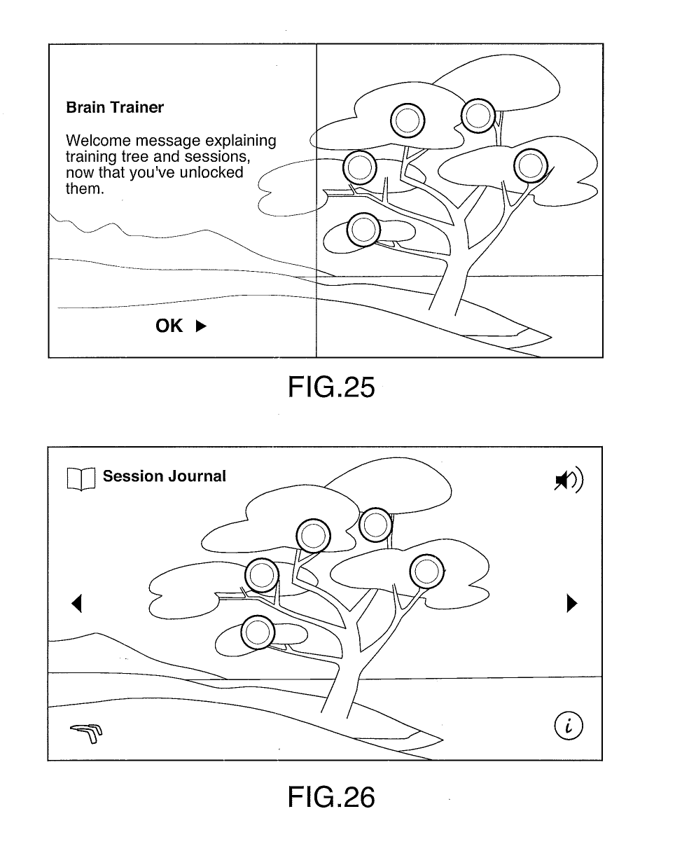

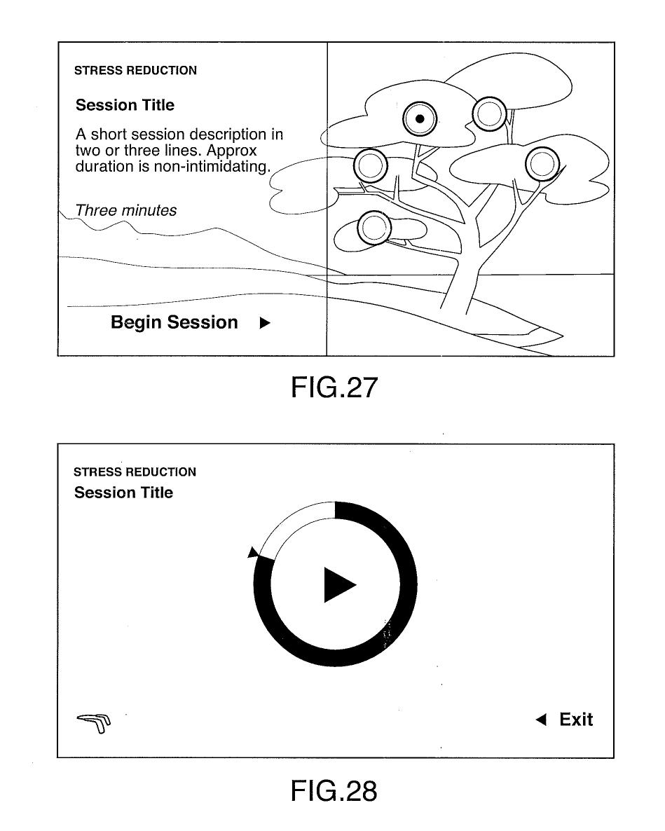

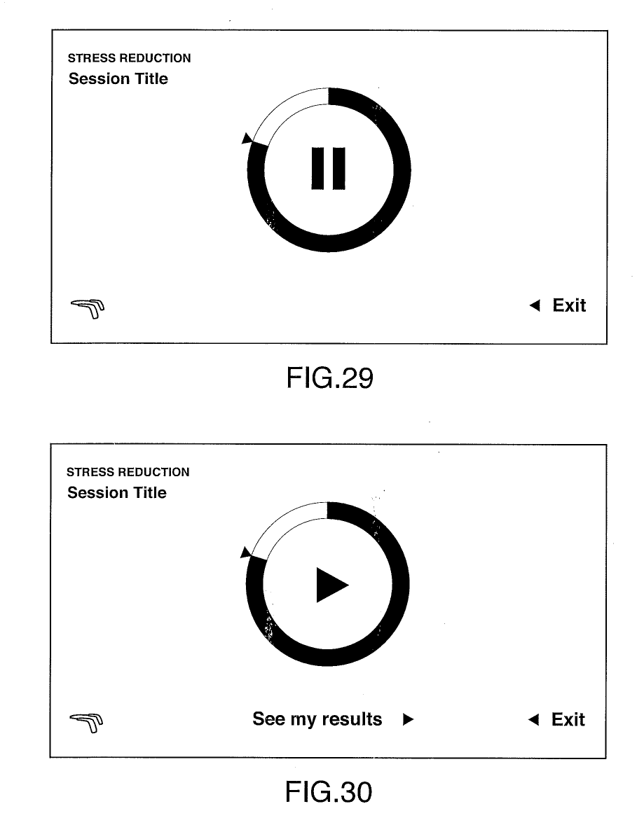

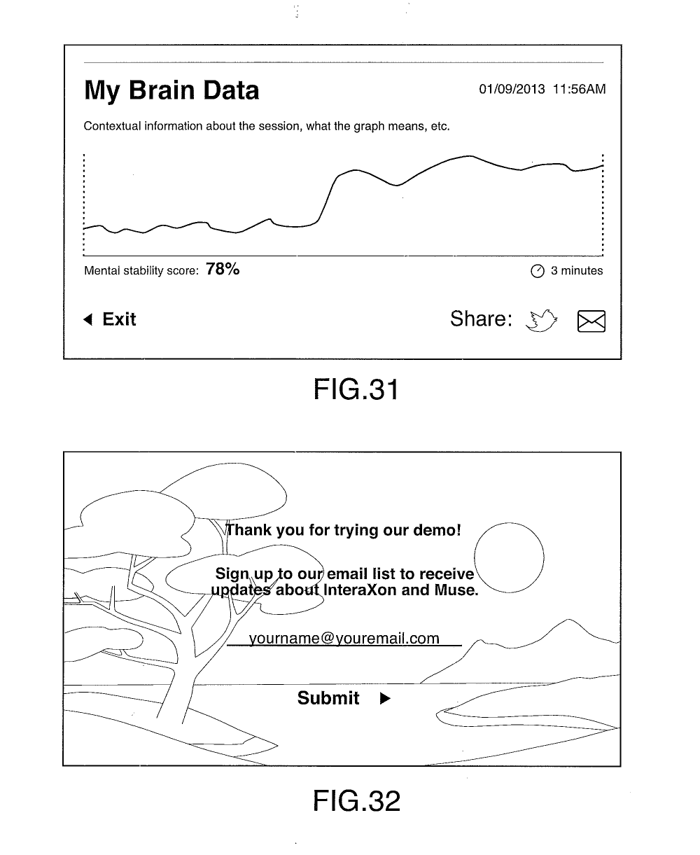

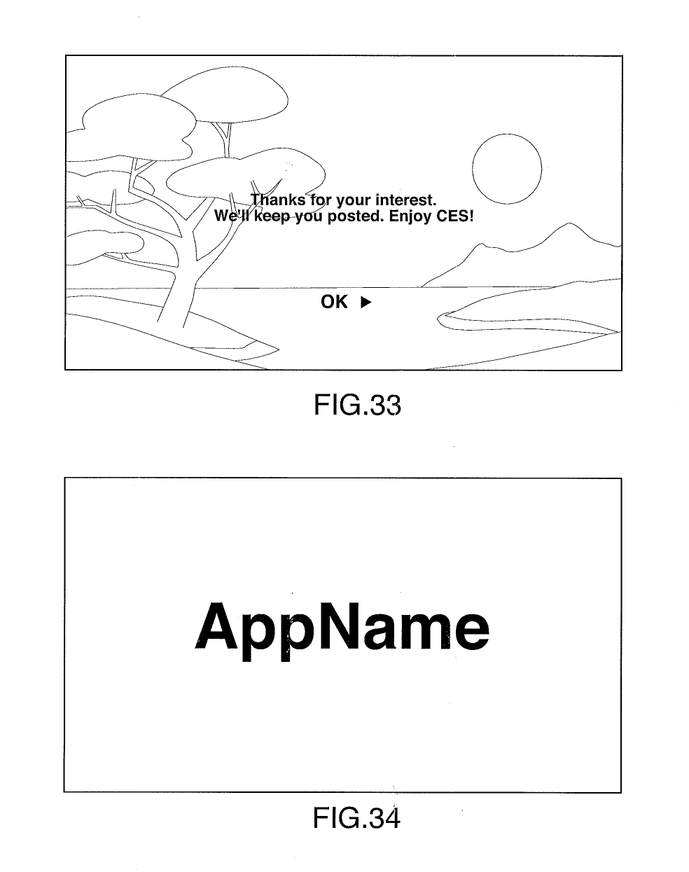

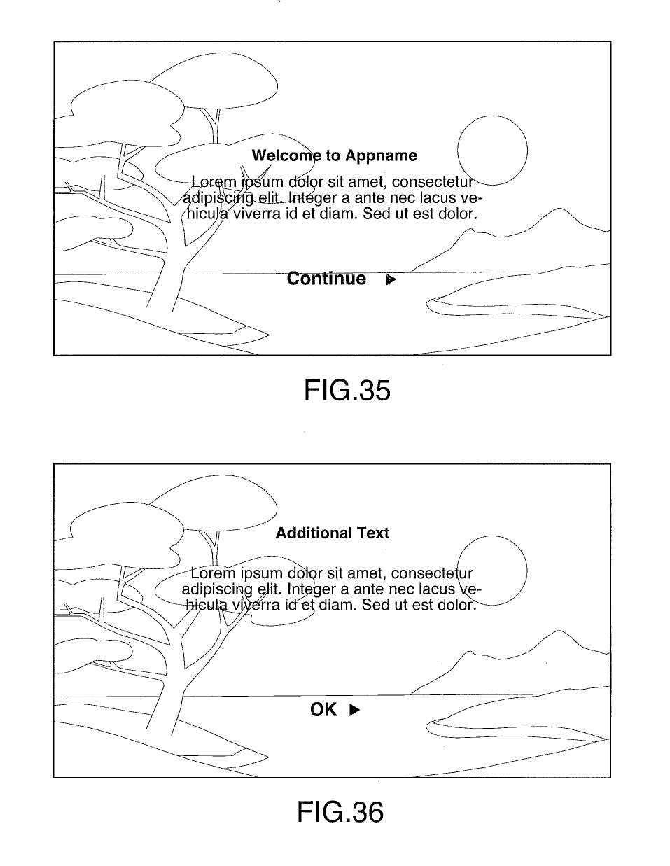

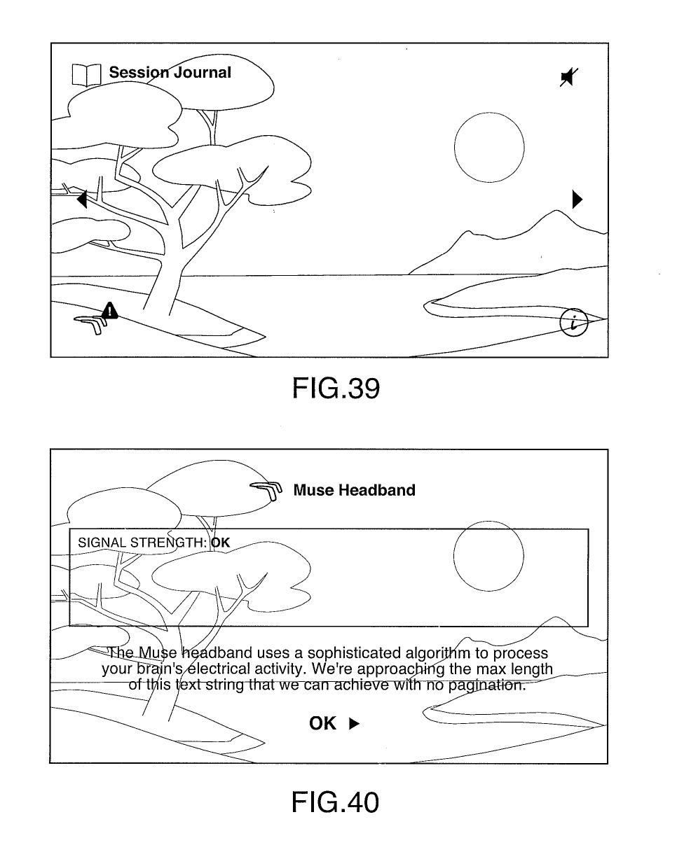

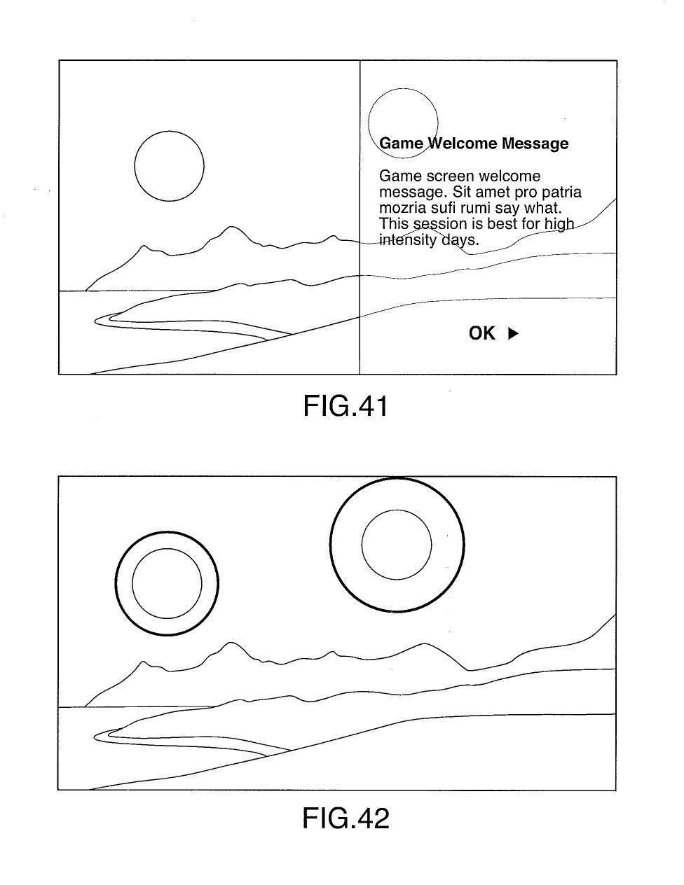

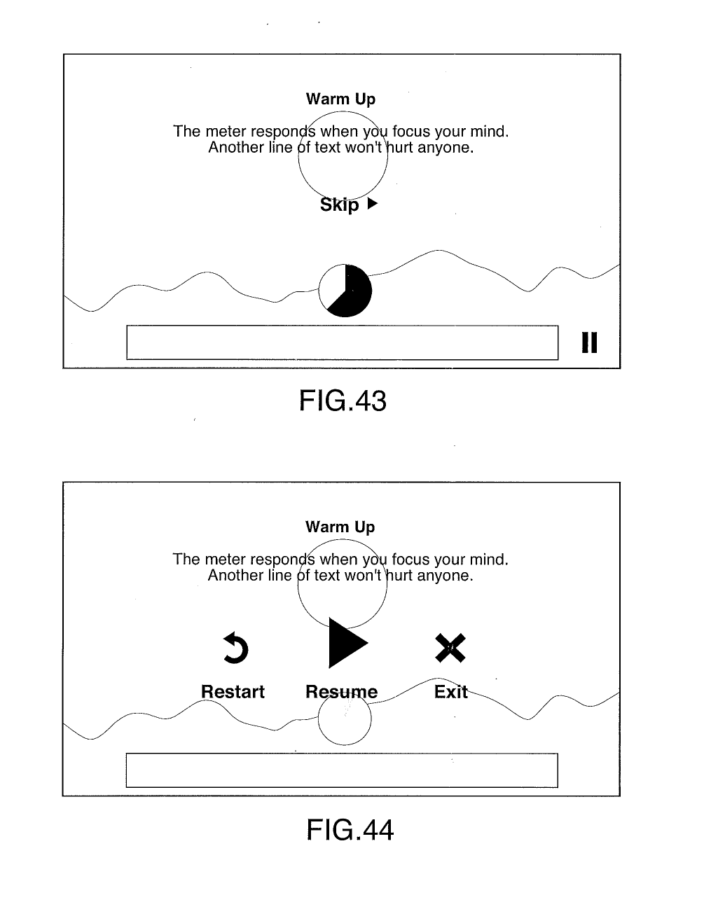

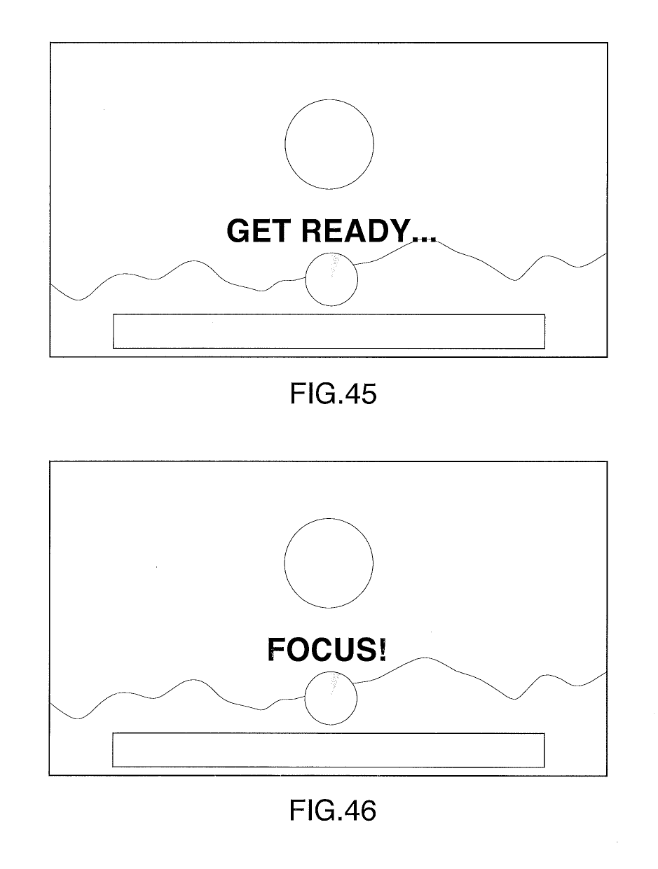

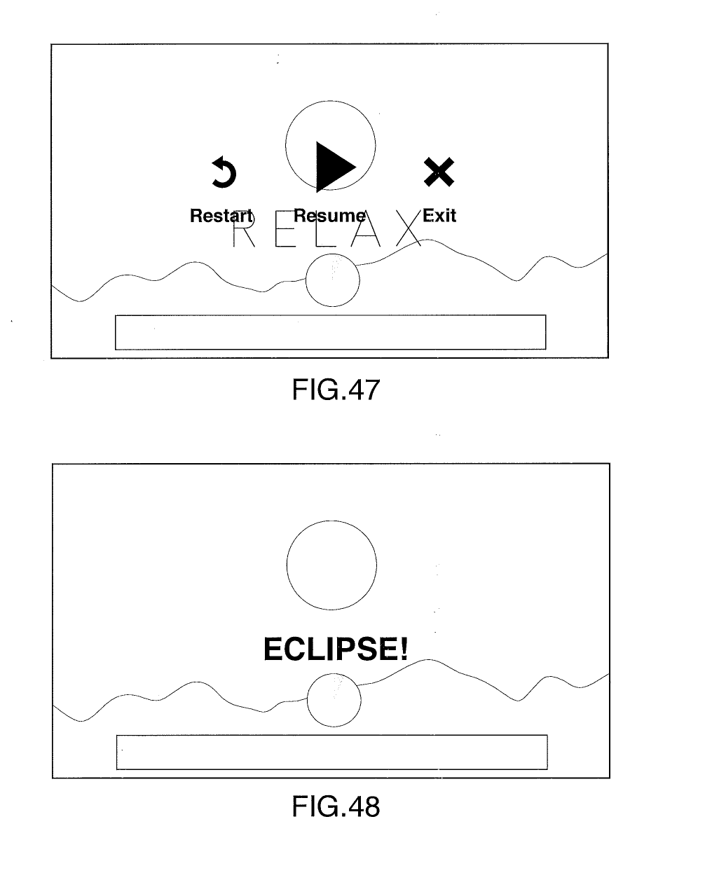

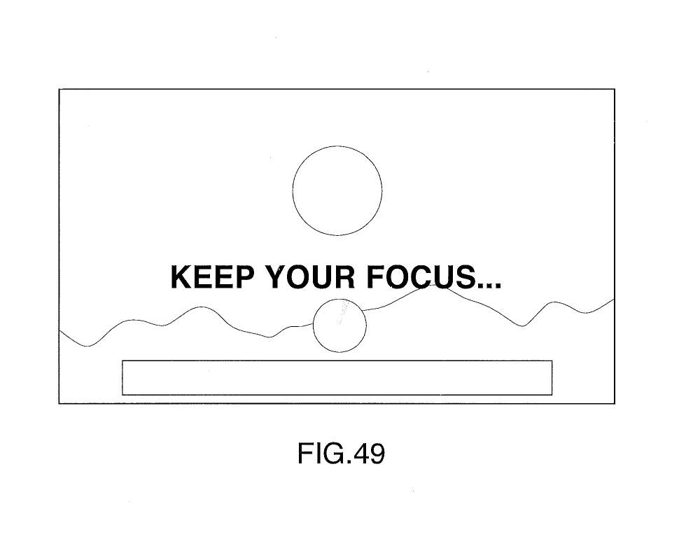

Various embodiments of the present invention are possible. FIGS. 21-49 illustrate possible embodiments of different aspects of a user interface displayed by the computing device of the system of the present invention upon executing a brain state guidance exercise. In one aspect, a user enters the computer program which may be a mobile application loaded to any manner of mobile device such as a smart phone. The mobile application may permit the user to browse a GUI “landscape” that displays one or more different exercises in a pleasing way. Optionally, the system accesses an in the cloud profile and adapts the content including landscape and suggested exercises.

The user interacts with the landscape and selects one or more exercises which may be games such as an eclipse game (based on beta-wave/focus levels), as shown in FIGS. 21-49. In one particular implementation the user may be presented with two types of feedback; first, a game context of eclipse the sun and moon when the user is a concentrating state of mind that enables them to pull the planetary objects together over time. Second, auditory feedback may be provided that enables the user to play two different musical instruments that relate to different states of mind. For example a first musical instrument may indicate an active mind, and a second musical instrument may indicate a settled mind. The computer system may switch from the sound associate with one to the other based on interpretation of brain state, or these may coincide. The preferences set by the user may include their preferred musical experiences or musical instruments. The computer program may include auditory messages to explain the exercises and their implications.

In one aspect, the computer system calculates a score based on completion of an exercise or game, and this is displayed to a user, including as shown in FIG. 21.

As shown in FIGS. 24-27 one possible implementation includes the depiction of a tree that shows circles that may correspond to progression of stages or milestones, which may include any of the following stage types: build; restore; release; relax; and refocus.

The brain state guidance of build stage type may include lessons that may challenge the user with longer and more challenging exercises, leading the user through a journey to build capacity for attention, visualization, and reflection. The build stage type exercise may test the user's ability for relaxation, reflection, awareness, and attention. Regular practice of attention exercises may be associated with increased thickness in cortical regions related to hearing, seeing and sensation. Further, regular attention exercises may slow age-related thinning of the frontal cortex. Examples of build stage type exercises may include: cultivating the user's ability to relax through focusing attention on sensations of the user's body weight, position, and movement; training the user's ability to simultaneously generate calm and gather attention through a series of exercises; building the user's ability to be mindful through exercises that train attention; developing the user's concentration through examining the contents of the user's mind, including the user's thoughts and the space between them; and drawing on the user's new skills to attempt generate a state of pure awareness.

The brain state guidance of restore stage type may include using tools the user has learned to sharpen the user's cognitive abilities, especially the user's ability to pay attention. Examples of restore stage type exercises may include: taking the user through a short exercise to direct the user's thoughts toward being aware of the user's body in the present moment; shifting the user's attention to the nuances of the user's breathing; showing the user how to push awareness of the user's body aside and immerse the user in the sounds around the user; building self-awareness by moving the user's attention through different regions of the user's body and incorporating newly learned techniques for attention and reflection; integrating what the user has learned so far, guiding the user through the awareness of the user's body and breath, through to the user's thoughts and the sounds of the user's environment.

The brain state guidance of release stage type may include assisting in relieving the anxiety, negative thoughts, and stress that that impact the health of your brain. It may do this by helping you develop a deep understanding and awareness of your breath. Focused breathing sessions may reduce negative reactions and emotional volatility in subjects presented with negative visual stimuli, such as troubling pictures. Individuals who practice focused breathing may have better control over their emotional regulation and can more adaptively respond to negative events. Users practicing focused breathing may more easily regulate their spontaneous thoughts after being interrupted by a word task, than other users. Reduced duration of the neural response may be linked to conceptual processing in regions of the brain hypothesized to moderate the stream of thought. Examples of release stage type exercises may include: bringing the user into touch with the natural rise and fall of the user's breath, in order to prepare the user for the exercises to come; bringing the user's mind into the present using the awareness of the user's breathing as an anchor; connecting what the user has learned through mindful breathing with user attention, leading the user's mind from a consideration of the user's mind, body, and breath; practicing full meditation bringing different aspects and nuances of the user's breathing into focus; integrating everything the user's has learned about mindful breathing in release stage type exercises.

The brain state guidance of relax stage type may include taking the user through a series of exercises designed to relieve tension by deepening the connection between the user's brain and body, in order to help the user to develop emotional self-awareness. Relax stage type exercises may build on one another and guide the user's attention to areas of the body affected by stress, tension, and anxiety. Examples of relax stage type exercises may include: helping the user to stimulate relaxation and get used to guiding the users' attention as emotions may show up in our bodies before our minds, such that when the user familiarizes himself or herself with the user's physical body, the user can notice emotional states when they appear and learn to be responsive, rather than reactive to them; preparing the user's body and mind for the exercises to come by guiding the user through a short relaxation practice; helping the user to develop the practice of simply noticing the sensations and activities in both the user's mind and body, building on the user's ability to become aware of their activities; extending the user's ability to relax by taking the user through a short body tension scan, as well as an examination of the sensations in the user's body; and helping to build self-awareness by moving the user's attention though different regions of the user's body.

The brain state guidance of refocus stage type may include expanding the user's awareness to include the environment, both internal and external. These exercises may help with integrating the user's awareness of the user's mind, breath, and body with the world. Examples of refocus stage type exercises include: bringing the user's thoughts, feelings, and sensations in line with awareness of the user's breathing; opening the user's mind to the sounds around the user, connecting the user to the space around the user and the present moment; beginning with attention on the body and breath, then introducing a way of reflecting on the user's own cognition; combining the lessons the user has learned in refocus stage type and show the user how to step back from the user's thoughts and make room for contemplation; and introducing the user to build insight into the user's thoughts and situate them alongside the user's body, breath, and environment.

In one aspect, the meditation exercise may include a 3-5 minute guided practice/meditation based on visual and auditory prompts from the mobile application.

The computer system may track the user's interaction with the practice/meditation and based on this the analyzer may calculate results based on the user's brain data. This may be displayed for example in a graph or results screen that may provide the user feedback or insight on a number of matters relevant to meditation related objectives. For example, the graph may include information that indicates whether the user did well or badly and at what points and this may be brought to the next exercise to improve the results.

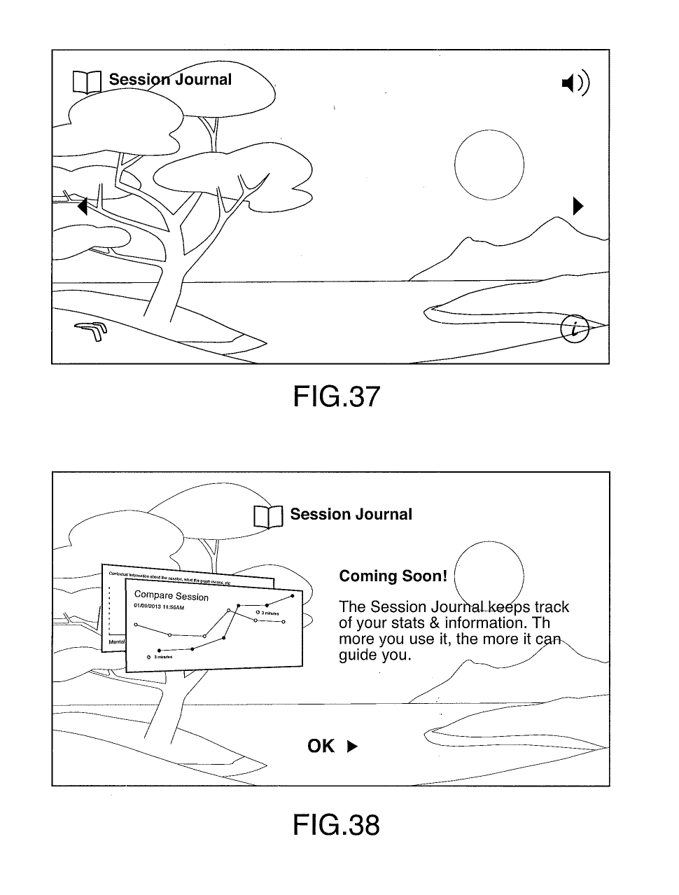

As shown in FIGS. 37-39, the computer program may include a journal that allows the user to record insights regarding why meditation related objective were or were not met.

The scores may be displayed in real time or at the end of the experience in the form of graphs, raw data, interpretive visualizations (a tree that blossoms or wilts.) Feedback can be positive (rings a bell when you're in the right state) or negative (you hear thunder when your mind wanders). Various content may be used to convey messages related to the user's performance.

In one aspect, the computer system may present training programs targeted at real-life cognitive and emotional skills that a person can use in daily life. For instance the computer program may include on more exercises designed to help a user deal with emotionally charged situations in the workplace, to manage anxiety in stressful situations, or to achieve a state of openness for creative exploration.

In one aspect of the invention, the mobile application may be configured to recommend training programs for the user to complete based on past performance, and other factors. In one implementation the computer program may include a recommendation engine that based on variety of factors generates one or more suggested training programs, which may be organized in a manner similar to a play list, that allows the user to either choose the recommended training, or optionally skip ahead in the play list, or choose something on their own.

In one possible implementation, the computer program scores the user for performance in a variety of ways. In one aspect, the user receives for example points for different positive actions of the user in relation to one or more exercises.

These points in one aspect become inputs into the recommendation engine, along with other information, such as “mood” reported by the user, energy level and other information, goals the user has selected for themselves, input from a teacher, doctor, parent, physical activity data and other data from other sensors.

During the meditation exercises, the screen may turn off when the user is instructed to close their eyes, after which auditory instructions may follow.

The computer device may include a mobile device that includes the mobile application, but the mobile application may in part connect to another computer device that includes a larger screen that is used to provide a more immersive visual experience.

The computer system may be link to a web portal to for example download additional exercises and access a dashboard that helps users past experiences and performance relative to meditation related objectives.

The computer system may communicate with and the computer program may integrate information from other sensors measuring bio and other data (heart-rate monitor, blood pressure monitor, blood glucose monitor, phone accelerometer, microphone etc.) in order to obtain better overall information regarding the user.

In another aspect, the computer program may enable the user to access a “teacher console” which could be part of the computer program or a separate computer system, resource or computer program, that enables a teach or instructor to view data of their students, review their practices and assign lessons or exercises individually based on the person's unique needs and interests. Meetings such as videoconferencing sessions may be organized to coach a student including using features of the computer program. For example the system may be used to interact with a mindfulness coach via the Internet.

The console view could also be used by doctors to monitor compliance with a prescribed training regimen, or by a concerned parent working with their child to address behavioural/emotional problems.

System interactions may also be initiated on a peer-to-peer basis. For example a user may share their information with a friend who then can log into the user's information. Results may be also be shared through various external sites such as a social networking platform.

Brainwave information can be shared between computer devices for a number of reasons. For example in one implementation users may receive as information from the computer system brainwave information for another user across the Internet. This information may be made available in real time or delayed for example such that two friends in a social network can perform the same practice at different times and hear in conjunction with the practice how their friend did at various time for example as means of encouragement or motivation. A user may also try to achieve scores based on a profile of somebody that they do not know who may for example be a special user such as a well-known expert in meditation, or a profile based on normalized score based on demographic traits (such as average North American male).

In one aspect, the computer system may enable one or two-way interaction including for example for organizing group meditation sessions. These may be led for example by a meditation expert for significant user groups leveraging computer networks. In one implementation, an Internet application may be linked to the computer system and enable one or more meditation leaders or teachers to utilize a live stream to guide a meditation session of a large number of users, where the Internet application provides a console view to the leader or teacher to provide access to data for the users, either individually or in aggregate. So for instance, he might choose a particular user to view (“squishymama42 you are doing great!”) or look at groups of users by demographic group or geographic region (“Ladies, you are destroying the men right now! Way to go!” or “India, I see you coming up beautifully! Oh here comes Europe!”) or any other way of selecting people (Premium members: “And my special premium club members are doing extra well today!” New members: “These are the best scores I've seen from any group of newcomers!” People who have indicated they have trouble sitting for long periods of time. “Hang in there! I see you wavering. We'll be done in a few minutes!”)

The computer system may be linked to other types of sensors and applications. For instance, a user can measure stability of mind while running—and these scores could feed into, or take data from the Nike iRun app/accelerometer. This enables a user who is jogging to discover the correlation between mental state and steps per minute, speed or calories burned.

The computer system may use time and location data from GPS to understand the user's location and based on this recommend exercises for that location. i.e. on your morning commute to work it may recommend a “get up and go” training, at home at night it recommends the “unwind and relax” program.

Users may create and upload their own meditation practices for others in the community to download and try. Users could set their own parameters for what state is desirable, and the computer program may score based on that input. An aggregate score for the entire population of users may be calculated and scored in a visible manner.

The computer system may interface with external peripherals that provide additional information, feedback or provide ambience. These could be, for instance, a BLUETOOTH™ meditation bell that rings at the beginning and end of a session, or a light that changes the ambient brightness and colour of light based on a user's brain state.

### Motivating the User to Brain Train

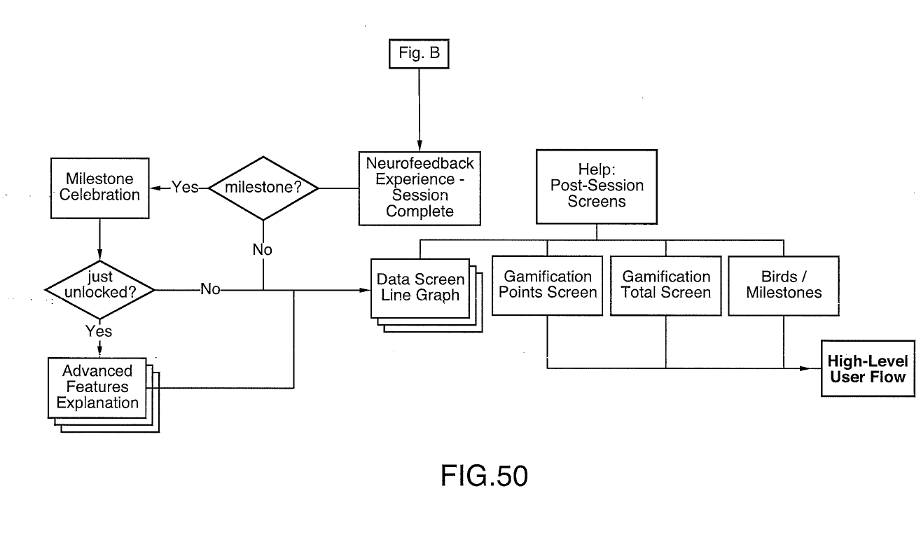

One of the obstacles to having a user self-administer ABCN training surrounds motivation. As the benefits are gradual and require consistent training, the system must be designed to encourage users to adhere to the program. FIG. 50 shows a user flow of feedback given to a user about their ABCN session(s).

There is a variety in the types of feedback that may be employed by the present system. For example, multiple audio/visual environments within which to practice can help users experience more variety despite self-administering a fairly repetitive ABCN exercise.

User engagement with the system may also be increased by, directly after session, showing users their progress through the session through a series of graph modes fully engages users with their performance, as they remember the session in different ways. Having different data views allows different users with different experiences find data which connects with their experience. Examples of data views which may be provided by the system include: time vs. score line graph; percentage of time in target/other states pie chart; bar graph comparing chunks of time (beg, mid, end) across one session; and brain topography which allows users to see a colour-coordinated a spatial map of their head as triangulated by electrodes of the brain sensor. Long-term data modes may also be provided by the system to allow users to see their session history in interesting ways. Examples of these modes may include: scrollable month-by-month views which show individual sessions on an absolute scale (showing where users' calibration was, and where their performance during the session fit in context of that calibration); Insights screen which provides information relevant to the user's life which can be gleaned from analysis of usage/performance data; and calendar-style view which enables users to view the following information by month or week (e.g. number of sessions; total performance; average performance; and time spent practicing).

The system may be configured to provide very simply graphing modes as overly complex screens may not be appropriate. Users may be less interested in a powerful data dashboard, and more interested in clear insights which tie back to their own experiences during the session.

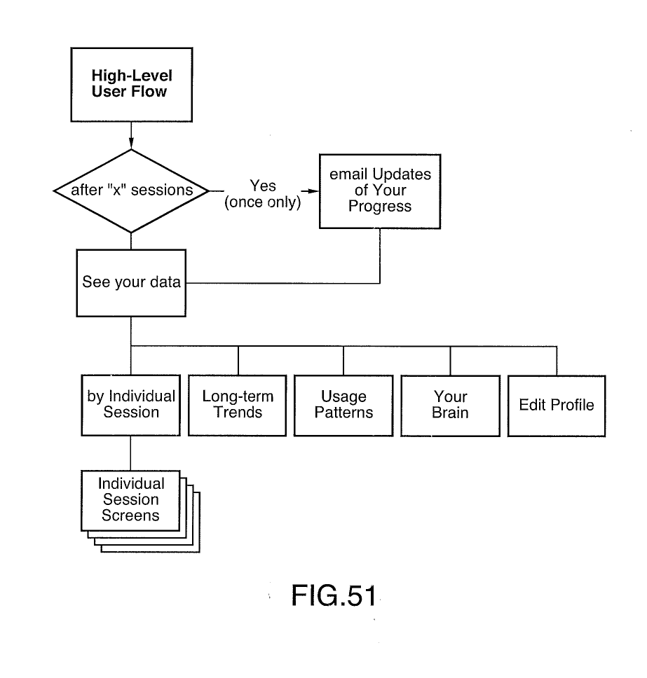

FIG. 51 shows a data visualization user flow of screens that can be selected to view the user's results and progress.

Interactive video games are notorious for their ability to drive motivation. Recent trends in “gamification” demonstrate how many systems and methods can benefit from elements of game design. Adaptive Brainstate Change Notification is certainly no exception. However, due to its therapeutic value as a treatment intervention, it may not be appropriate to rely too heavily on arbitrary, artificial extrinsic motivators, as they tend to be temporary when compared to intrinsic motivation. For this reason the system of the present invention may incorporate different types and levels of gamification and to apply a suitable balance and type of gamification for self-administered ABCN training.

In one example, the system may gamify ABCN by to providing points for time spent in the target state. However, this approach can lead to self-judgement and a negative user experience, as users often don't score very high as the target state of a ABCN exercise can be difficult to reach. This problem may be solved by the present invention by evolving the paradigm to provide many points for seconds spent in the target state, and lower gradations of points for approaching the target state (i.e. users are still awarded points for being close to the target state, but not as many). This way, most users score points, but users' point totals get exponentially larger as they get close to and enter the target state. This attempts to ensure that all users have a good experience, and those who perform better still feel adequately rewarded.

In another example, emergent audio properties are counted and scored on a separate scale as “bonus” points. This is a playful secondary point system and allows the user to quantify a different attribute of their session: consistency. These “bonus” points are awarded for long streaks of consistent/stable presence in the target state. This is a further reward for those users who perform exceptionally well. For users who don't do as well, they still earn points but fewer “bonus” points. In this way, moderate performing users still feel they have been rewarded with points, but they also see there is room for better and more consistent performance.

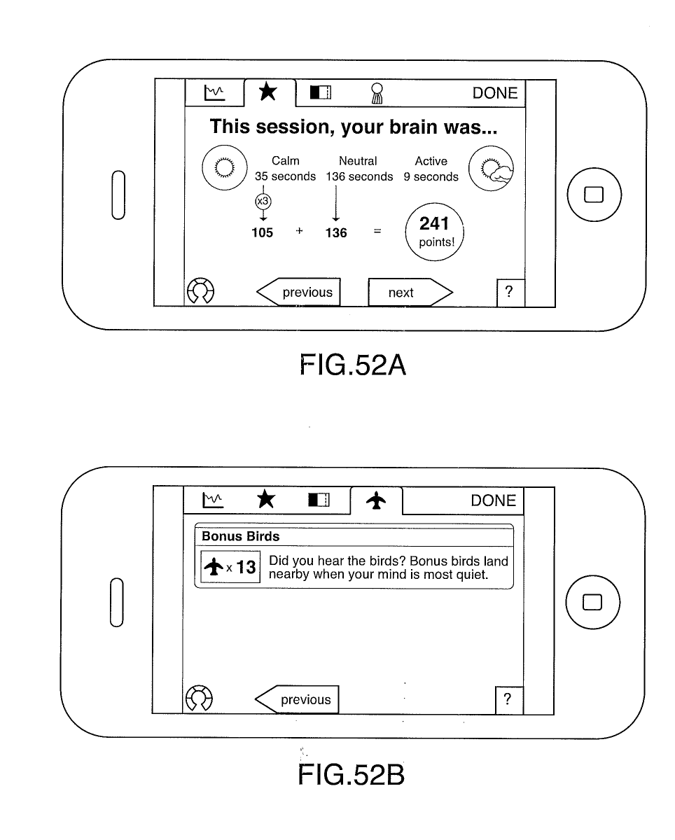

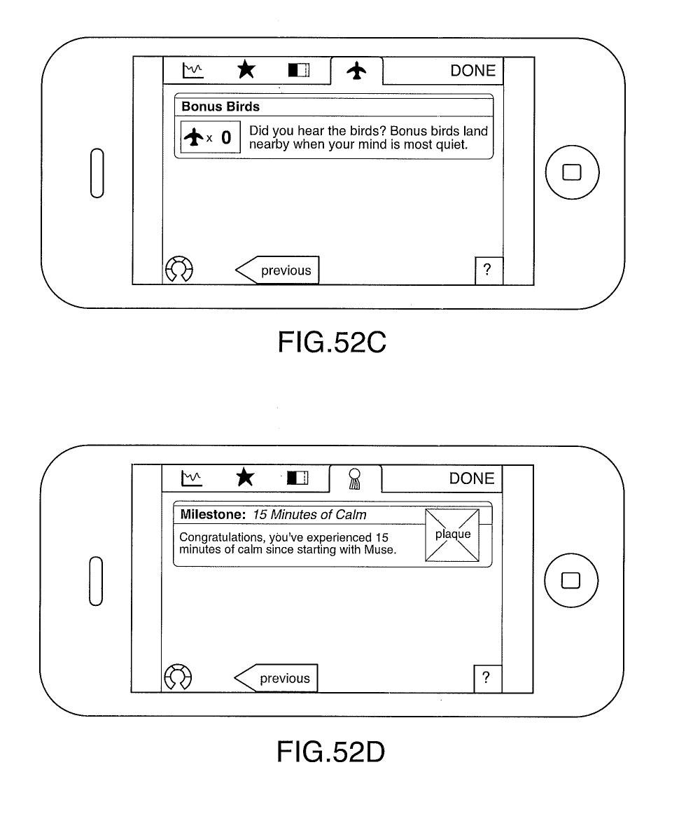

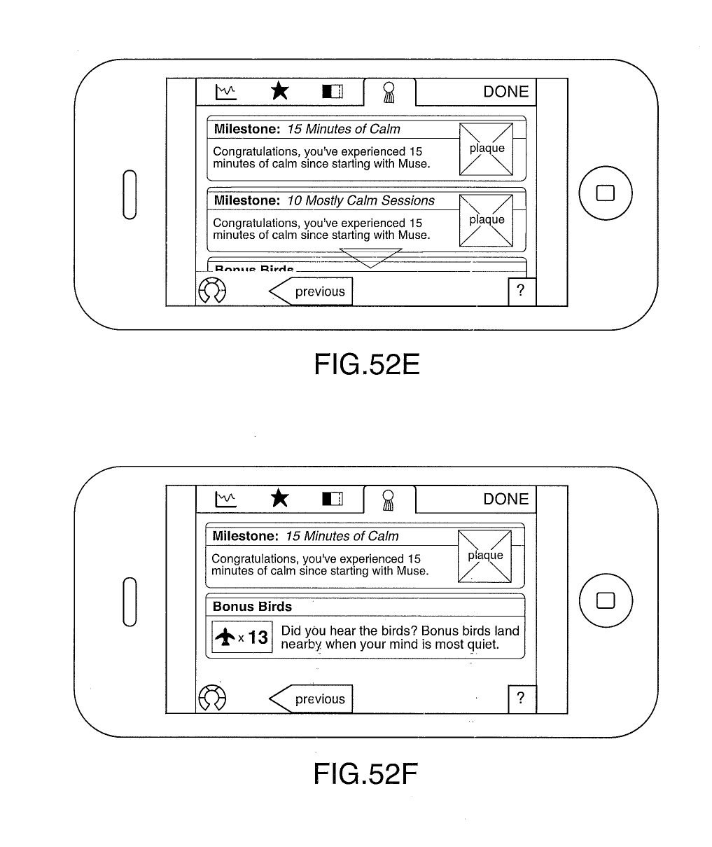

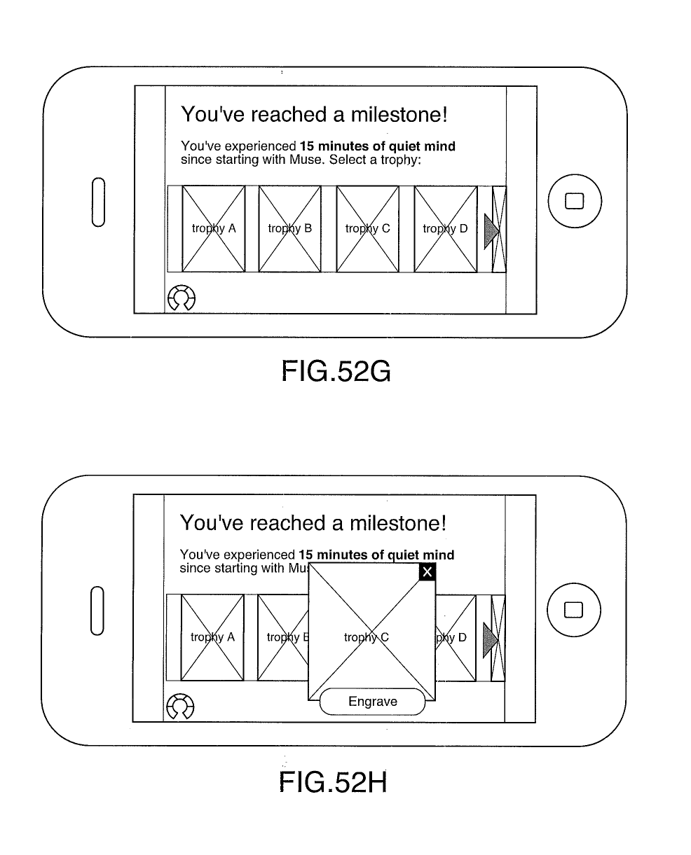

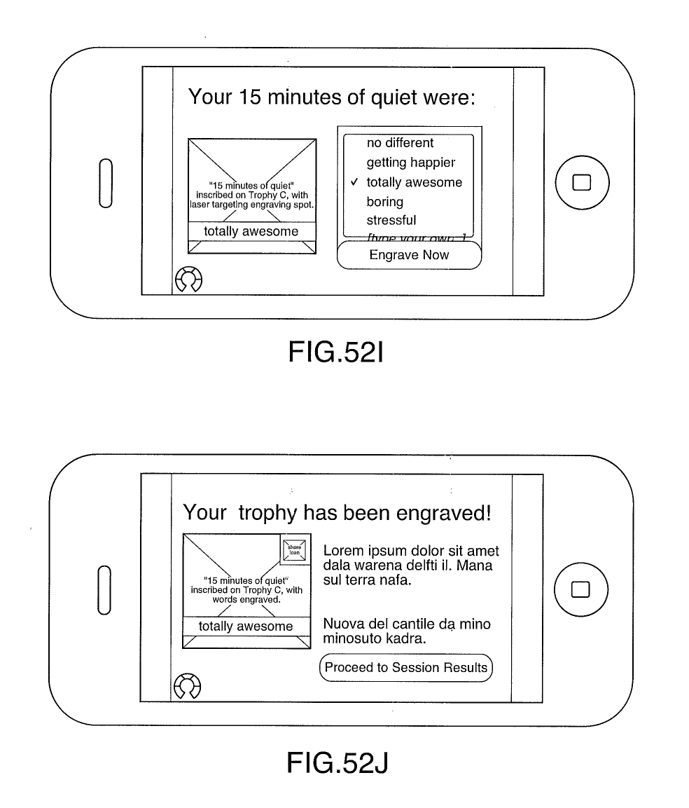

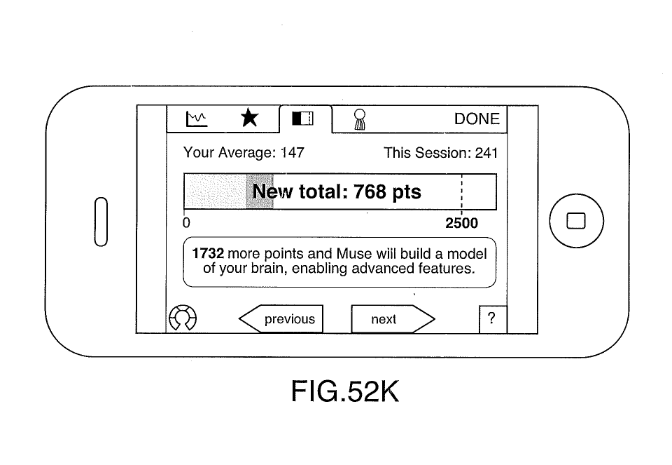

The system may employ the typical gamification technique of “badges” or “achievements” with self-administered neurofeedback in a variety of ways. For example, the playful route, with cute and game-like awards for random data patterns (i.e. if a user does a session every Monday, they get a “Monday” achievement). In another example, there is provided a more serious approach which simply quantifies the user's long-term data and rewards great achievements. These achievements may be called “milestones” to match their seriousness. A number of milestones may be incorporated into the system design of the present invention, including: total all-time number of seconds spent in target state; total all-time number of sessions; gradations of exceptionally performing sessions; total number of sessions where the majority of time was spent in target state; total number of consecutive daily sessions (<24 hrs apart); total number of “bonus” points; total number of session set to the maximum length of time; and total number of sessions in a single day. FIG. 52A-52K show examples of gamification screens indicating rewards earned or other gamified feedback.

In order to promote long-term engagement, long-term “unlocks”, rewards for earning many points with the system, may be employed by the system. However, it may not be desirable to lead users to treat the system as more of a game than a self-administered treatment mechanism, as such a result would represent an overuse of game design principles. Accordingly, the present invention may employ a single unlock for new users.

For example, in the first few sessions, users may accumulate points toward a first “unlock”. As opposed to the game-like idea of having that “unlock” provide access to features of the app, it is framed as the point where the system will be able to build a model of the user's brain. When users reach this goal, the system will have accumulated enough calibration data to be more confident in its ability provide responsive and accurate feedback (using a trailing average of previous session data as calibration for every session). The user will have done enough sessions to make long-term data views engaging and relevant—the system can unlock these features at this point. This not only ensures that empty long-term data screens are never seen by the user, but it also allows these screens to appear as based on the “model of the brain” users have unlocked. Users may be presented average session data based on their first run of sessions, and this data will be used to recommend a weekly personal point goal for users. In this way, the initial gamified extrinsic motivation developed in the first few uses of the app are handed off directly to intrinsic motivation. By asking users to set a personal goal, the system effectively asks them to reflect on the benefits they've experience from their self-administered ABCN training. Once they determine how much they want to use it, they are able to set their weekly personal goal. From this point on, the system uses the exact same interface element which were initially used for the “unlock” goal (extrinsic motivation), to quantify and keep track of personal goals (intrinsic motivation). In this way, this self-administered neurofeedback system engages users first with an extrinsic motivator, and seamlessly inspires users to develop intrinsic motivation for system use. In this way, the system avoid feeling like a game, resulting in a UX which feels more like a training and tracking tool. The screen presented to users directly after their session should provide clear guidance as to the purpose of that unlock. Showing users the average score, current score, and total score can help provide context. Visually showing the progress toward a goal is critical, and the labelling of that goal needs to be a clear and engaging communication of how the system will respond at the unlock point.

While most of the innovations listed here focus on the self-administration of a ABCN training paradigm, gamification elements create an environment which feels a bit more fun. This leads to users wanting to experiment and explore the application beyond pure ABCN training. In order to satisfy this user need, the system may incorporate a “challenge mode”—this a special environment where users can play with the ABCN system's responsiveness in different ways, in an attempt to meet different goals. This mode represents a more clear “game” mode, where users have to accomplish certain goals to proceed through a linear series of “levels”. Some examples of goals may include: achieving the opposite end of the spectrum for a certain period of time; achieving a certain level of volatility; achieving a certain amount of continuous “bonus” points; and achieving a certain overall score.

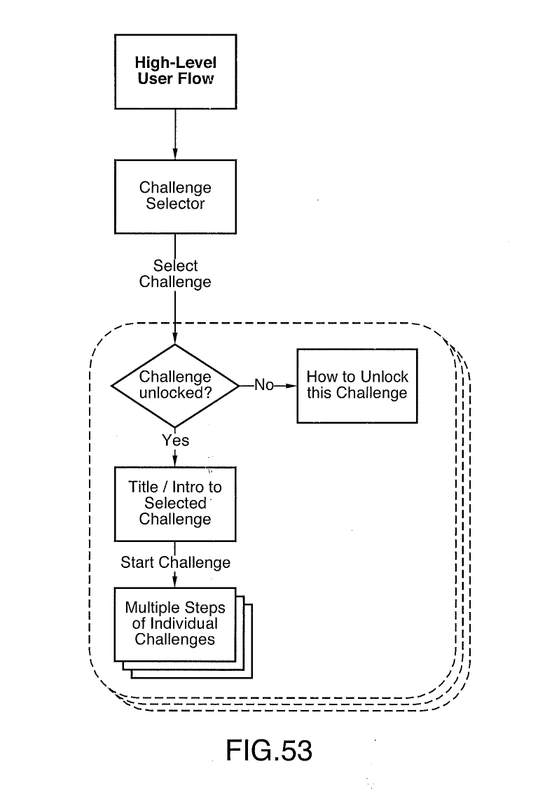

FIG. 53 shows a Challenge Mode User Flow that describes the flow if a user selects to have more challenging exercises that need to be unlocked or different goals.

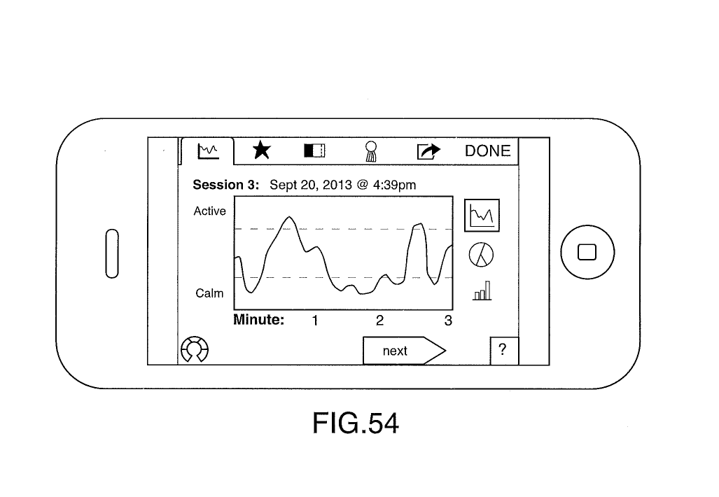

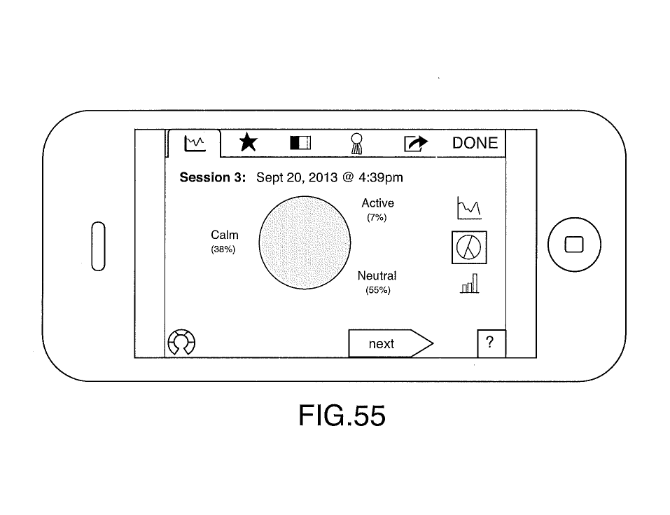

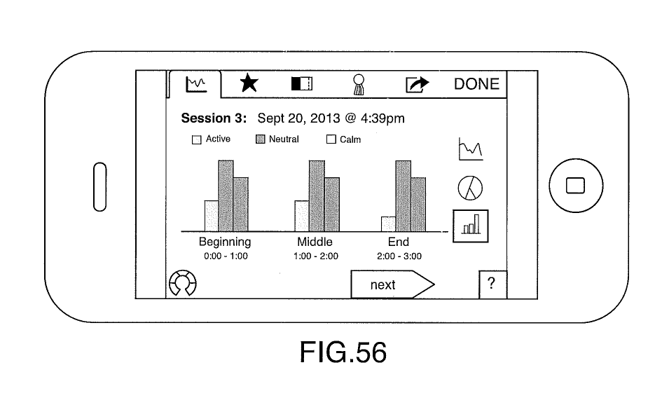

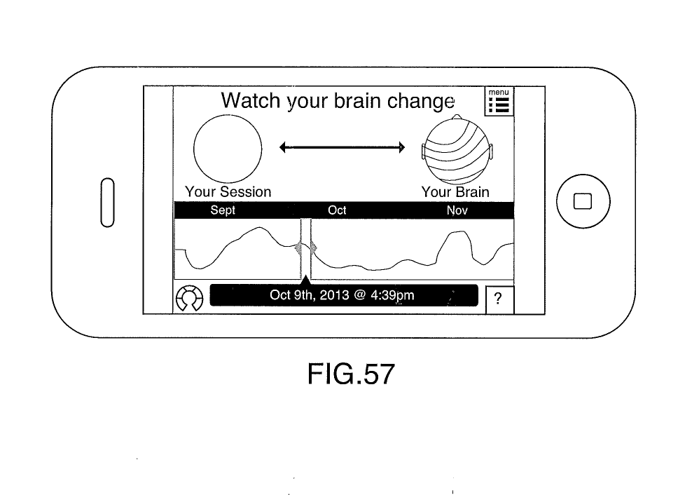

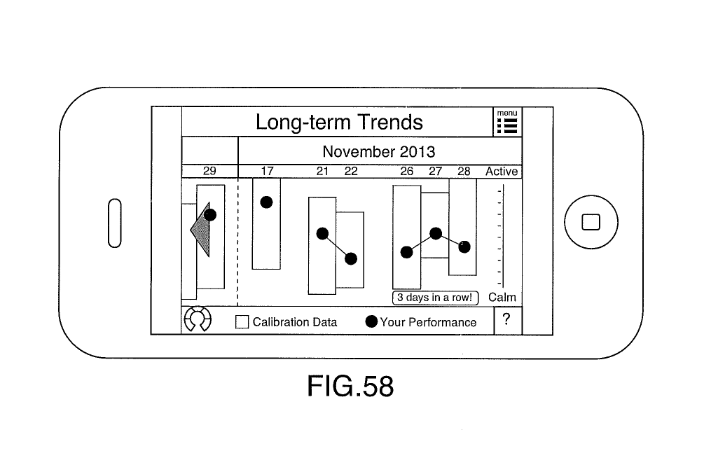

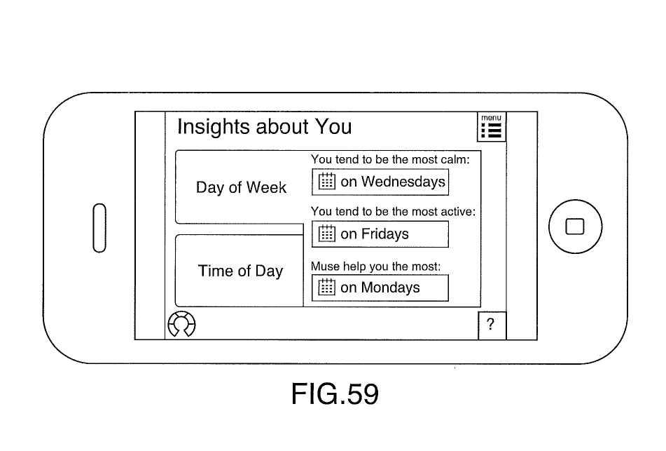

Various feedback screens may also be generated by the system to help to motivate the user to train. For example, FIG. 54 shows a time vs. score line graph type of feedback. FIG. 55 shows percentage of time in target/other states pie chart. FIG. 56 shows a bar graph comparing chunks of time (beg, mid, end) across one session. FIG. 57 shows a brain topography which allows users to see a colour-coordinated a spatial map of their head as triangulated by electrodes of the brain sensor. FIG. 58 shows a long-term data mode which allow users to see their session history in interesting ways, scrollable month-by-month views. FIG. 59 shows insights screen which provides information relevant to the user's life which can be gleaned from analysis of usage/performance data. This may be a calendar-style view which enables users to view the following information by month or week: number of sessions; total performance; average performance; time spent practicing.

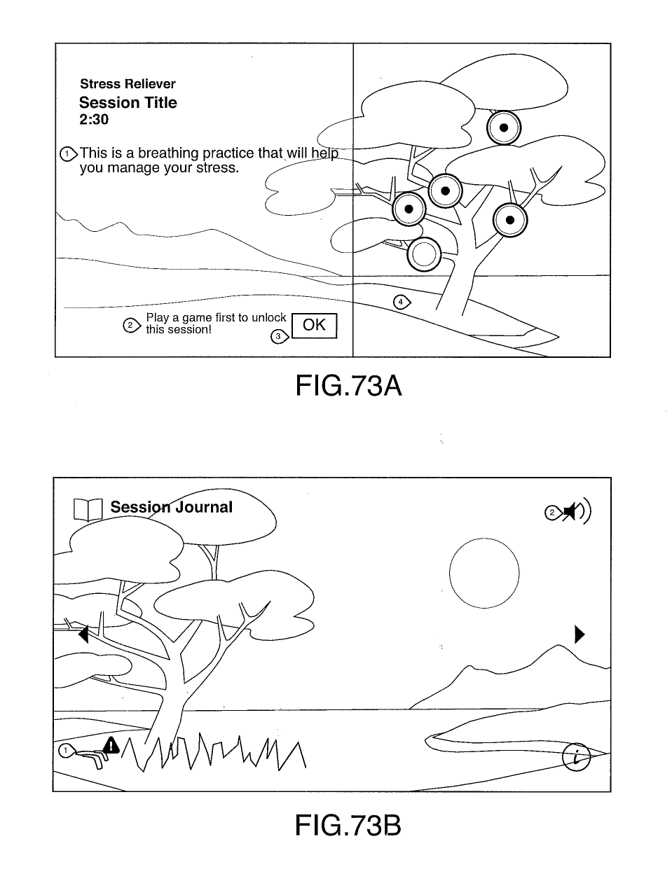

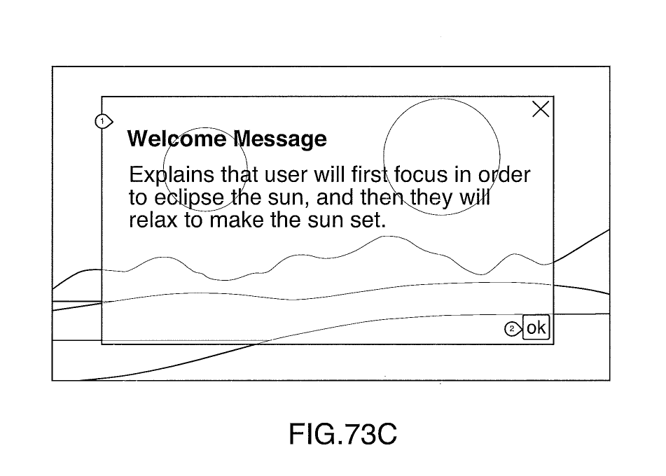

In an example of a brain state guidance exercise of the system of the present invention, the user may be presented with a landscape metaphor for brain state guidance. The user may select to display or hide HUD elements. A visual prompt may appear to start the exercise, optionally by panning the image to one side of the display of the device. Different modes may be available for the exercise. Examples of a “Career” mode, a “Home” mode or home screen, and an “Arcade” mode may be shown in FIGS. 73A-73C.

### Evidence-Based Brain Training Methods

Brainstate training may be defined as the act of maintaining a particular state by applying oneself in an exercise that engages you to continually monitor when you are sustaining or disengaging from the desired state and to then re-apply oneself back to the chosen state. This repetitive action creates the environment for operant conditioning to take effect allowing the person who is training to enter the desired state with enhanced volition.

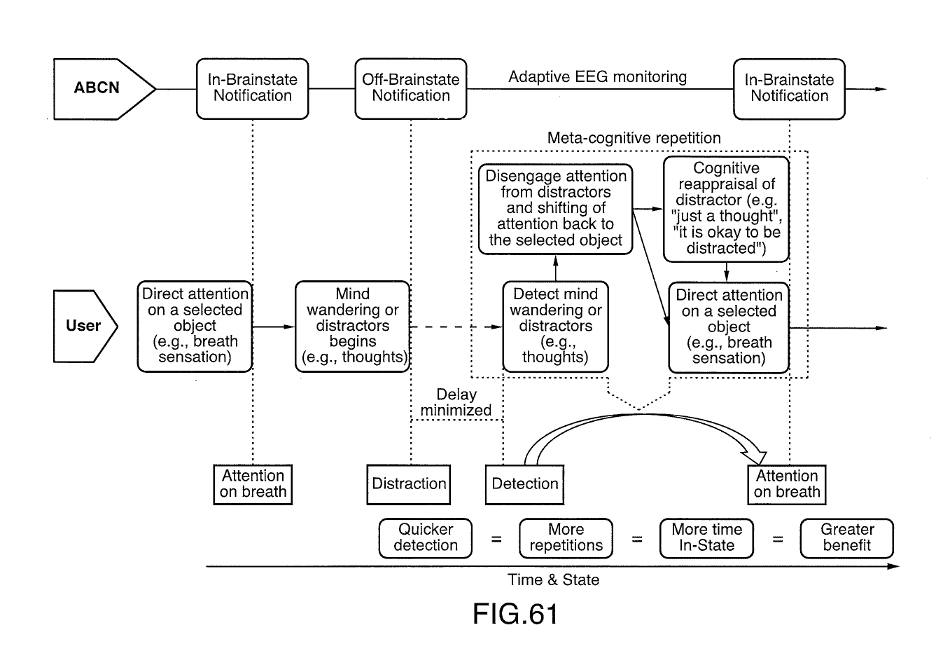

ABCN may be used to train the brain. Very much like a muscle can be built through repetitions of an activity, so can the brain. The way we do this is through “meta-cognitive” repetitions—noticing when you have disengaged from your selected brainstate and then re-applying yourself back to the exercise. ABCN helps the trainee to engage in more of these meta-cognitive repetitions than they would have if left unsupported, yielding decreased time delay in noticing disengagement from exercise and increased time in selected brainstate (see FIG. 61).

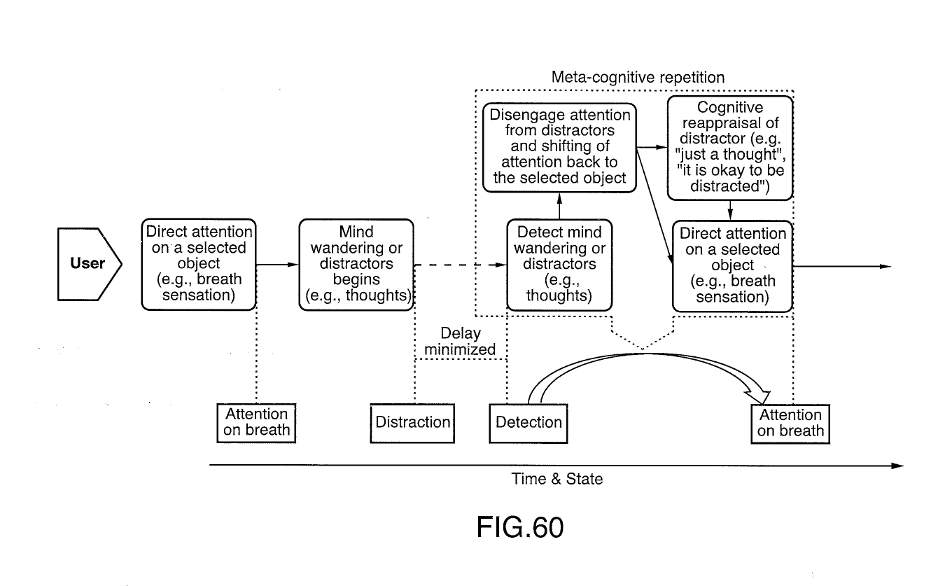

In brainstate training, the natural tendency of an untrained mind is to disengage from the exercise/brainstate. An unsupported user is prone to delays in course correcting back to the exercise/brainstate, the length of delay varying on their skill level (see FIG. 60). A supported user receives notification when they have disengaged from the exercise/brainstate and the delay is minimized (see FIG. 61).

In accordance with a general aspect of the present invention, electroencephalography (EEG) based biofeedback is combined with mindfulness-based practice (MBP) to improve brain health. There are a number of different brain training methods. These include: MBP, Focussed Attention Training, Open Monitoring, Compassion Based Practices, and Visualization. Its intention is to establish an optimal state of being through methods that support familiarization and cultivation of physical, cognitive and emotional well-being. A participant comes to it through primary intentions: self-regulation, self-exploration and self-liberation. These intentions are dynamic in nature throughout a person's lifespan.

ABCN supports brain training methods by supporting the user to monitor their attention, sustaining of attention, specific instructions and display of progress. The present system may detect changes in the user's brain state and classify the new brain state and present a notification that is meaningful and relevant to the user's goal brain state. The app/system of the present invention may have built-in scripts that embody the instructions that has a voice actor to provide the instructions for the user to complete the exercise. The system may also provide visual data graphical display of users progress over time.

The benefits of these methods are supported by evidence from the literature identified by reference numbers at the end of the description. Health outcomes such as reduced rumination [1], stress reduction [2], boosts to working memory [3], increased focus [4], less emotional reactivity [5], greater cognitive flexibility [6] and improved relationship satisfaction [7] have been observed. Neuroscience research has shown that these brain training methods can change the default behaviours of the brain, through EEG and fMRI studies [8, 9]. Other fMRI studies point to structural changes in the brain associated with brain training methods such as increased axonal density and myelin thickness [10-12].

### Brain Training Method: Focused Attention

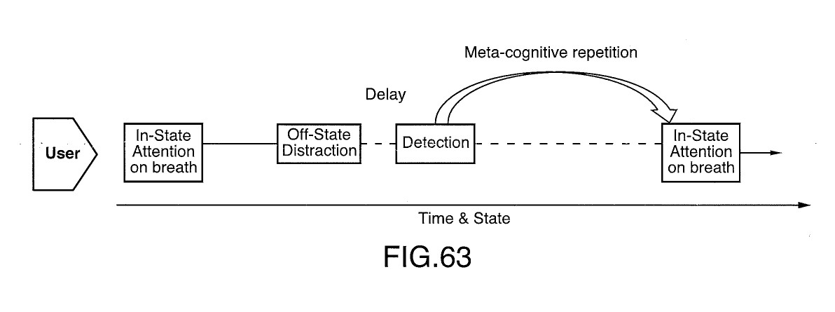

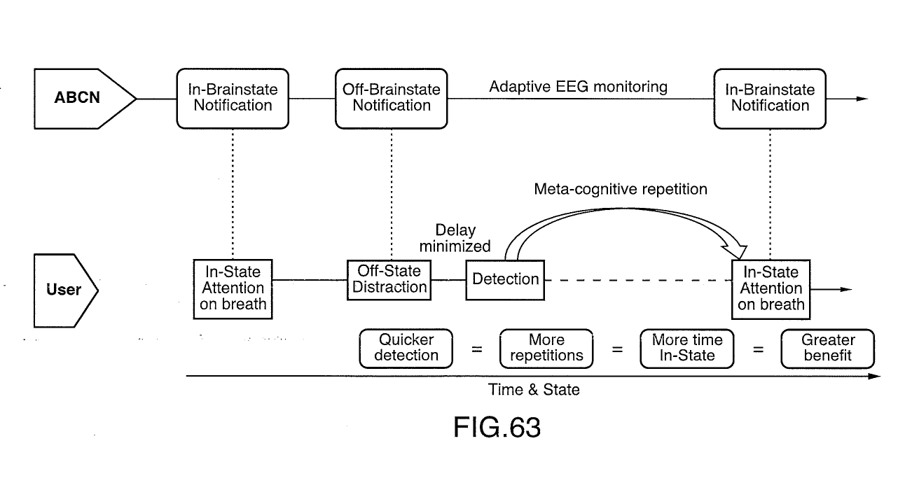

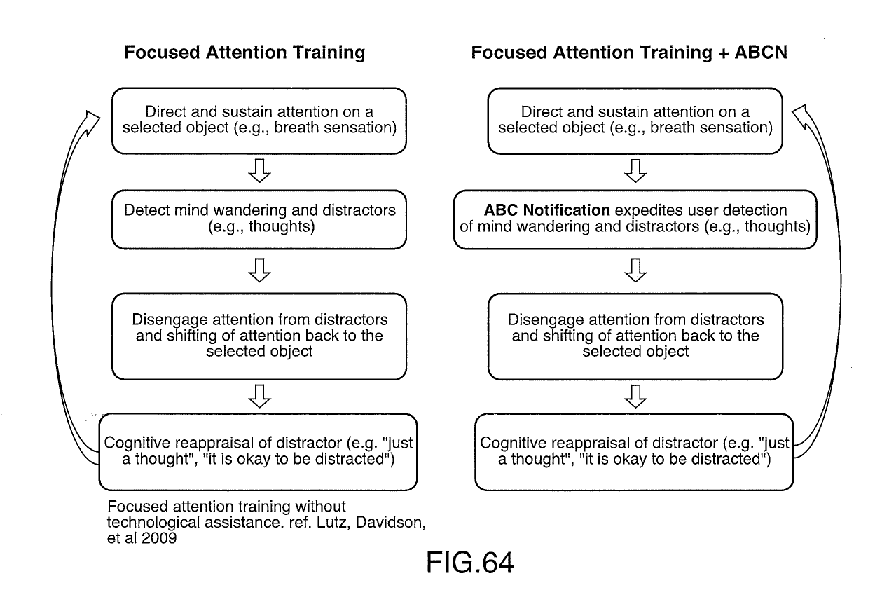

In a Focused Attention Method of brain training, the user sets the intention to sustain attention upon an object with the goal to cultivate relaxation, stability and vividness of attention. Attention can be placed upon sense objects (physical, visual, auditory) and mental objects (visualization, thoughts). An ABCN “in-state” may include apprehending object of attention, sustained attention, and alert yet relaxed focus. An ABCN “off-state” may include drifting into thinking, mind wandering, and anxiety or drowsiness. FIG. 62 shows a Focused Attention Method of training, without ABCN, and FIG. 63 shows a Focused Attention Method of training with ABCN. Further overviews of the Focused Attention Method with and without ABCN is shown in FIG. 64.

An example of a focused attention exercise script where attention is focused upon the breath, without ABCN, may include: posture in a supine or seated position; breathing in a natural rhythm; Attention on the tactile field of the body (phase one—relaxation); Attention on the rise and fall of the abdomen (phase two—stability of attention); Attention on apertures of the nostrils (phase three—cultivating vividness of attention); Count your breaths; and Intention to Enhance present moment attention and working memory with introspection.

An example of a focused attention exercise which may be employed by the present system, with ABCN, may include: directing the user to, when ready, sit in a comfortable position and close eyes, allowing user's back to be straight with relaxed shoulders; directing the user to settle in, take a few moments to relax by becoming aware that you are breathing; to help focus on breathing, directing the user to count each out-breath up to 10, then start back at 1, and begin the count again; directing the user to start back at 1 and resume the count without judgment when noticing that the mind was wandered or count has been lost; prompting the user to begin, and playing a starting sound, starting a timer, and starting ABCN control of wind.

Focused Attention training may include: directing and sustaining attention on a selected object (e.g., breath sensation); detecting mind wandering and distractors (e.g., thoughts); disengagement of attention from distractors and shifting of attention back to the selected object; and cognitive reappraisal of distractor (e.g. “just a thought”, “it is okay to be distracted”).

A major component of Brain Training Methods may be Focussed Attention Exercises (FAE) which is an arduous process that involves many hours of blind training, where the practitioner receives little to no feedback or guidance. FAE has strong evidence supporting its health benefits yet it remains inaccessible to many individuals. Focussed attention is the ability to concentrate at a task at hand and not be distracted. Biofeedback using EEG (known as neurofeedback) has been shown to be effective therapy for Attention Deficit Hyperactivity Disorder, pain, substance-use, depression and sleep [13, 14]. Because of the success of neurofeedback for these disorders, we believe that ABCN may be useful for improving effectiveness of FAE. In order to make FAE more accessible the system may develop a score indicative of “stability of attention” during FAE while users wear a portable and consumer-friendly headset recording their EEG signals. Machine learning of brain signal features will be used to derive an objective score to provide real-time feedback to the user on the efficacy of their practice, in hopes of enabling practices on-the-go while guiding and motivating further practices.

Focused attention may be its own distinct form of training, and may be utilized within mindfulness-based brain training method along with open monitoring. A style of MBP consists in sustaining selective attention moment by moment on a chosen object, such as a subset of localized sensations caused by respiration. To sustain this focus, the meditator must also constantly monitor the quality of attention. At first, the attention wanders away from the chosen object, and the typical instruction is to recognize the wandering and then restore attention to the chosen object. For example, while intending to focus on localized sensations around the nostril caused by breathing, one may notice that the focus has shifted to the pain in one's knee. One then “releases” this distraction, and returns to the intended object. Thus, while cultivating the acuity and stability of sustained attention on a chosen object, this practice also develops three skills regulative of attention: the first is the monitoring faculty that remains vigilant to distractions without destabilizing the intended focus. The next skill is the ability to disengage from a distracting object without further involvement. The last consists in the ability to redirect focus promptly to the chosen object.

Progress in this form of meditation is measured in part by the degree of effort required to sustain the intended focus. The novice contends with more distractions, and the three regulative skills are frequently exercised. As one advances, the three regulative skills can be developed to the point that, for example, advanced practitioners have an especially acute ability to notice when the mind has wandered. Eventually FA induces a trait change whereby the attention rests more readily and stably on the chosen focus. At the most advanced levels, the regulative skills are invoked less and less frequently, and the ability to sustain focus thus becomes progressively “effortless.”

In advanced practitioners, FA practices create a sense of physical lightness or vigor, and the need for sleep is said to be reduced. Advanced levels of concentration are also thought to correlate with a significant decrease in emotional reactivity. FA practices typically involve a relatively narrow field of focus, and as a result, the ability to identify stimuli outside that field of focus may be reduced.

In an ABCN supported focused attention training method of the present invention, the system of the present invention may support the user within the various brain state guidance exercises to cultivate mindfulness, placing a non-judgmental attention upon: physical sense object; body, breath, sound, sight, etc.; feelings/emotions; thoughts; volitions.

### Brain Training Method: Open Monitoring

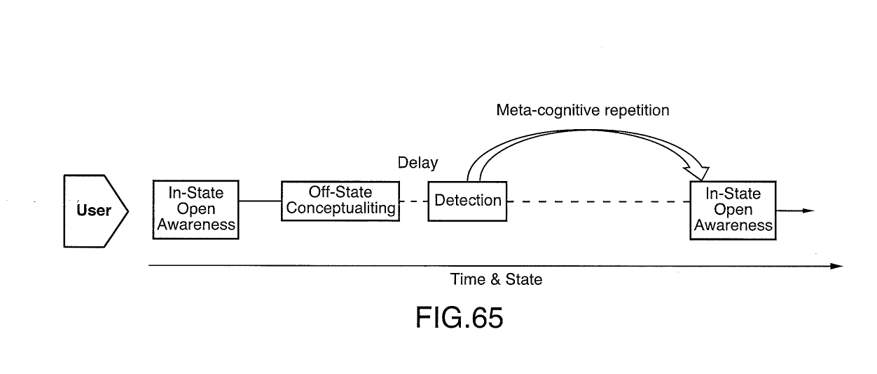

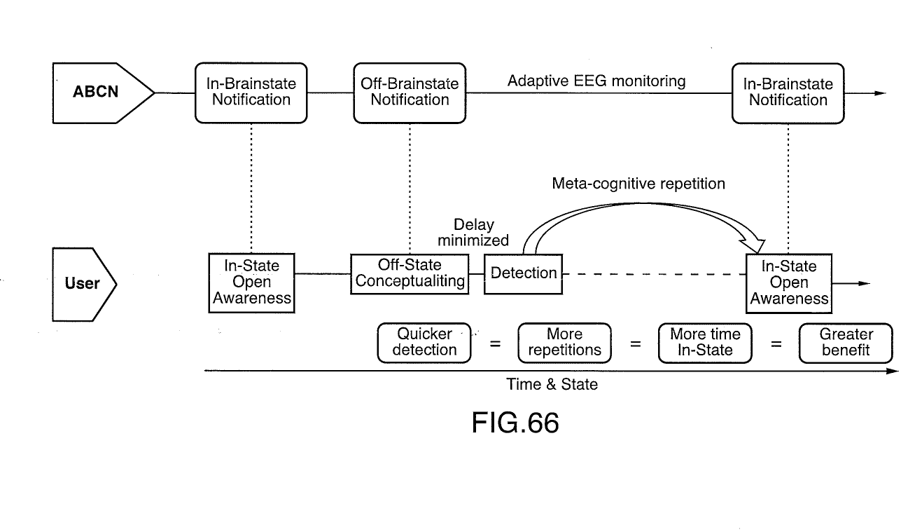

In an Open Monitoring Method of brain training, the user sets the intention to engage in open awareness and labelling of physical, emotional and mental sensations with the goal to decenter from rumination and rest within the limpid clarity of a non-conceptual state. An ABCN “in-state” may include experiential focus, unbounded observation of moment to moment experiences entering sense field. An ABCN “off-state” may include narrative focus, drifting into rumination or analysis/interpretation of experiences entering sense field. FIG. 65 shows an Open Monitoring Method of training, without ABCN, and FIG. 66 shows an Open Monitoring Method of training with ABCN.

An example of an open monitoring exercise script for settling awareness of awareness alone may include: Posture in a supine or seated position; Breathing in a natural rhythm; Rest evenly—relaxed, still, and vigilant; Release any thoughts that come to the mind; Settle awareness on awareness alone; and Intention is to decenter from rumination and rest within the limpid clarity of a non-conceptual state.

Open Monitoring training may include: no explicit focus on objects; non-reactive meta-cognitive monitoring (e.g. for novices, labeling of experience); and non-reactive awareness of automatic cognitive and emotional interpretations of sensory, perceptual and endogenous stimuli.

While varied, OM practices share a number of core features, including especially the initial use of FA training to calm the mind and reduce distractions. As FA advances, the well-developed monitoring skill becomes the main point of transition into OM practice. One aims to remain only in the monitoring state, attentive moment by moment to anything that occurs in experience without focusing on any explicit object. To reach this state, the practitioner gradually reduces the focus on an explicit object in FA, and the monitoring faculty is correspondingly emphasized. Usually there is also an increasing emphasis on cultivating a “reflexive” awareness that grants one greater access to the rich features of each experience, such as the degree of phenomenal intensity, the emotional tone, and the active cognitive schema.

Although the enhancement of the monitoring awareness continues until no explicit focus is maintained, the monitoring itself does not create any new explicit focus. Thus, unlike FA, OM involves no strong distinction between selection and de-selection. For example, the FA monitoring faculty detects a state's emotional tone as a background feature of the primary focus, but in OM the emotional tone is detected without it or any other object becoming an explicit or primary focus. It is as if emotional tone and such remain in the background, even though there is no contrasting cognitive foreground. In this way, the “effortful” selection or “grasping” of an object as primary focus is gradually replaced by the “effortless” sustaining of an awareness without explicit selection.

This distinction between the “effortful” and the “effortless” points to the contrast between skills employed during the state and traits developed as practice progresses. For example, initially the practitioner frequently “grasps” to objects in a way that requires the skill to deliberately disengage that focus, but eventually a trait emerges such that one can sustain the “non-grasping” state, which has no explicit focus.

A central aim of OM practice is to gain a clear reflexive awareness of the usually implicit features of one's mental life. It is said that awareness of such features enables one to more readily transform cognitive and emotional habits. In particular, OM practice allegedly leads one to a more acute but less emotionally reactive awareness of the autobiographical sense of identity that projects back into the past and forward into the future. Finally, heightened sensitivity to body and environment occurs with a decrease in the forms of reactivity that create mental distress.

### Brain Training Method: Emotion Regulation

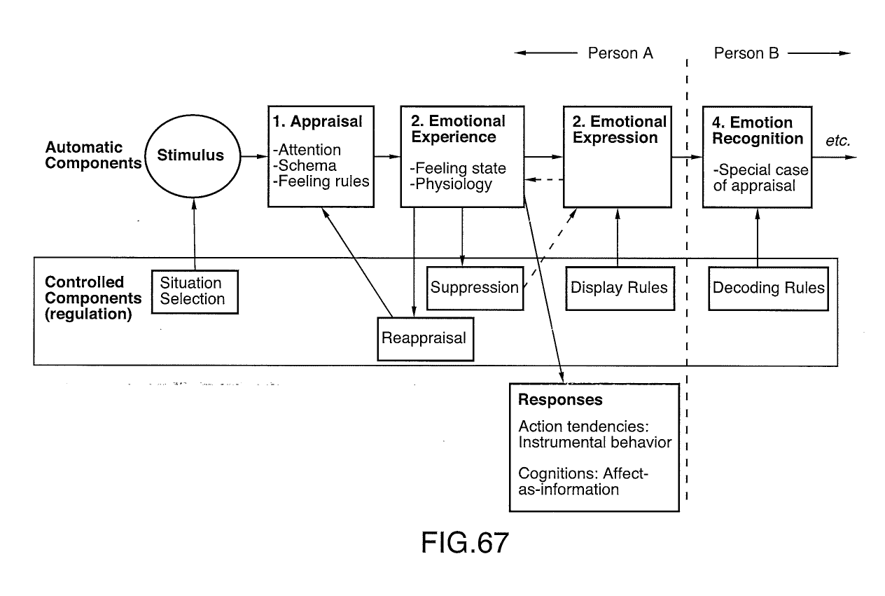

The system of the present invention may provide support for emotion appraisal (arousal/valence) for those that are “emotionally self-opaque”. The system may support for interpersonal exchange, noting that an emotion has been experienced and to prompt expression. An example of a corresponding user flow may be seen in FIG. 67.

### Brain Training Method: Mindfulness-Based Method

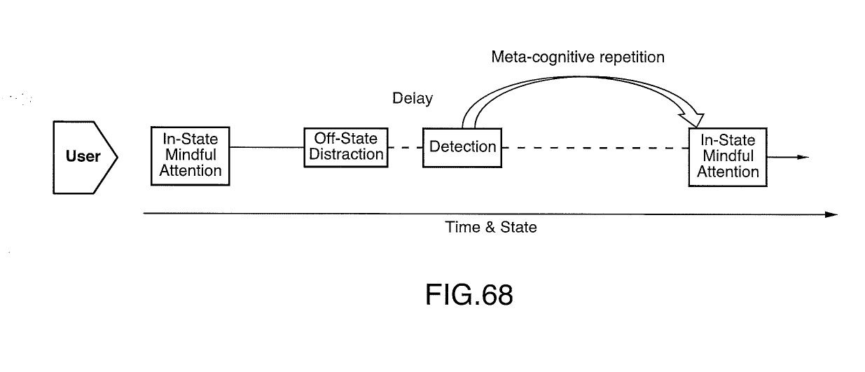

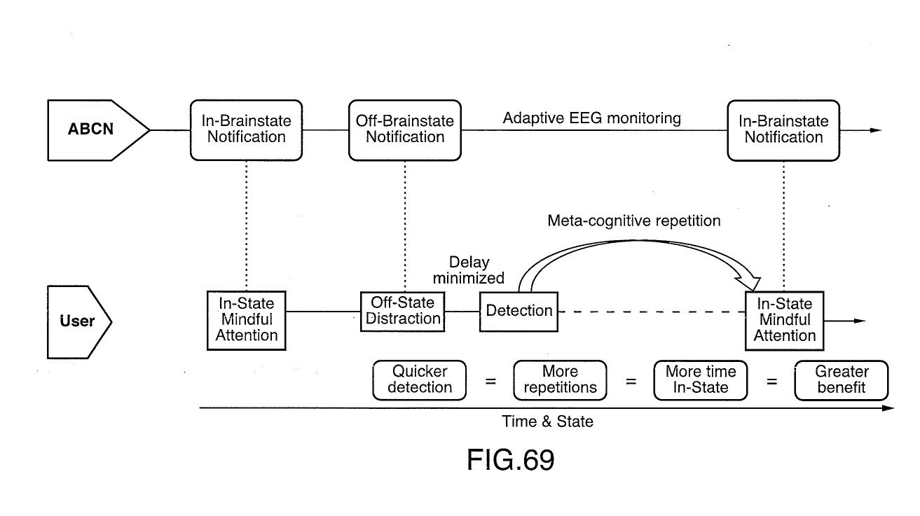

In a Mindfulness-based Method of brain training, the user sets the intention to place their attention within a non-judgmental way upon body sensations, feelings, emotions and thoughts with the goal to familiarize the user to their internal processes and cultivate self-regulation and self-awareness. An ABCN “in-state” may include attention placed on purpose, in the present moment and non-judgmentally. An ABCN “off-state” may include drifting into thinking, mind wandering, anxiety or drowsiness. FIG. 68 shows a Mindfulness-based Method of training, without ABCN, and FIG. 69 shows a Mindfulness-based Method of training with ABCN.

An example of a mindfulness-based exercise script for mindfulness of body sensations may include an introduction part, where the system directs the user to find a comfortable seated position, with both feet on the floor and close your eyes if you haven't already; and to place your hands comfortably on your lap and allow your spine to grow tall relaxing your shoulders. ABCN elements may then be introduced. For example, “while your attention is on sensations in the body, the headband will sense it, and you'll hear this . . . (2 sec) . . . {Stable State ABCN}. At some point, your mind will naturally become distracted and wander. When your mind wanders, the headband will sense it, and you'll hear this . . . (2 sec) {Unstable State ABCN}. If you notice the sounds changing, simply notice this and bring your attention back to the exercise without judging how you are doing.” Body scan prompts may also be incorporated, including for example: “To start, Take a moment to settle into your body by becoming aware that you are breathing . . . {3 sec}”; “Noticing the top of your head and forehead and if there's any facial expression you might be holding or a sense of tightness or release in your brow . . . {10 sec}”; “Dropping the jaw gently . . . and noticing the shoulders, allowing them to drop or relax . . . {10 sec}”; “When you notice that your mind has become active, distracted or you're at times not noticing any sensation, know that this is perfectly normal . . . ”; “Moving onward, bringing into awareness and scanning from the shoulders down to the wrists, and then noticing sensations arising at the hands . . . ”; “Notice the rising and falling of the chest and belly, as you breathe in and out . . . opening up to these sensations of life, sensations of the body breathing without judging how you are doing {10 sec}”; “Noticing your seated position, the connection to the chair or cushion . . . and when you're ready bringing into awareness and scanning from the hips down to the thighs, calves and feet . . . ”; and “Open your awareness to how your whole body feels and continue to breathe for a few more moments.”

MBP has a huge underpinning of evidence in the literature. Evidence for the efficacy of MBP elucidating the long term brain health benefits for its practitioners has seen an exponential increase in the past decade. Health outcomes such as reduced rumination [1], stress reduction [2], boosts to working memory [3], increased focus [4], less emotional reactivity [5], greater cognitive flexibility [6] and improved relationship satisfaction [7] have been observed. Neuroscience research has shown that MBP can change the default behaviours of the brain, through EEG and fMRI studies [8, 9]. Other fMRI studies point to structural changes in the brain associated with MBP such as increased axonal density and myelin thickness [10-12] (see end of description for reference information).

The app of the present invention provides instructions for conducting exercises, usually through a recorded voice, to a user that helps them evoke brain states. In addition, the APP provides feedback to the user of their current brainstate in a process called neurofeedback. The exercises that users are asked to do are based on a combination of brain training methods supported by a specific combination of user established intention(s), user placing attention upon body, feeling, emotion and thought sensations, all supported by a specific set attitude.

The following three mechanisms are used to support within mindfulness-based brain training of the present invention): intention or “on purpose” (supported/motivated by data screens and value proposition); Attention or “paying attention” (supported by Brain Training ABCN paradigm); and Attitude or “in a particular way” (supported by guidance and non-judgmental display of post session Data). These are not separate processes or stages, but are interwoven aspects of a single cyclic process and may occur simultaneously [15].

The intention mechanism may establish relevance and purpose of the exercise to the user's own set of life challenges and experience: self-regulation, self-exploration and finally self-liberation. The motivation for beginners doing this brain training are the extrinsic awards supplied by using the APP and then shifting to intrinsic motivation and application of the system within the user's life.

The attention mechanism may be useful for the user to learn to self-regulate their attention while anchored to the sensations of their breath (object of attention); observing the operations of one's moment-to-moment experience that includes both internal experience (i.e. one's thoughts) and external experience (e.g. external stimuli) picked up through one's senses. The user learns to direct and sustain their attention on a selected object (e.g., breath sensation). They also learn to quickly detect mind wandering and distractors (e.g., thoughts). The user learns to disengage their attention from distractors and shifts their attention back to the selected object. Cognitive reappraisal of the distractor (e.g. “just a thought”, “it is okay to be distracted”) is another skill that the user learns using this Brain Training approach.

The attitude mechanism may be useful for the user to attend the app with curiosity, non-striving and acceptance.

The three axioms of mindfulness (intention, attention, and attitude) are not separate stages. They are interwoven aspects of a single cyclic process and occur simultaneously. Mindfulness is this moment-to-moment process.

The system may support the user within the various exercises to cultivate mindfulness—placing a non-judgmental attention upon: physical sense object; body, breath, sound, sight etc; feelings/emotions; thoughts; volitions.

An exemplary script for mindfulness-based brain training is provided as follows:
Introduction and Instructions
   You are about to do an exercise designed to help you reduce stress, alleviate anxiety, and increase focus and concentration.
   The goal of this exercise is to calm and settle the winds in this environment by calming and settling your mind. You'll do this by focusing on counting your breaths.
   When you're focused, the system will detect steady and constant brain signals and translate them into calm and peaceful winds like this. {ABCN goes to low level wind, but not total silence}
   Eventually you will become distracted, and your mind will wander. When your focus shifts away from the exercise, the system will detect fluctuations and changes in your brain signals, and translate them into strong winds like this. (ABCN goes to high level wind)
   The more you use the system, the better you'll get at calming the winds. After you've completed enough sessions, the system will be able to build a model of your brain, which will enable advanced tools to help you see how you're improving.
   Tap the screen when you're ready to try the system.
Introduction to Focused Attention Meditation (Intention, Attention, Attitude)
   You won't need to look at the screen during this exercise, so feel free to put your device down within reach or rest it in your hands.
   When you're ready, sit in a comfortable position and close your eyes. Allow your back to be straight and relax your shoulders.
   Settle in, take a few moments to relax by becoming aware that you are breathing.
   No need to change your breathing, your body knows how to breathe.
   To help focus on your breathing, count each out-breath up to 10, then start back at 1, and begin the count again.
   When you notice that your mind has wandered or you have lost count, don't worry. Just start back at 1 and resume the count again without judging how you're doing.
   Ready? Begin counting your breaths now.
   {starting sound plays, timer starts, & nfb control of wind starts}

### Brain Training Method: Compassion-Based Method

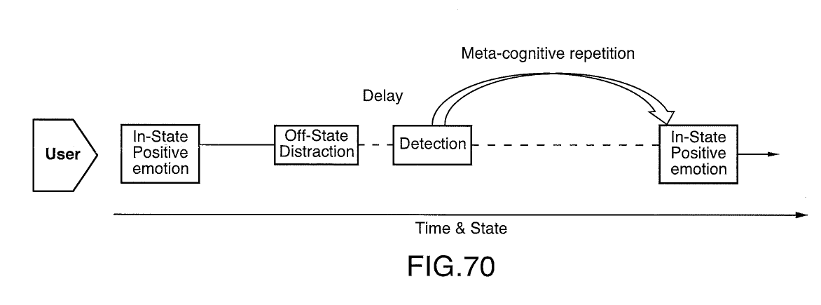

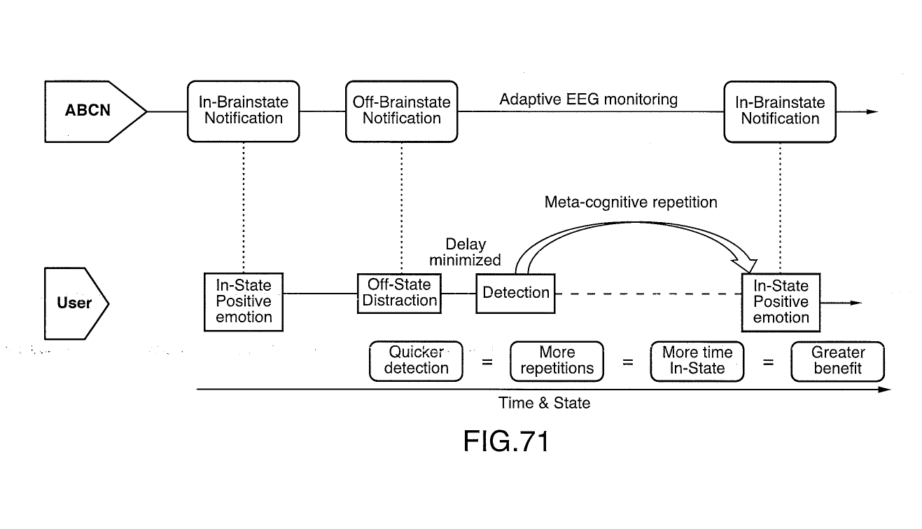

In a Compassion-based Method of brain training, the user sets the intention to generate positive emotions; such as loving/kindness, equanimity, compassion, joy, etc. with the goal to have these positive emotions naturally arise to stabilize negative emotions in moments of interpersonal challenge and to boost positive emotions in moments of joy and happiness. An ABCN “in-state” may include attention placed on generating positive emotions. An ABCN “off-state” may include drifting into thinking, mind wandering, anxiety or drowsiness. FIG. 70 shows a Compassion-based Method of training, without ABCN, and FIG. 71 shows a Compassion-based Method of training with ABCN.

An example of a mindfulness-based exercise script for mindfulness of body sensations may include an introduction part, where the system directs the user to find a comfortable seated position, with both feet on the floor and close your eyes if you haven't already; and to place your hands comfortably on your lap and allow your spine to grow tall relaxing your shoulders. ABCN elements may then be introduced. For example: “While your attention is on generating positive emotions, the headband will sense it and you'll hear this . . . (2 sec) . . . {Stable State ABCN]”; “At some point, your mind will naturally become distracted or begin to get caught up in thoughts about the past or the future”; “When your mind wanders, the headband will sense it, and you'll hear this . . . (2 sec) {Unstable State ABCN}”; and “If you notice the sounds changing, simply notice this and bring your attention back to the exercise without judging how you are doing”. Positive emotion prompts may also be provided including: “To start, settle in, take a few moments to notice how your body feels . . . noticing the sensation of breath entering the body and leaving the body”; “When you are ready bring your attention to someone that you have good feelings towards, and as you hold them in your thoughts begin sending them well wishes, good health and peace”; “Now, as the breath still rises and falls, bring to mind another person that you naturally have good feelings towards and as you hold them in your thoughts begin sending them well wishes, good health and peace”; “Now, bring to mind yourself and as you hold yourself in your thoughts begin sending well wishes, good health and peace”; and “Now, to complete this exercise begin to spread these positive emotions more broadly bringing to mind a community, a species or a part of the world and as you hold them in your thoughts begin sending them well wishes, good health and peace”.

The scientific study of compassion meditation in particular and methods for cultivating it is significant for several reasons, including individual physiological and psychological health, as well as broader social issues of human connection and survival.

Compassion may be important for human happiness and well-being. Practices that enhance our sense of connectivity with others, such as compassion training, might show positive effects on our physical and mental health. Compassion-based training may provide for a systematic practice of gradually training the mind in compassion until altruism becomes spontaneous. Compassion-based Training Method aims to help practitioners progressively cultivate other-centered thoughts and behaviors while overcoming maladaptive, self-focused thoughts and behaviors by moving systematically through eight sequential steps.

The system of the present invention implementing a compassion-based training method provide for: cultivating self-compassion; developing equanimity; developing appreciation and gratitude; developing affection and empathy; realizing aspirational compassion; and realizing active compassion.

### Stimulus as Therapy

There system of the present invention may be configured to interpret or control a variety of sensor or input technologies, including EEG, fNIRS, fMRI, TCMS, electroconvulsive, tDCS, and ultrasound. These terms are generally defined as follows.

ABCN neurofeedback, also called neurotherapy or neurobiofeedback, is a type of biofeedback that uses realtime displays of electroencephalography (EEG) or hemoencephalography (HEG) to illustrate brain activity and teach self-regulation. EEG neurofeedback uses sensors that are placed on the scalp to measure brain waves, while HEG neurofeedback uses infrared (IR) sensors or functional magnetic resonance imaging (fMRI) to measure brain blood flow.

FNIRS functional near-infrared spectroscopy is a form of neurofeedback (HEG) for the purpose of functional neuroimaging. Using fNIR, brain activity is measured through hemodynamic responses associated with neuron behavior.

fNIR is a non-invasive imaging method involving the quantification of chromophore concentration resolved from the measurement of near infrared (NIR) light attenuation, temporal or phasic changes. NIR spectrum light takes advantage of the optical window in which skin, tissue, and bone are mostly transparent to NIR light in the spectrum of 700-900 nm, while hemoglobin (Hb) and deoxygenated-hemoglobin (deoxy-Hb) are stronger absorbers of light. Differences in the absorption spectra of deoxy-Hb and oxy-Hb allow the measurement of relative changes in hemoglobin concentration through the use of light attenuation at multiple wavelengths.

TCMS/TMS is transcranial magnetic stimulation. Transcranial magnetic stimulation (TMS) is a procedure that uses magnetic fields to stimulate nerve cells in the brain.

With TMS, a large electromagnetic coil is placed against your scalp near your forehead. The electromagnet used in TMS creates electric currents that stimulate nerve cells in the region of interest in the brain

RTCMS/rTMS is repetitive transcranial magnetic stimulation (rTMS).

tDCS is transcranial direct current stimulation, which is a form of neurostimulation which uses constant, low current delivered directly to the brain area of interest via small electrodes

Ultrasound refers to ultrasound waves that are used to effect brain activity.

Each of these technologies may involve reading and stimulation of the brain to change the response of the brain. The method of the present invention in general has four steps: (1) acquire signal, (2) analyze, (3) interpret and (4) present notification or stimulus to the user. In addition to passive notification like audio feedback, the present invention may also provide stimulus as a therapy to the user. Although EEG neurofeedback is discussed throughout this document, the present invention is not intended to be limited to any particular type of sensor input or stimulus type. tDCS could be substituted in most of the paradigms, for example, with the tDCS triggered when wind happens, for example. The system may stimulate your brain for you rather than you having to stimulate it yourself.

In the case of EEG neurofeedback, the system may read the user's brainwaves, measuring against some norm or optimum, and then rewarding the brain (through visual, audio, haptic feedback) for moving itself towards that optimum brainwave pattern.

In the case of Fnirs (a form of HEG), the system may read the hemodynamic response of the brain, often measuring against a norm, and rewarding it to move towards an optimum.

In stimulation therapies, the system may read the state of the brain, often measure it against some norm, and then apply a stimulation modality—electric, magnetic, or ultrasound, to move it towards an optimum. With stimulation therapies, the system does not strictly have to read the state of the brain before stimulating, but effectively applied therapies likely would want to.

### Brain State Guidance Applications

The system of the present invention may be used for depression therapy, by changing the algorithm, optionally by rewarding a paradigm that improves depression, for example, correcting alpha asymmetry via the two frontal channels. Frontal alpha asymmetry is an indication of depression. Amelioration of depression through rewarding frontal alpha ratios that move towards a normal brain. As in ADD, an algorithm and neurofeedback paradigm that train a normalization in frontal alpha can be substituted for the algorithm that detects and trains meditation.

The system may be used for ADHD therapy. Children with ADHD have been shown to have low beta:theta ratios. Neurofeedback paradigms that increase beta and decrease theta can have therapeutic effect in reducing ADD and ADHD symptoms. For example, a game could be a driving game, in which the speed of the car is driven by a user's beta/theta ration, and achieving the correct beta/theta ratio could drive the car forward. Swapping in an algorithm to detect beta/theta ration for the algorithm used to determine state of meditation, is a linear swap. The content that we have generated for a meditation training tool can also be applied for an ADD tool. This is the case for two reasons: (1) Beta/Theta ratio or a Slow Cortical Potential-based algorithm (SCP) can be used instead of our meditation algorithm for ADD/ADHD neurofeedback; and (2) Our meditation algorithm and the act of meditation increased Attention, and is itself a form of attention training.

The system may be used as a diagnostic tool. Beta/Theta ratio has been approved by the FDA as a diagnosis for ADD. In the same way, the application of the present invention could be used as a diagnostic for attention/meditation capabilities. For example, response to audio or visual EPR such as n200 or p300 could be used to diagnose attention capabilities. The greater the EPR, the more attention the user has paid to the stimulus, and the better the users “attention”. Frontal Alpha has been demonstrated to be a predictor of ability to learn videogames. An increase in frontal alpha may indicate an increased ability to learn videogames. In this way, frontal alpha can be used as a diagnostic for learning ability. In an implementation of the present invention, there could be a “learning score”, indicating the user's propensity to learn something quickly. As the user engages in alpha neurofeedback and increases frontal alpha, this learning score could go up. Someone learning a task could be rewarded for high frontal alpha initially. High frontal alpha could be used as an input to an engine that determines the difficulty level of a game, and higher frontal alpha could mean that the game's difficult progresses more rapidly. High frontal alpha could be used as a discriminator for hiring someone for a task or a role, particularly one that requires attention and fast learning.

In an implementation, the system may be configured to synchronize high attention to alpha phase. Attention comes on-line in pulses that can vary with alpha frequency, maxing every 11 ms or so (as alpha frequency is 8-12 hz). Stimulus presented in during the peak amplitude of alpha waves are attended to. The system of the present invention may be configured to time-lock information to be remembered/learned/attended to the peak frequency of alpha (e.g. presented at a time that coincides with alpha peaks (every 11 ms (or so)). This may allow for greater learning of or attending to that information. One way the system could operate is that a stimulus (audio or visual) is rhythmically presented every 11 ms (or so) to entrain the alpha frequency. After a minimum number of presentations sufficient to entrain alpha frequency in the brain (min of 10), relevant information is then presented in phase with the alpha entrainment. This could also be used to diagnose the propensity for something to be seen, by checking a participant's alpha rhythm and proactively or retroactively determining if a stimulus was attended to based on whether it fell during alpha peaks.

This phase-locking alpha effect can be used in advertising, meaning that an advertiser wishing to gain greater attention to their message could use the above method to entrain alpha waves, and then present information at times that correspond with positive peaks in the alpha wave. It could also be used to scramble messages, by entraining an alpha rhythm and then presenting information during the troughs in alpha amplitude.

One could be rewarded for maintaining high levels of alpha while performing a cognitive task, like a digit span task, or a sustained attention task. One could be rewarded for maintaining high levels of beta in the situations above.

A user's attention level may be diagnosed during a game by the system. For example, the system could do dual N-back or other auditory test to diagnose attention abilities. For example, measuring amplitude of p300 as measure of attention.

Looking at changes (variability) in state may be implemented in a wide variety of diagnostic and treatment methods, including: ADD; Depression; Anesthesia and consciousness monitoring; detecting changes in Coma state; Alzheimer's; TBI diagnosis and treatment; Bipolar disorder; Anxiety; Stress; Rumination; Sports training; Cognitive enhancement; Attention training; Addiction; Obesity; Smoking Cessation; Anger Management; Epilepsy; Insomnia; Autism. In this case, it is the application of the method of looking at the jumping between states (Chris Method) that lets us see the brain dynamics in these different “disease” states in unique ways that let us classify them as different than a normal brain, and apply neuromodulation therapy to correct them to normal. For example, for smoking cessation, the system may be configured to look for the Neural Signature of “smoking desire” and train the brain against that.

In another treatment mode, the system of the present invention with its meditation training algorithm, can be applied to various “diseases” including: addiction, obesity, smoking cessation, anger management, OCD, anxiety, anorexia, etc. In these cases, the emphasis is on the application of meditation like techniques and meditation-like brainwave characteristics in the amelioration of the diseases. For example, in diseases of addiction, using meditation to learn to, for example, appropriately resist turning sensations of craving into the act of smoking, is one of the modes of action.

The system of the present invention may also be configured to act as a sleep aid, by training a user with 13-15 hz rhythms during the day (mu) in order to help the user sleep better at night, and improve latency to sleep.

### Application: Tracking and Treatment

The system may track the user's brainstate over time and that information may be used to drive treatment options for the user. The brainstate of employees can also be tracked to inform a manager about his/her options.

For example, employees do brain fitness exercises using the system of the present invention based on neurofeedback. Employee may self-monitor for stress, focus etc. Long term tracking of mental correlates are also tracked. Brain data builds business case for additional coverage by insurance. It also allows testing of wellness programs and productivity solutions. This can also be expanded to team based training of emotional intelligence.

In another example, children with learning disabilities/disorders may use the system to track their progress through school. Improvements are suggested through therapy, additional tutors, adjusting medication dosage.

In another example, the system may track the user's brainstate in a Yoga Session. Tension in mind is related to tension in the body. User can more easily release tension if they are in the right frame of mind. Guidance can be given to improve self-visualization exercises.

In another example, the system may monitor a student's progress. The system may monitor how students feel during taught material to know when to intervene and what to focus on. For each student determine time of day when they are most receptive to learning. This will help teacher determine when to teach difficult subjects, e.g. teach math in the morning or afternoon. The system may monitor how well students are doing (online e-learning). Course material may be modified in response to be more difficult or easy. Special needs students may be monitored for early detection of an episode. An instructor may see the effectiveness of course material across students in class and adjust content and learning style accordingly based on the brain state responses of the students.

In another example, it can be therapeutic for a user to understand the emotion(s) they are feeling. This could occur in a therapist/patient setting or within a support group. Emotional intelligence could be taught by compiling the statistics of emotions across a classroom in response to scenes or photographs. In addition, it may be helpful for people to understand the emotional impact they are having on other people. A therapist could monitor a couple during therapy sessions to ensure both parties are engaged while listening to each other.

### Application: Content Recommendation and Curriculum Control

The system may include a recommendation engine which may recommend exercises to the user based on a variety of information, including: preferences of user (e.g. what colours relax them); user-reported mood, energy level and other information; goals the user has selected for themselves; input from a teacher, doctor, parent; physical activity data and other data from other sensors. The system may recommend a meditation exercise based on discovery stage or user choice, past performance, and other factors, including, for example, time and location data from GPS (e.g. on your morning commute to work it may recommend a “get up and go” training; at home at night it recommends the “unwind and relax” program). The recommendations may be organized in a manner similar to a play list that allows the user to either choose the recommended training, or optionally skip ahead in the play list, or choose something on their own.

Exercise recommendations may be made based on the time of day, or the exercises may be customized based on the time of day (e.g. varied visuals/environments). A way to note and measure your life goals with the system and then check in on your progress toward those goals. A “place” which you cultivate as you progress with your system (your ‘trophy case’ is a growing and developing place of some kind, like a garden). An interactive graph may be provided by the system to allow the user to listen to parts of session. Browsing through previous sessions, each session may be represented by an “infoviz” icon which reflects the nature of that session (i.e. a flower who petals' shape and colour reflect the brainwave attributes of that session). The user may voluntarily opt-in to random notifications at custom frequency to remind users to check-in (e.g. consider their current brain state even when not using the system). If the user doesn't use the app for a while, the system may be configured to see some kind of depreciation of the user's score or of the app.

An advanced calibration mode may be provided which allows users to identify the mental attitude that's “holding them back”, and then do a screen-tap noting procedure to identify moments when they are experiencing that attitude (i.e. impatience, dullness, etc.). This may assist in strengthening the user's data profile and also provide more responsiveness to them in their experience. The system may be configured to provide for the ability to leave a marker during a session and see it when you're looking at your data. SMS experience sampling may be correlated with sessions, can be displayed among sessions as additional data on the user's state. The system may adapt difficulty of exercises over time. The system may determine whether sessions are too easy or hard and unlocks difficulty tweaks (e.g. −10% or +10%, etc.).

Accordingly, environment recommendation may be based on previous performance brainstate, and real-time environment selection may be based on adaptive brainstate classification. A recommendation of specific target brain state exercises based on previous performance and usage history may be provided by the system.

A specific sequence of target states could have profound effects. The system may be configured to build training programs out of a sequence of target state modules. For example, an application with only a few target state modules could offer more training programs which each represent a certain sequence aimed at a specific user need, such as in a “Morning Power-Up” training program starting with a 2 minute calm state, followed by a 2 minute focus state, followed by a 1 minute positive emotions state.

### Application: Social Media

Information obtained or generated by the system may be shared with other users or friends, optionally through the app or through a social networking platform such as Facebook. Two friends in a social network can perform the same practice at different times and hear in conjunction with the practice how their friend did at various time for example as means of encouragement or motivation. Group meditation sessions may be organized. Users may view one another's profiles, or a profile based on normalized score based on demographic traits (such as average North American male). Users may create and upload their own meditation practices for others in the community to download and try. Users may build and participate in communities of like-minded meditators. Through sharing information from the system, users may see how other friends are performing with the system, and see which friends are using the system right now. An avatar representation may be used to represent a user's unique accumulated emergent properties (i.e. a user who has collected mostly birds and waves is represented by a water-bird of some kind). The system may be used to connect 2 friends in real time/ability to do a session with a friend (where they're represented by audio avatar). Optionally, the system may connect the user with other users not known to the user. The system may provide for the ability to “give” a badge to someone. The badge may be lost after giving, adding value to the badges and gift, and providing an endless incentive to keep earning badges. Messages may be submitted to accompany the gift. Users can share gifts using device native “activity” window which throws a link to other apps. The system may optionally provide a connection with charity/non-profit where users' meditation thematically leads to donation (e.g. collecting birds in a brain state guidance exercise may go to bird conservation; engaging with water leads to donation to clean water charities, etc.).

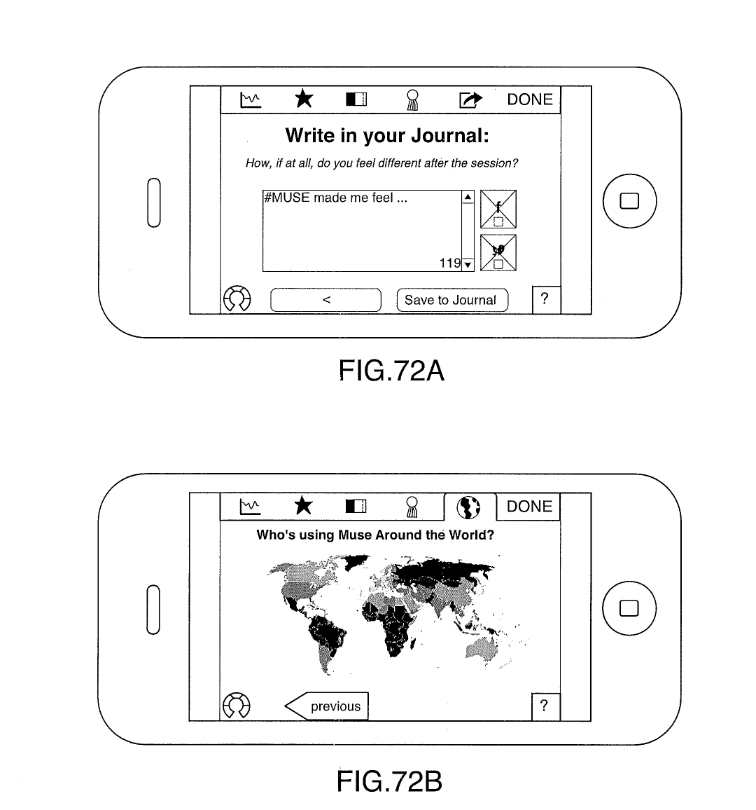

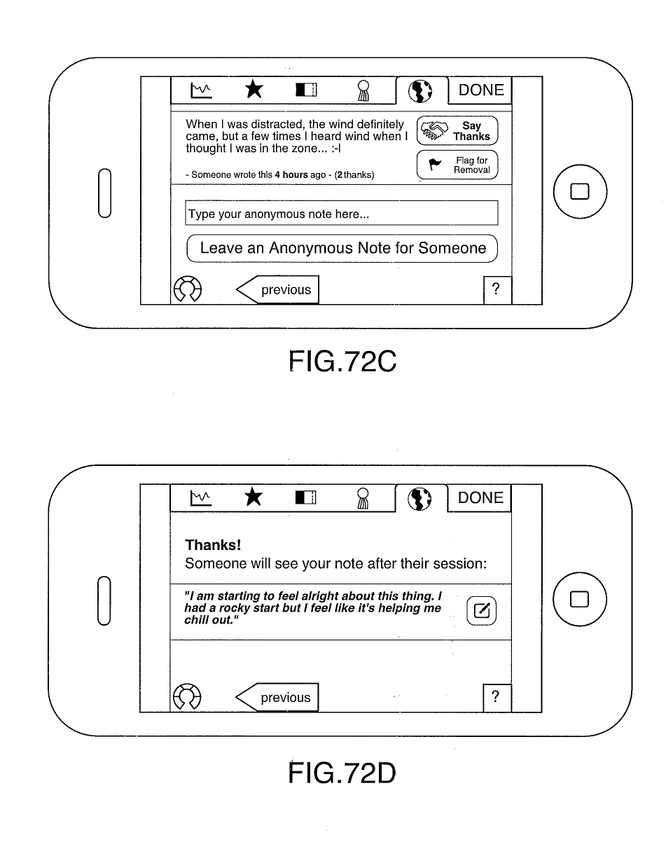

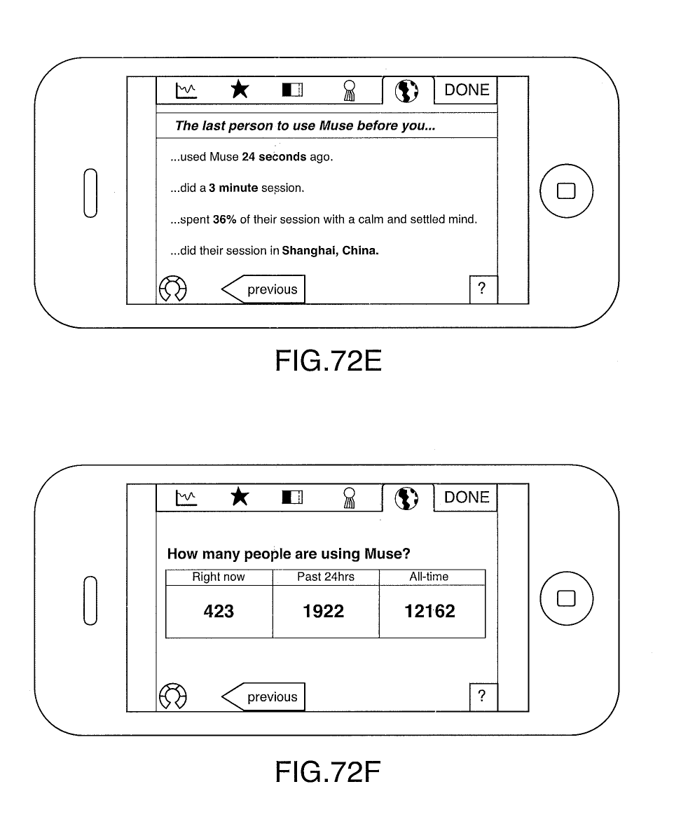

FIGS. 72A-72F show exemplary “sharing” interfaces of the system of the present invention.

### Application: Using Error Related Negativity as a Training Method

An Error Related Negativity (“ERN”) is an Event Related Potential (“ERP”) that occurs when someone perceives they have made an error. ERNs can be used in the context of a therapeutic application. In this context, the presence of an ERN can be used to indicate negative self-judgment. The presence of an ERN at an appropriate place in the application of the present invention (e.g. after a difficult task, or after mind wandering), can be a trigger for the application to engage a mechanism to defray this harmful negative self-judgment. It could trigger a soothing voice to come on to remind the user that everything is OK, or that he can let go of negative thoughts. Or it can play a disrupting or distracting tone to interrupt the negative thought and de-program it.

In a therapeutic context, this is often referred to as “quieting the inner critic”, and is a key step in decreasing negative thoughts and increasing productivity, confidence and self-image. Using an ERN would in a sense be automating that process using brainwave detection technology.

Alternatively, ERNs can be used to diagnose whether a user has an appropriate grasp of their own abilities. When doing a task, and ERN will fire when the user perceives he has made an error. The time when a user perceives he has made an error and the times the user has actually made an error can be matched up to indicate whether a user has appropriate self-perception of their errors.

The size of the ERN can also be used to set the difficulty level of a game or cognitive training task, so if the user perceives themselves to be very incorrect, the game can get easier, a piece of information that can be taken into account along with a score to determine the appropriate level of a next trial.

Using ERN may assists to help a user in learning. ERNs fire when you have made a mistake or detect a mistake in the environment. These might be obvious mistakes, but ERNs also fire when you have a detected a mistake, but not obviously (such as “subconscious” detection of a mistake”.

This has relevance in applications where there is a minor mistake in the environment that you take note of, and a tone or signal triggered by the ERN turns the experience into a major error that you learn from.

For example, you are copy editing, and you are able to find mistakes more effectively—you have an ERN when you notice a mistake. This will allow you to notice a greater percentage of mistakes. This also trains you to be a more effective copy writer.

A security analyst looking at satellite surveillance photos can be assisted to find something salient only to their subconscious, or pre-conscious by analyzing their ERN response to a set of photos. Satellite surveillance photos may be annotated by image recognition or ML. There is a machine learning that uses image recognition to classify objects. Often this is not accurate and the human can find errors that ML made and allow human to more efficiently label ML errors. This will increase classifier performance.

Doing coding and helping coder realized their errors. It trains you at being better. This allows for more efficient and guided learning. Possibly confidence because person realizes they can identify errors. Balance is needed by user preference not to overwhelm user with unwanted errors. Often people cannot put a label on their error but have a gut feel.

There may be a different ERP that occurs if the error was not made by the user and detected in the environment versus an error that the user made. This will help mitigate the damage large amount of error reporting has on a person's confidence.

ERN coupled with asymmetry indicating dislike. In interaction design, the APP behaves in a n unexpected way and this can be detected by ERN plus asymmetry and can be used to help interaction designers refine their designs.

ERNs can be used as a means of making people aware of or help them make use of what is often called “intuition”. The idea that you have a “sense” that something is wrong. ERN helps you to identify when you have the “feeling” that something is wrong, and then act on it.

### Application: Medical Applications

EEG signals along with other biological and non-biological data can be used to diagnose medical and or psychological disorders. Rules used for diagnosing can be learned from the data stored in a computer server implemented in the cloud. In addition these rules can be obtained from Clinical Practice Guidelines or other evidence based medical literature.

### Application: ADHD Diagnosis

Analysis of EEG signals can be used to classify a person as ADHD or not. A person undergoes a prescribed set of exercises while their EEG signals are recorded. The system takes raw EEG signals from multiple channels across the scalp and removes unwanted artifacts from the signals and replaces them with de-noised EEG. A Fast Fourier Transform applied to each EEG signal and theta and beta band power is determined. The ratio of theta/beta power is compared to a ratio and if it exceeds the ratio then the person is diagnosed as ADHD. Enhancements to the invention include using transformations of the EEG signal to emphasize the contribution of some EEG channels over the others and to consider the contribution of spatial patterns.

### Application: ADHD Therapy

In another possible implementation of the invention, a computer system may be provided for ameliorating ADD or ADHD symptomology. For example, a user (adult or child) may see his/her level of alpha, beta or theta waves as recorded from a 1, 2, 3 or 4 (or more) electrode system, while he is performing an everyday task. The information can be displayed on a smartphone, tablet, computer or other device. The information can be presented visually in the form of a graph, table, pictorial or rating system. It could also be represented audibly. In one possible implementation the form of a sound for example its pitch or volume may be changed by the computer system as brain state measure changes. These changes could also be represented through vibro-tactile feedback.

This information about his/her brain state could be used to understand the user's level of ADD or ADHD symptomology, and the user could use the system to be encouraged or rewarded to remain in the state of high beta wave or high alpha wave or upper alpha, and low theta wave. The system may also implement SCP and gamma effects.

Output of real-time measures related to the user's brain-state and or brain-wave phase, will enable applications that can modulate the presentation of stimulus so that we may want either to emphasize or deemphasize its effect on us.

For instance timing the switching of images so that we are most able to remember them. The brain state guidance exercise provided by the system of the present invention could be an audio book or media player that stops advancing when we are not paying attention. This can also be used to challenge people to stay focused on sensory input (much like the attention paced audio book), to give the user a mental workout, particularly during activities that are not typically beneficial to the mind (e.g. when watching tv). The system could configure a media player could change the channel characteristics, including, for example: improved contrast; presentation of advertising; change the frequency response of the audio or visual channel; and beamforming to accentuate a particular spatial domain. Stimulus modulation can be used to helps people snap out of laxity, or self narrative, through highlighting sensory input.

The system may include a rules engine that may let the system know as per user settings what it should be emphasizing vs. de-emphasizing. The user could have a distraction knob, that uses adaptive filtering to optimize based on user setting: e.g. know may go from Isolation->inspiration->distraction.

### Application: Epilepsy Monitor

In another possible implementation of the present invention, an improved epilepsy monitor system may be provided. In this aspect, an epileptic patient wears an EEG headset that records their brainwaves throughout their day and streams them to a client application on a smartphone for example. In the event of a seizure, the application may for example notify automatically the patient's doctor, contact emergency services via internet connection, and/or provide context information such as GPS and auditory environment, and brainwave history pre and seizure as well as real-time EEG data.

### Application: Quantified Self Integration

The present system may be integrated with other quantified-self products such as the Jawbone UP, Fitbit, BodyMedia, heart-rate monitors and the like. For example, data from these third party systems may be obtained and synchronized with data created by the present system, for example its MED-CASP system implementation as described. In one implementation, the system of the present invention may transform ambiguous intangible (to a user) brainwave data to clear tangible information which is then processed and transformed into info-graphics and self-metrics serving as a tool to measure and increase an individual's human capital. The system would be designed for use in everyday life, recording a user's brain-state over the course of an entire workday. At the end of the day, the user would download that day's data into a computer, which would analyze it and compare it with previous data looking for patterns. The computer would prompt the user to enter details about the day's activities, and then correlate them with the user's brain states.

The system of the present invention may be configured to output data, including EEG data, to a data aggregator such as MyFitnessPal™ which may be operating in the form of an application on the computing device or may be or may be operating on a separate computing device.

The data output to a data aggregator may include, for example: the amount of time spent in a particular brain state on particular days, or during particular sessions; the number of times a brain state guidance objective was achieved; an identification of brain state guidance objectives achieved or gamification rewards earned by using the system of the present invention, the amount of time spent using brain state guidance exercises; percentages of time spent in various brain states; and any other data related to the present invention.

### Application: Distraction Monitor

In another possible implementation of the present invention, the computer system of the present invention may be configured to act as a distraction monitor. This may be used for example to improve the social aspects of telecommunication, to put user in better touch of the cognitive state and level of distraction of person they are talking with. For instance, this would allow for cognitive state feedback of a cell phone user that is driving. The system would be able to inform the other user of the relative balance of attention between the conversation and external factors (this happens naturally when the people communicating are in the same spot).

### Application: Cognitive Training EEG System

In another possible implementation, a user wears a soft-band brainwave headset that is connected to their computer or mobile phone via BLUETOOTH™ while they execute cognitive training exercises that are modulated by target brain-states. The design enhances in-domain problem solving through practice and facilitates crossover through target state association.

For example, in one particular implementation a Math problems game may be provided that utilizes EEG for scoring and difficulty scaling for example. For scoring, target brain states may be rewarded using point scaling and A/V embellishment. Problems may be provided where EEG controls difficulty (to make easier when in target brain states) through increased information to aid solution (like making hints visible), through the selection of easier problems, modulating time constraints.

In order to enhance skill crossover, appropriate brain states may be linked to incentives or rewards. Selection of these states may be based on cognitive training research with our inventive step being related to how these brain states are incorporated into a game environment. Examples of target states may include: sustaining a beta brainwave during problem solving such as math or memory, seek and find, and matching tasks for example; or sustaining alpha brainwaves while doing mental rotation. In all tasks beta brainwaves may result in a change in the level of difficulty of the game, affect audio or affect something on screen.

### Application: In-Flight Entertainment System

In yet another possible implementation of the present invention, an in-flight entertainment system may be provided that combines brainwaves input, and sound/visual output on an airplane. Biofeedback may be provided to make travelling by air more pleasurable to passengers. The system may be configured to help people relax, and control anxiety.

### Application: Compare-Your-Brainwaves

In one possible extension of the present invention, users wear a brainwave headset connected computing device, a sample of their brainwaves is taken, while resting or while performing a certain activity. These brainwaves are then uploaded to the cloud network, where a cloud implemented resources implements one or more processing algorithms for rating the brainwave samples according to several criteria. The results of this analysis may be compared with that of other users, either friends from a social network for example, random users from the public or celebrities, who collected their brainwave data in a similar test. The comparison data is then sent back (in one implementation) to the client application on the mobile device, where users can see how similar they are to the other person.

### Application: EEG+Augmented Reality

Salient events could be captured in the User Profile. ML could be applied to learn new things about the person. Use of visual feedback for ABCN may be desirable however it is not very compatible with real world applications since the user is typically using their vision to achieve a primary task and having to look at a screen disrupts this. Augmented reality (“AR”) generally refers to technologies where the users field of view (FOV) is augmented, or otherwise changed, by computer graphics. In this way, AR would allow a user to receive biofeedback naturally, in a way that that can be integrated into many different experience and applications. In one implementation, an augmented reality system may be provided that includes or links to the computer system of the present invention. The augment reality system may include a wearable display (often called a heads up display). An augmented reality system based on the present invention may include identification of brain states, specific to the AR context, that utilize context information from the inertial tracking and the computer vision processing done within the partner companies' aspect of the application. This may include, for instance, the identification of P300 waveforms time locked to visual events in the AR field of view.

The computer system of the present invention may be configured to use ERP or other BW feature to determine if there are visual events in the user's FOV that are significant. Surprising or significant occurrences such as seeing a car that the person likes, or a threatening event that could cause the computer system to go into quite mode and allow the user to concentrate more on their environment rather than the computer world. Noticing people's faces that the person recognizes or wants to remember. could start a database for a new individual that the person has met, or will meet. may take pictures or start recording of video if the visual information is salient/interesting to the user.

Other applications can also be built that leverage the features of the technology directly. For example, an application may be built that allows a user to see another person's brain state rendered in real time emo-graphics (perhaps around their head or body) to enhance social interactions over low (e.g. lower than real life) bandwidth channels, or to enhance interactions between strangers or between people who speak different languages.

For example, looking at person you are conversing with or perhaps people in a group who are wearing the system of the present invention. People who are upset could have storm clouds rendered above their heads, or people who are happy could have sunshine streaming from them. People who are thinking could have the gears turning rendered above them with computer graphics. People who are relating social to others in their proximity could show lines of interaction between themselves and the people that are relating too (lines of coherence between people). It would be useful for some interactions to show the coherence between the users brainwaves and another, (both the people who the user is communicating with or perhaps even those with whom they are relating but without direct verbal contact).

### Application: Software Development Kit (SDK)

A software development kit may be provided to allow third parties to develop applications using the system of the present invention. It may enable third parties to access to the raw brainwave data, plug in new algorithms for DSP processing, plug in new algorithms for the interpretation of the DSP data, and display monitoring data as to the current state of the headset, FFT algorithms, DSP algorithms, and application-level algorithm data.

### Application: Team-Based Applications

Information about teams and results can be stored in User's Profile. A team (e.g. group of users with a headset device) can be formed around a common goal or principles. Teams could, for example, participate in contests, collect awards, and participate in simultaneous live events where groups compete or collaborate with each other. For example, in one application, a team may collectively collect points toward a goal. Teams have common properties such as a team name, description, list of team members, apps they participate in, individual rankings for each app, and overall team standing versus other teams. A facility is provided for individuals to search for teams and for teams to advertise themselves. A single team can participate in multiple applications. An application may be simultaneous(live) or asynchronous and run either disconnected from the Internet or connected. A disconnected app could be run on a local wired or wireless network. A team member can view the collective data for a team as well as view information about other participants. A database structure for storing data is shown below.

A shared experience may be led by a facilitator. Sessions and results can be stored in User's Profile. Participants may join a “room” which connect participants together. One of those participants is a facilitator or “group leader” (GL) who guides the group on a shared experience. To aid the group leader in his/her task, a dashboard is displayed on the GL's device which allows the monitoring and/or communication with each participant. The group leader can send messages to the participants (such as text, voice, headset data) either publicly or privately. The GL can create or delete rooms, allow participants into the room, or remove them from the room, or set rules for the room (including but not limited to: anyone can join, need a security token to join, whether the session is being recorded, allow user-to-user communication, allow public broadcasts messages from participants, allow participants to see identifying information about other participants, give one or more participants permissions to manage the room and/or participants). The group leader can be connected to more than one room at a time. Each participant is able to monitor and/or communicate with the group leader. A participant can be connected to more than one room at a time. A participant can: upload live brainwave data or previously recorded brainwave data, can choose to expose identifying information to other participants, send private or broadcast messages to other participants. This functionality could be implemented using a server on the Internet as a relay device, or the application could be run disconnected from the Internet over a local wired or wireless network. The server may be used as an additional source of processing power to aid in the group experience, such as generating additional display data.

### Application: Sleep Monitor/Aid

Sleep results and analysis can be stored in the User's Profile. In another possible implementation, the computer system of the present invention may be adapted to provide an overall solution that adapts sleep algorithms to particular users. It may offer functions such as: environmental control of music/lights (that may dim or turn off while when the user falls asleep); working with a meditation trainer application as part of the sleep therapy; real-time sleep competitions with friends to incentivize night owls to go to sleep earlier; Use the collected EEG data to create artistic representations of our unconscious hours; and provide specialized algorithms to facility dream states and lucid dreaming.

### Application: Social Applications

The system may provide for the ability to share a neurofeedback visual and auditory experience with others. This could be done synchronously in real-time in a one to one fashion or in a broadcast fashion (one to many) or it could also be done in a multi-way fashion (sending to many and receiving from many in a group). This could also be done asynchronously, where the user can send or others can browse and choose to view someone else's shared experience stored in the cloud. There are multiple network configurations for this type of social engagement between users, and it could occur via direct peer to peer connection, via local network or via the system cloud infrastructure.

The general architecture for this backend model is similar to a publisher subscriber design where each node in the network (an individual user) is both a publisher of content as well as a subscriber to other users or the cloud.

### Application: White Labeled Platform as a Service

Backend system may be designed in a modular fashion such that each module has configuration and meta parameters such as versioning so that it can be partitioned and subselected for release to client as a platform. Meta parameters such as sharing or sdk level would allow us to selectively share particular features and identified algorithms with partners or keep them only internally. This has implications for a tiered SDK as well as tiered cloud server API.

### Application: Experiment, Research and Scientific Study Tool

Results learned in studies can be used to update User's Profile based on the information in their profile. I.e. based on your brainwaves and other information you are at a higher risk for developing disease X.

The cloud needs to support conducting medical, anthropological or social studies with the data contained in the cloud. A human can design a study protocol and select study parameters. The study protocol and its parameters can include the definition of the population to be studied (e.g. ADHD diagnosis compared to normal), the type of study to be conducted (e.g. randomized clinical trial (RCT), prospective or retrospective longitudinal cohort studies, cross sectional studies, pilot studies or other observational or clinical type of studies. A software based controller in the cloud can then execute operations for querying, selecting users, randomization to belong to different study arms (e.g. sham versus neurofeedback). If the study is prospective or a comparison type study (e.g. RCT of different algorithms or treatment effects) then instructions may be given to users specific to each arm of the study for them to follow. The controller can then provide alerts to users to ensure that they are complying with the study protocol. Then the controller can gather the data relevant to the study, anonymize any identifiers and build a data set that can be analyzed offline by a human analyst or automatically online by study software built-in the Cloud platform.

The backend cloud system is also designed to serve as an external and internal research tool. For internal research the cloud system will serve as a driver for NB testing with subsets of the user population for algorithm or application or user experience experiments. This testing infrastructure will also be useful for external clients or researchers looking to run targeted experiments on specific cohorts within the general population, such as a focused attention test for ADHD patients in a given age range vs a similarly aged control group. An example of this type of configuration is shown below in Figure Group Experiments.

### Application: Affect Sensitive Cell Phones

User Profile can provide information to drive mood enabled apps. In another possible example of use of the present invention, a mobile phone may be modified, and integrated with for example the MED-CASP system implementation of the present invention in order to (A) selects ring-tones and (B) scale ring volumes to match our emotional state and level of distraction. This would enable the system of the present invention to behave like a private secretary who is tuned into our mood.

### Application: Affect Controlled Music Player

In another possible implementation, EEG based affect measurements may be linked with music libraries, and these may be tagged for mood transition probabilities of specific music. One implementation of a computer system configured using the present invention may enable a user to choose a target brain state or mood, and the computer system may generated automatically a playlist of songs associated with will facilitate a brain state transition or state reinforcement.

### Application: Radio DJ Feedback Machine

User's Profile can be updated to record their experience and the audience at the time. In another possible implementation of the present invention, computer systems may be provided that enable improvements to interactions with an audience. For example EEG based audience music feedback may be used by a particular implementation of the present invention for content control and selection. Listeners to a radio station (online or broadcast) using a mobile device enabled with functionality of the present invention for example the MED-CASP implementation described above. Processed brain-state data may be sent to the cloud, where it is shared with the DJ on the radio station. The DJ is able to view all of the data from these users and to understand how the users are responding to his or her musical selections. From there, the DJ can adjust future selections or share that information with the audience.

### Application: Cloud-Based Movie Experience

User's Profile can be updated to record their experience and the audience at the time. In another related example, an entertainment system may be configured to (A) obtain statement of mind or mood information from an audience, and (B) adapt entertainment content (for example story ending) based on this information in order to achieve maximum effect.

Application: Show Teacher which Students are Paying Attention

In another possible implementation of the present invention a education monitoring system may be provided where students in a classroom are each wearing a brainwave headset which is monitoring their state of attention. The teacher is able to access their brainwave data from a dashboard at the front of the classroom, and can use it to monitor whether students are concentrating on their work or not.

### Application: Guidance, Feedback and Teaching of Users

In an implementation of the system of the present invention, the user chooses a goal they wish to reach. A Guidance system in the cloud will then select a teaching program that is customized to the user's specific level for the goal in mind. The Guidance system conducts a survey to help the user clarify their goals and then choose an EEG acquisition protocol to help characterize a patient. A decision tree is consulted that maps where the user currently is and then recommends a learning program for the user. The learning program can include elements of Neurofeedback plus other exercises for the user to complete. The Guidance system can track a user's progress and provide encouragement, advice or alter the teaching program as required. The system uses EEG and other biological data to understand the user's emotional state and adjust the challenge of the learning paradigm according to the user's skill level. This is based on a theory of state of flow that considers the independent dimensions of Skill Level and Challenge placed on a person. For instance, high challenge and low skill lead to anxiety which decreases a person's productivity and reduces their ability to learn. On the other hand, low challenge and high skill level lead to boredom which usually means a person becomes disinterested in continuing with the pursuit. A state of flow where the challenge placed on a person is matched to their skill level may result in a state of flow ensues where the person is productive with seemingly little effort and negative emotion attached to the effort. The Guidance system determines a user's emotion and then adjusts the challenge placed on a person so that they are operating in a state of flow.

### Application: Monitoring Fatigue/Sleep in a Fleet of Truck Drivers

In this possible computer system implementation, truck drivers wear headsets that record their level of fatigue. The data from these headsets is sent through the cloud to the dispatch where dispatchers can monitor each driver and make sure that they are still able to drive safely.

### Application: Quality Assurance in a Factory

In this possible computer system implementation, workers in a factory wear headsets that measure their level of attention while performing for example sensitive work or potentially dangerous work. If a worker's focus drops while working on a particular piece of equipment for example, that equipment can then be marked to receive extra-attention in the quality-assurance process because of the increased likelihood of error.

### Application: Collective Brainwave Control

In other possible computer system implementations of the present invention, brainwaves may be obtained for a group of game players, and these may be processed to enable collective control such as in a cooperative multiplayer game. This type of interaction would be well suited to compassion training.

### Application: Brainwave Controlled Acoustic Attention Beam Forming

In another possible computer system implementation the MED-CASP system implementation for example may incorporate a microphone array that enables thought assisted beam-forming to allow a user to enhance a user's ability lock into directional sound in a noisy setting. The algorithm may for example associate dynamic changes in brain state with the persons desire to “tune in” to different aspects of the soundscape.

### Application: Brainwave Focus Disruptor/Encourager

User profile can keep track of patterns that affect focus. In another possible implementation of the present invention, a computer system may be provided that enables improved cognitive skills training for example using cognitive performance training, attention mastering and distraction avoidance. The computer system may be configured to affect the informatic channel between the user and their environment, to either help them focus their awareness or to break free from something that is commanding their attention in a detrimental way. This includes for instance an EEG based information filter for web browsing or other computer use. For instance it could helps make distracting advertisements or messaging disappear when you are trying to work and are having trouble concentrating. Detection for such distractions or interruptions may be performed through theta band phase locking for visual and auditory stimulus. This computer system may also detect fatigue related zoning and break the user from mind traps such as video games and web surfing. These concepts can be ultimately applied to the augmented/mediated reality applications where the audio visual signals are first intercepted by a computer system before the user received them and allows for intelligent intervention that can help a user filter out distracting information from their environment, so they can be less confused/overloaded.

Power spectrum of EEG signals from electrodes may be computed by the system of the present invention in real time. Traditional EEG bands are Delta (0 to 4 Hz), Theta (4 to 7 Hz), Alpha (8 to 13 Hz) and Beta (13 to 30 Hz). Power spectrum may be calculated as a Fast Fourier Transform of a time segment of the EEG signal. Different mathematical combinations of power spectrum measures may be calculated to derive a reward score. Examples of power spectrum measures may include a Ratio of theta (4-7 Hz) power to beta (13-30 Hz) power within a single electrode. In another example, asymmetry of an EEG band measured from two locations on the scalp of the user. In another example, the system may perform absolute or relative (ratio of one EEG band across a number of EEG bands) calculation of probabilistic distribution of an EEG band power of an individual to form a baseline. The system may reward the user for producing EEG band power above a threshold measured from the user's distribution. Normative databases of distributions of hundreds of individuals can be used to calculate threshold values. The threshold may be dynamically set such that the user receives a specified ratio of reward to no reward times. For example, a 40/60 ratio means that during a session a user will receive a reward 40% of the time and no reward (or punishment) 60% of the time. Coherence may also be used. Signals are “perfectly coherent” at a given frequency when they have both constant phase difference and constant amplitude ratio over the time considered. The coherence of two EEG signals are measured and their coherence is rewarded.

### General

It will be appreciated that any module or component exemplified herein that executes instructions may include or otherwise have access to computer readable media such as storage media, computer storage media, or data storage devices (removable and/or non-removable) such as, for example, magnetic disks, optical disks, tape, and other forms of computer readable media. Computer storage media may include volatile and non-volatile, removable and non-removable media implemented in any method or technology for storage of information, such as computer readable instructions, data structures, program modules, or other data. Examples of computer storage media include RAM, ROM, EEPROM, flash memory or other memory technology, CD-ROM, digital versatile disks (DVD), blue-ray disks, or other optical storage, magnetic cassettes, magnetic tape, magnetic disk storage or other magnetic storage devices, or any other medium which can be used to store the desired information and which can be accessed by an application, module, or both. Any such computer storage media may be part of the mobile device, tracking module, object tracking application, etc., or accessible or connectable thereto. Any application or module herein described may be implemented using computer readable/executable instructions that may be stored or otherwise held by such computer readable media.

Thus, alterations, modifications and variations can be effected to the particular embodiments by those of skill in the art without departing from the scope of this disclosure, which is defined solely by the claims appended hereto.

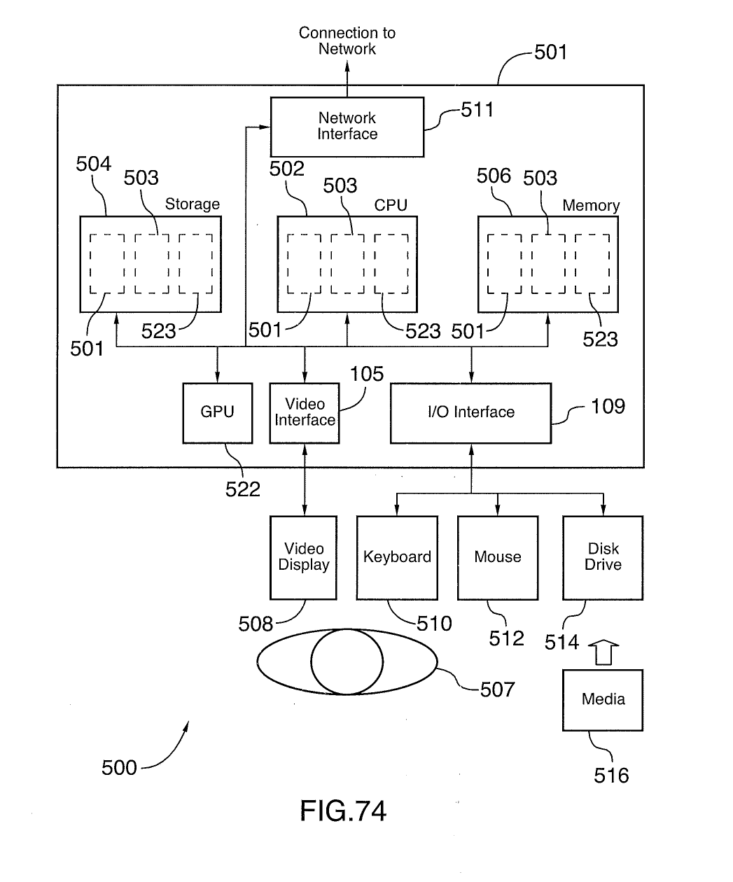

The present system and method may be practiced in various embodiments. A suitably configured computer device, and associated communications networks, devices, software and firmware may provide a platform for enabling one or more embodiments as described above. By way of example, FIG. 74 shows a generic computer device 500 that may include a central processing unit (“CPU”) 502 connected to a storage unit 504 and to a random access memory 506. The CPU 502 may process an operating system 501, application program 503, and data 523. The operating system 501, application program 503, and data 523 may be stored in storage unit 504 and loaded into memory 506, as may be required. Computer device 500 may further include a graphics processing unit (GPU) 522 which is operatively connected to CPU 502 and to memory 506 to offload intensive image processing calculations from CPU 502 and run these calculations in parallel with CPU 502. An operator 507 may interact with the computer device 500 using a video display 508 connected by a video interface 505, and various input/output devices such as a keyboard 510, mouse 512, and disk drive or solid state drive 514 connected by an I/O interface 509. In known manner, the mouse 512 may be configured to control movement of a cursor in the video display 508, and to operate various graphical user interface (GUI) controls appearing in the video display 508 with a mouse button. The disk drive or solid state drive 514 may be configured to accept computer readable media 516. The computer device 500 may form part of a network via a network interface 511, allowing the computer device 500 to communicate with other suitably configured data processing systems (not shown).

In further aspects, the disclosure provides systems, devices, methods, and computer programming products, including non-transient machine-readable instruction sets, for use in implementing such methods and enabling the functionality described previously.

Although the disclosure has been described and illustrated in exemplary forms with a certain degree of particularity, it is noted that the description and illustrations have been made by way of example only. Numerous changes in the details of construction and combination and arrangement of parts and steps may be made. Accordingly, such changes are intended to be included in the invention, the scope of which is defined by the claims.

Except to the extent explicitly stated or inherent within the processes described, including any optional steps or components thereof, no required order, sequence, or combination is intended or implied. As will be will be understood by those skilled in the relevant arts, with respect to both processes and, any systems, devices, etc., described herein, a wide range of variations is possible, and even advantageous, in various circumstances, without departing from the scope of the invention, which is to be limited only by the claims.

### REFERENCES

[1] R. Chambers, B. C. Y. Lo and N. B. Allen. The impact of intensive mindfulness training on attentional control, cognitive style, and affect. Cognitive Therapy and Research 32(3), pp. 303-322. 2008.
[2] S. G. Hofmann, A. T. Sawyer, A. A. Witt and D. Oh. The effect of mindfulness-based therapy on anxiety and depression: A meta-analytic review. J. Consult. Clin. Psychol. 78(2), pp. 169-183. 2010.
[3] A. P. Jha, E. A. Stanley, A. Kiyonaga, L. Wong and L. Gelfand. Examining the protective effects of mindfulness training on working memory capacity and affective experience. Emotion 10(1), pp. 54-64. 2010.
[4] A. Moore and P. Malinowski. Meditation, mindfulness and cognitive flexibility. Conscious. Cogn. 18(1), pp. 176-186. 2009.
[5] C. N. M. Ortner, S. J. Kilner and P. D. Zelazo. Mindfulness meditation and reduced emotional interference on a cognitive task. Motiv. Emotion 31(4), pp. 271-283. 2007.
[6] D. J. Siegel. Mindfulness training and neural integration: Differentiation of distinct streams of awareness and the cultivation of well-being. Social Cognitive and Affective Neuroscience 2(4), pp. 259-263. 2007.
[7] S. Barnes, K. W. Brown, E. Krusemark, W. K. Campbell and R. D. Rogge. The role of mindfulness in romantic relationship satisfaction and responses to relationship stress. J. Marital Fam. Ther. 33(4), pp. 482-500. 2007.
[8] A. Chiesa and A. Serretti. A systematic review of neurobiological and clinical features of mindfulness meditations. Psychol. Med. 40(8), pp. 1239-1252. 2010.
[9] Y. Tang and M. I. Posner. Attention training and attention state training. Trends Cogn. Sci. (Regul. Ed.) 13(5), pp. 222-227. 2009.
[10] Y. Tang, Q. Lu, M. Fan, Y. Yang and M. I. Posner. Mechanisms of white matter changes induced by meditation. Proc. Natl. Acad. Sci. U.S.A 109(26), pp. 10570-10574. 2012.
[11] S. Xue, Y. Tang and M. I. Posner. Short-term meditation increases network efficiency of the anterior cingulate cortex. Neuroreport 22(12), pp. 570-574. 2011.
[12] Y. Tang, Q. Lu, X. Geng, E. A. Stein, Y. Yang and M. I. Posner. Short-term meditation induces white matter changes in the anterior cingulate. Proc. Natl. Acad. Sci. U.S.A 107(35), pp. 15649-15652. 2010.
[13] M. Arns, S. de Ridder, U. Strehl, M. Breteler and A. Coenen. Efficacy of neurofeedback treatment in ADHD: The effects on inattention, impulsivity and hyperactivity: A meta-analysis. Clinical Eeg and Neuroscience 40(3), pp. 180-189. 2009.
[14] S. Niv. Clinical efficacy and potential mechanisms of neurofeedback. Personality and Individual Differences 54(6), pp. 676-686. 2013.
[15] S. L. Shapiro, L. E. Carlson, J. A. Astin, and B. Freedman. Mechanisms of Mindfulness. Journal of Clinical Psychology DOI 10.1002/jclp.20237. 2005.

Any and all features of novelty disclosed or suggested herein, including without limitation the following:

What is claimed is:

1. A system comprising:
at least one computing device comprising at least one processor and at least one non-transitory computer readable medium storing computer processing instructions;
at least one bio-signal sensor in communication with the at least one computing device;
wherein, upon execution of the computer processing instructions by the at least one processor, the at least one computing device is configured to:
execute at least one brain state guidance routine comprising at least one brain state guidance objective;
present at least one brain state guidance indication at the at least one computing device for presentation to at least one user, in accordance with the executed at least one brain state guidance routine;
receive bio-signal data of the at least one user from the at least one bio-signal sensor, at least one of the at least one bio-signal sensor comprising at least one brainwave sensor, and the received bio-signal data comprising at least brainwave data of the at least one user;
measure performance of the at least one user relative to at least one brain state guidance objective corresponding to the at least one brain state guidance routine at least partly by analyzing the received bio-signal data; and
update the presented at least one brain state guidance indication based at least partly on the measured performance.

2. The system of claim 1 wherein the at least one brain state guidance objective comprises at least one brain state stability threshold, and the analyzing the received bio-signal data comprises determining a stability of at least the brainwave data in comparison to the at least one brain state stability threshold.

3. The system of claim 2 wherein the brain state guidance exercise comprises a meditation exercise, and the updating comprises presenting an indication of the stability of the brainwave data of the at least one user.

4. The system of claim 2 wherein the at least one computing device is configured to calibrate the at least one brain state stability threshold at least partly by:
analyzing fixed time segments of the brainwave data in real-time;
calculating an alpha power value for each time segment;
calculating a statistical distribution of the alpha power values over a predetermined calibration time segment, the predetermined calibration time segment comprising a longer duration than each of the fixed time segments; and
determining a statistical distribution of alpha variability based at least partly on instantaneous alpha variability of the statistical distribution of the alpha power values.

5. The system of claim 4 wherein the received bio-signal data is time-coded, and the stability determining comprises:
determining alpha power and alpha variability of the brainwave data for respective time codes;
comparing the determined alpha power to the statistical distribution of the alpha power values;
comparing the determined alpha variability to the statistical distribution of the alpha variability; and
based at least on the alpha power comparison and the alpha variability comparison, estimating a busy-mind score on a continuum from quiet-mind to busy-mind,
wherein the at least one brain state guidance indication updating is based at least partly on the estimated busy-mind score.

6. The system of claim 5 wherein the continuum is quantized into a predetermined number of quantization segments, the at least one brain state guidance indication comprises a respective brain state guidance indication state corresponding to each quantization segment, the at least one brain state guidance indication updating based at least partly on the brain state guidance indication state corresponding to a determined quantization segment corresponding to the estimated busy-mind score.

7. The system of claim 5 wherein the at least one brain state guidance indication comprises a representation of blowing wind, and the at least one brain state guidance indication updating comprises updating the intensity of the representation of the blowing wind based at least partly on the estimated busy-mind score.

8. The system of claim 7 wherein the intensity of the representation of the blowing wind is increased based at least partly on the busy-mind score estimated to be in a busy-mind state on the continuum.

9. The system of claim 5 wherein the at least one brain state guidance indication updating comprises presenting a representation of a bird song based at least partly on the estimated busy-mind score remaining in a quiet-mind state on the continuum for a predetermined time period.

10. The system of claim 5 wherein the stability determining is periodically re-determined at a time interval.

11. The system of claim 10 wherein the time interval is 1/10 of a second.

12. The system of claim 5 wherein the at least one computing device is configured to monitor for a statistically significant change in the determined alpha power and alpha variability of the brainwave data, and in accordance with determining the statistically significant change has occurred, repeat the at least one brain state stability threshold calibrating.

13. The system of claim 5 wherein the at least one brain state guidance objective comprises the estimated busy-mind score indicative of a quiet-mind on the continuum.

14. The system of claim 5 wherein the at least one computing device is configured to present a representation of awarding a milestone reward to the user based at least partly on the user achieving the at least one brain state guidance objective.

15. The system of claim 5 wherein the at least one brain state guidance indication directs the user to enter a quiet-mind state.

16. The system of claim 5 wherein the at least one brain state guidance indication directs the user to enter a quiet-mind state, then enter a busy-mind state, then enter a quiet-mind state again.

17. The system of claim 1 wherein the at least one computing device comprises a display, the at least one brain state guidance indication updating comprising displaying a visual prompt on the display for directing the user.

18. The system of claim 1 wherein the at least one computing device comprises an audio output device, the at least one brain state guidance indication updating comprising playing an audio prompt on the audio output device for directing the user.

19. The system of claim 1 wherein the at least one computing device is configured to generate a feedback report for at least one session of execution of the brain state guidance routine providing an indication of the measured performance.

20. The system of claim 1 wherein execution of the brain state guidance routine is time-coded, the received bio-signal data is time-coded, and the at least one computing device configured to update a bio-signal interaction profile associated with the at least one user with the time-coded bio-signal data and the measured performance.

21. The system of claim 20 wherein the performance measuring is further based on data stored in the bio-signal interaction profile of the at least one user.

22. The system of claim 20 wherein the at least one brain state guidance objective is based at least partly on data stored in the bio-signal interaction profile of the at least one user.

23. The system of claim 1 wherein one of the at least one bio-signal sensor comprises at least one of a heart-rate monitor, blood pressure monitor, blood glucose monitor, and accelerometer.

24. The system of claim 1 wherein the at least one computing device is configured to transmit the measured performance to at least one remote computing device over a communications network.

25. The system of claim 24 wherein the updating the presented at least one brain state guidance indication is based at least partly on guidance information received from the remote computing device.

26. The system of claim 1 wherein the at least one brain state guidance routine is controlled by a remote computing device in communication with the at least one computing device over a communications network.

27. The system of claim 1 wherein the at least one computing device is configured to notify a remote computing device upon execution of the at least one brain state guidance routine.

28. The system of claim 1 wherein the bio-signal data analyzing is based at least partly on receiving brain state metadata information from the user, the at least one computing device configured to tag the bio-signal data with the received brain state metadata information, and store the tagged bio-signal data in a bio-signal interaction profile associated with the at least one user.

29. The system of claim 28 wherein the at least one brain state guidance objective is based at least partly on the received brain state metadata information.

30. The system of claim 1 wherein the at least one computing device is configured to receive brain state guidance proficiency information indicating the proficiency of the respective user in achieving brain state guidance objectives, and to select the brain state guidance routine for execution based at least partly on the received proficiency information.

31. The system of claim 30 wherein the at least one computing device is configured to generate the brain state guidance proficiency information based at least partly on the measured performance.

32. A method performed by at least one computing device in communication with at least one bio-signal sensor, the at least one computing device comprising at least one processor and at least one non-transitory computer readable medium storing computer processing instructions, that when executed by the at least one computer processor, cause the at least one computing device to perform the method, the method comprising:
executing at least one brain state guidance routine comprising at least one brain state guidance objective;
presenting at least one brain state guidance indication at the at least one computing device for presentation to at least one user, in accordance with the executed at least one brain state guidance routine;
receiving bio-signal data of the at least one user from the at least one bio-signal sensor, at least one of the at least one bio-signal sensor comprising at least one brainwave sensor, and the received bio-signal data comprising at least brainwave data of the at least one user;
measuring performance of the at least one user relative to at least one brain state guidance objective corresponding to the at least one brain state guidance routine at least partly by analyzing the received bio-signal data; and
updating the presented at least one brain state guidance indication based at least partly on the measured performance.
